(19) United States
(12) Patent Application Publication
(10) Pub. No.: US 2025/0230248 A1
(43) Pub. Date: Jul. 17, 2025

(54) ANTI-EGFR ANTIBODIES, ANTI-CMET ANTIBODIES, ANTI-VEGF ANTIBODIES, MULTISPECIFIC ANTIBODIES, AND USES THEREOF

(71) Applicant: TAVOTEK BIOTHERAPEUTICS (HONG KONG) LIMITED, Hong Kong (CN)

(72) Inventors: Pu PU, Hong Kong (CN); Songling ZHANG, Hong Kong (CN); Ying JIN, Hong Kong (CN); Maria P. MACWILLIAMS, Lower Gwynedd, PA (US); Man-Cheong FUNG, Lower Gwynedd, PA (US); Mark L. CHIU, Lower Gwynedd, PA (US)

(73) Assignee: TAVOTEK BIOTHERAPEUTICS (HONG KONG) LIMITED, Hong Kong (CN)

(21) Appl. No.: 18/701,582

(22) PCT Filed: Oct. 17, 2022
(86) PCT No.: PCT/US2022/078192

§ 371 Date: Apr. 15, 2024

Related U.S. Application Data
(60) Provisional application No. 63/256,705, filed on Oct. 18, 2021.

(51) Int. Cl. C07K 16/28; A61K 39/00; A61P 35/00; C07K 16/22; C07K 16/40

(52) U.S. Cl. CPC C07K 16/2863; A61P 35/00; C07K 16/22; C07K 16/40; C12Y 207/10001; A61K 2039/505; C07K 2317/24; C07K 2317/31; C07K 2317/35; C07K 2317/732; C07K 2317/734; C07K 2317/76; C07K 2317/92; C07K 2317/94

(57) ABSTRACT

The disclosure provides antibodies and fragments targeting EGFR, VEGF, PD-L1, or cMET. The disclosure also provides multispecific antibodies that comprise a first variable domain that can bind the epidermal growth factor receptor (EGFR), a second variable domain that can bind cMET, and a third variable domain that can bind PD-L1 or VEGF. The multispecific antibodies are effective in treating cancers and/or other diseases, disorders, and conditions where pathogenesis is mediated by EGFR, VEGF or PD-L1, and cMET.

### CROSS-REFERENCE TO RELATED APPLICATION

This application claims priority to U.S. Provisional Application No. 63/256,705, filed on Oct. 18, 2021, the contents of which are hereby incorporated by reference.

### SEQUENCE LISTING

This application contains a Sequence Listing electronically submitted as an XML file entitled “15271.0011-00304_sql_20221014.xml” having a size of 140,305 bytes and created on Oct. 14, 2022. The information contained in the Sequence Listing is incorporated by reference herein.

### FIELD OF THE DISCLOSURE

The present disclosure relates to antibodies that target the epidermal growth factor receptor (EGFR), cMET, and the PD-L1/VEGF axis, and uses of the antibodies, to treat or prevent cancers and other diseases, disorders, and conditions where pathogenesis is mediated by EGFR, PD-L1/VEGF, and/or cMET.

### BACKGROUND OF THE DISCLOSURE

The epidermal growth factor (EGF) receptor (EGFR) is a cell-surface receptor and is also known as the ErbB-1 receptor, ERBB, ERBB1, HER1, PIG61, and mENA. EGFR is a member of the ErbB family of receptors, a subfamily of four closely related receptor tyrosine kinases: ErbB-1 (EGFR), ErbB-2 (HER2/c-neu; Her 2), ErbB-3 (Her 3) and ErbB-4 (Her 4). EGFR is a member of the type 1 tyrosine kinase family of growth factor receptors, which plays critical roles in cellular growth, differentiation, and survival.

EGFR can be activated by specific ligands, including epidermal growth factor, amphiregulin, heparin-binding ETF, betacellulin, and transforming growth factor alpha (TGF a). Upon activation by its growth factor ligands, the receptor may undergo a transition from an inactive, mostly monomeric form to an active homodimer. In addition to forming homodimers after ligand binding, EGFR may pair with another member of the ErbB receptor family, such as ErbB-2, to form activated heterodimers in the absence of ligand-binding.

Mutations involving EGFR have been identified in several types of cancer. Small-molecule tyrosine kinase inhibitors (TKIs) of the first generation of TKIs, e.g., gefitinib and erlotinib, block autophosphorylation of EGFR in the intracellular tyrosine kinase region, thereby inhibiting downstream signaling events. Lung cancer cell lines such as H1975 (L858R/T790M) and H820 (del (E746, A750), T790M) due to the T790M mutation are resistant to 1^{st} generation TKI molecules. The 2^{nd} generation of TKIs, e.g., Afatinib, Dacomitinib, and Neratinib, had promising activity against EGFR T790M in animal models but displayed limited clinical efficacy due to dose-limiting toxicity caused by simultaneous inhibition of wild-type EGFR. The H1975-HGF xenograft model is resistant to Erlotinib and Afatinib shown in FIG. 13 of Janssen's patent US2018/0258173 A1. The 3^{rd} generation TKI, Osimertinib, has been approved for non-small cell lung cancer (NSCLC) patients that have acquired the EGFR T790M resistance mutation. However, patients treated with Osimertinib eventually acquire drug resistance. An emerging frequent mechanism is mutation of EGFR (C797S). Multiple studies have highlighted activation of cMet, by increased expression of cMET due to MET gene amplification or by increased expression of HGF, as an important resistance mechanism to both 1^{st}- and 3^{rd} generation TKIs.

Both cetuximab and panitumumab function by targeting the extracellular portion of EGFR and blocking ligand binding, thereby inhibit downstream events leading to the inhibition of cell proliferation. However, patients whose tumor contains other mutations usually do not benefit from cetuximab or panitumumab therapy. KRAS gain-of-function mutations alter signaling properties in the tumor cells by continuously sending a growth signal even if EGFR has been blocked. Side effects of current EGFR-targeted therapies targeting EGFR overexpressing cancer cells suffer from toxicities due to basal expression of EGFR in other normal tissues outside of the tumor. Activating mutations in the EGFR gene, mainly L858R mutation, exon 19 deletions, and exon 20 mutations result in ligand-independent activation of the EGFR kinase activity. Aberrant activation of both EGFR and mesenchymal-epithelial transition factor (MET) signaling pathways has been implicated in driving tumor cell growth and proliferation in lung cancer (Bean, Brennan et al. 2007, Engelman, Zejnullahu et al. 2007).

MET is the human receptor for human hepatocyte growth factor (HGF; also known as scatter factor), a member of the tyrosine kinase superfamily. The cMET ligands are potent mitogens/morphogens which include HGF, and its splicing isoforms (NK1, NK2). Expression of HGF is also associated with the activation of the HGF/cMET signaling pathway and is also one of the escape mechanisms of tumors under selection by EGFR-targeted therapy. Binding of ligands to cMET leads to receptor multimerization, phosphorylation of multiple tyrosine residues in the intracellular region, and catalytic activation-of downstream signaling. The HGF/cMET signaling pathway plays important roles in normal body development and wound healing. However, abnormal cMET activation in cancer results in tumor progression, angiogenesis, invasive growth, and metastasis of cancers. Dysregulation and/or hyperactivation of HGF or cMET in human cancers via overexpression, amplification, or mutation are linked to poor prognosis. cMET can be activated in an HGF associated and HGF independent manner. Overexpression of cMET, MET gene amplification or mutation has been reported in various cancers such as colorectal, lung, gastric, and kidney cancer and may drive ligand-independent receptor activation (Birchmeier, Birchmeier et al. 2003). Abundance of cMET also may trigger homodimerization and heterodimerization and subsequently activate the intracellular signaling in the absence of ligand.

MET and EGFR are also co-expressed in many human tumors. Blocking one receptor tends to up-regulate the other, frequently and often quickly leads to resistance to single anti-tumor agent treatment (Engelman, Zejnullahu et al. 2007). Conversely, cMET-amplified lung cancer cells exposed to cMET-inhibiting agents for a prolonged period develop resistance via the EGFR pathway (McDermott, Pusapati et al. 2010). The cMET/HGF signaling in resistance to EGFR-targeted therapies has fostered the development of molecules to treat the resistance. Unfortunately, antibody based approaches include anti-HGF antibodies, anti cMET antibodies have not been clinically effective (Lee, Sung et al. 2015). In addition, several in vivo studies showed that some cMET small molecule inhibitors have potential side effects, such as heart rate acceleration, cardiac muscle denaturation, renal toxicity, and body weight reduction (Cui, Shen et al. 2013).

To address the challenge of shutting down both EGFR and cMET pathways, various multispecific EGFR×cMET antibodies have been developed. There are varying architectures that include: Samsung ME22S having a 2+2 single chain (sc) Fv-mAb fusion format (US2015030599); Merck with SEED 1+1 multispecific antibody format (WO2017/076492); Genentech with 2:1 scFv of cMET fused on a cetuximab; Roche Glycart with 1+1 scFab cMET fused to cetuximab format (UWO2017/076492); Lilly LY3164530 with 2+2 scFv-Fab-Fc/scFv-Fab format (US20130156772); Epimab with FIT Ig 2+2 format; Merus MCLA-129 with common light chain 1+1 format (US2020024892); and the Janssen/Genmab 1+1 DuoBody format (US20180258173). Each molecule has differing ranges of efficacies but still suffer from the application limited to certain patient subsets due to the induction of resistance as well as the occurrence of dose limiting adverse effects.

Programmed death ligand-1 (PD-L1) is expressed in 19.6%-65.3% of NSCLC (Pawelczyk, Piotrowska et al. 2019). In addition, the presence of EGFR mutations are linked to PD-L1 expression. EGFR activation by EGF stimulation, exon-19 deletions, and L858R mutation could also induce PD-L1 expression. Such EGFR activation can induce the apoptosis of T cells through the PD-L1/VEGF axis in tumor cells and peripheral blood mononuclear cells coculture systems. Thus, inhibiting EGFR by EGFR-TKIs could free the inhibition of T cells and enhance the production of interferon-y (Chen, Fang et al. 2015, Fan, Liu et al. 2015). A targeted cMET and programmed death-1 (PD-1) humanized multispecific monoclonal antibody was developed to inhibit tumor progression, migration, metastasis, and angiogenesis by blocking cMET, and can also rescue systemic T cell function by blocking PD-1 in cancer cells overexpressing cMET and PD-L1. Moreover, such a BsAb could bridge T cells and tumor cells, allowing the T cells to target the tumor cells directly (Sun, Wu et al. 2017). In other studies, a multispecific cMET/PD-L1 CAR-T is more effective than monovalent cMET CAR-T for the treatment of hepatocellular carcinoma. In vivo experiments have demonstrated that the cMET/PD-L1 CAR-T cells significantly inhibited tumor growth and improved survival persistence (Jiang, Li et al. 2021).

Currently, immunotherapies involving PD-1 and its respective ligand PD-L1 are promising. PD-L1 (also known as B7-H1 or CD274) is a cognate ligand for PD-1 which is overexpressed in a variety of tumors. The binding of PD-1 and PD-L1 can inhibit NK cell and T cell activation, proliferation, and survival which eventually leads to the immune evasion of tumor cells. Recent studies have demonstrated that blocking the PD-1/PD-L1 pathway can enhance the endogenous antitumor immunity by restoring the action of T lymphocytes. Thus, manipulating PD-1/PD-L1 axis might be a promising treatment option for NSCLC. Anti-PD-1/PD-L1 antibodies could be an optional therapy for EGFR-TKI resistant patients, especially for EGFR-TKIs resistant NSCLC patients with EGFR mutation.

To obtain a better strategy to contain EGFR+ cancers, alternative therapeutics have been developed. Interactions between cancer cells and their microenvironment are critical for the development and progression of solid tumors (Holmgren, O'Reilly et al. 1995). Tumor growth and metastasis are critically dependent on the development and/or remodeling of the microvasculature (Folkman 1995). Inflammatory breast cancer is characterized pathologically by high vascularity and increased micro vessel density because of high expression of angiogenic factors such as VEGF which is a key mediator of angiogenesis and is involved in endothelial and tumor cell growth and motility and blood vessel permeability (Kaumaya and Foy 2012). The transition between dormancy and active growth in tumorigenesis appears to be triggered by an “angiogenic switch” (Holmgren, O'Reilly et al. 1995). This angiogenic switch has recently been documented in several forms of cancer. VEGF constitutes one of the most proangiogenic factors known today (Troiani, Martinelli et al. 2012). In many different types of cancer, the gene expression, and levels of secretion of VEGF are elevated (Fukumura, Xavier et al. 1998). VEGF is a key promoter of metastasis as well as serves as an angiogenesis factor via VEGF receptor (VEGFR)-1 and/or VEGFR-2 signaling. Endothelial cells respond via VEGFR-2 activation, while infiltrating cells, such as macrophages, are activated via VEGFR-1 signaling, which is also involved in the recruitment of endothelial progenitor cells in neovascularization (Kaumaya and Foy 2012).

Angiogenesis is implicated in the pathogenesis of a variety of disorders which include solid tumors, intraocular neovascular syndromes such as proliferative retinopathies or age-related macular degeneration (AMD), rheumatoid arthritis, and psoriasis (Klagsbrun and D'Amore 1991, Folkman and Shing 1992). In the case of solid tumors, the neovascularization allows the tumor cells to acquire a growth advantage and proliferative autonomy compared to the normal cells. Accordingly, a correlation has been observed between density of micro vessels in tumor sections and patient survival in breast cancer as well as in several other tumors (Weidner, Semple et al. 1991, Horak, Leek et al. 1992, Macchiarini, Fontanini et al. 1992).

To address one or more challenges with human tumors with anti-EGFR, cMET, PD-L1/VEGF, and HGF immunotherapies, the present disclosure provides novel anti-EGFR, anti-cMET, and anti-VEGF antibodies. The present disclosure also provides novel multispecific antibodies such as a multispecific antibody that comprises a first variable domain that can bind EGFR (e.g., an extracellular domain of EGFR), a second variable domain that can bind to cMET (e.g., an extracellular domain of cMET), and a third variable domain that can block PD-L1 or VEGF.

A multifunctional antibody of the present disclosure that binds cMET, PD-L1/VEGF, and EGFR with high affinity can provide one or more benefits of the following. For example, a multifunctional antibody of the present disclosure can effectively neutralize cMET activation by HGF and EGFR activation by EGF family and HGF family ligands, and/or provides superior activity in internalizing and/or degrading cMET and EGFR (both wild-type and mutants) relative to combinations of single agents. Such a multifunctional antibody is needed as an effective pharmacological intervention for certain cancers. Such a multifunctional antibody can prevent the potential of heterodimer cluster formation between EGFR, cMET, and HER family members. Such a multifunctional antibody can block the PD-1 and PD-L1 engagement to enhance the activity of immune cells in the vicinity. Particularly, a multifunctional antibody of the present disclosure can (a) more effectively treat cancers characterized by having one or more KRAS and Exon20 mutations; (b) demonstrate superior activity in preventing or delaying the development of resistance to other cMET and/or EGFR inhibitors including, but not limited to, erlotinib, gefitinib, lapatinib and vemurafenib, as compared to relevant combinations of single agents; (c) elicit minimal or no measurable agonist EGFR and cMET activity; (d) block the PD-L1 and PD-1 engagement; or block the VEGF activity of angiogenesis; and/or (f) demonstrate in vivo stability, physical and chemical stability including, but not limited to, thermal stability, solubility, low self-association, and pharmacokinetic characteristics which are acceptable for development and/or use in the treatment of cancer.

### SUMMARY OF THE DISCLOSURE

The present disclosure provides antibodies or antigen-binding fragments thereof directed against EGFR, PD-L1/VEGF, and cMET, nucleic acids encoding such antibodies and fragments, methods for preparing the antibodies and fragments, and methods for the treatment of diseases, such as EGFR, PD-L1/VEGF, and cMET mediated diseases or disorders, e.g., human cancers, including lung, head and neck, kidney, liver, gastric, colorectal, triple negative breast, pancreatic, and neuroendocrine cancers.

In one aspect, the present disclosure provides an anti-EGFR antibody or an antigen-binding fragment thereof. In some embodiments, the present disclosure provides an anti-EGFR antibody or an antigen-binding fragment thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NOs: 95, 96, and 97;
SEQ ID NOs: 95, 96, and 98;
SEQ ID NOs: 95, 96, and 105;
SEQ ID NOs: 102, 100, and 101; and
SEQ ID NOs: 102, 103, and 104; respectively.

In some embodiments, the disclosure provides an anti-EGFR antibody or antigen binding fragment thereof comprising at least one antibody single domain selected from SEQ ID NOs: 5-12 or antigen binding fragment thereof. In some embodiments, the anti-EGFR antibody or antigen binding fragment thereof comprises tandem antibody single domain heavy chains, optionally linked via a linker. In some embodiments, the anti-EGFR antibody or antigen binding fragment thereof comprises tandem antibody single domain heavy chains selected from SEQ ID NOs: 13-18 or antigen binding fragment thereof, wherein two EGFR-binding VHO sequences are linked via a linker.

In some embodiments, the disclosure provides an anti-EGFR antibody or antigen binding fragment thereof comprising at least one antibody single domain having at least 85% identity to any one of SEQ ID NOs: 5-12 or antigen binding fragment thereof. In some embodiments, the anti-EGFR antibody or antigen binding fragment thereof comprises tandem antibody single domain heavy chains having at least 85% identity to any one of SEQ ID NOs: 13-18 or antigen binding fragment thereof.

In some embodiments, the disclosure provides an anti-EGFR antibody or antigen binding fragment thereof that binds one or more epitopes on EGFR (e.g., human EGFR) recognized by an anti-EGFR antibody or antigen binding fragment thereof comprising at least one antibody single domain selected from SEQ ID NOs: 5-12 or comprising tandem antibody single domain heavy chains selected from SEQ ID NOs: 13-18.

In some embodiments, the disclosure provides an anti-EGFR antibody or antigen binding fragment thereof comprising human antibody heavy chain SEQ ID NO: 1 and human antibody light chain SEQ ID NO: 2; or human antibody heavy chain SEQ ID NO: 3 and human antibody light chain SEQ ID NO: 4.

In another aspect, the present disclosure provides an anti-cMET antibody or an antigen-binding fragment thereof. In some embodiments, the present disclosure provides an anti-cMET antibody or an antigen-binding fragment thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NOs: 106, 107, and 133;
SEQ ID NOs: 111, 112, and 113;
SEQ ID NOs: 111, 114, and 113;
SEQ ID NOs: 99, 118, and 119;
SEQ ID NOs: 99, 120, and 119;
SEQ ID NOs: 99, 121, and 119; and
SEQ ID NOs: 99, 122, and 119; respectively.

In some embodiments, the present disclosure provides an anti-cMET antibody or an antigen-binding fragment thereof, comprising a light chain variable region comprising three CDRs, designated as LCDR1, LCDR2, and LCDR3, wherein the LCDR1, LCDR2, and LCDR3 are selected from:
SEQ ID NOs: 108, 109, and 110;
SEQ ID NOs: 115, 116, and 117; and
SEQ ID NOs: 123, 124, and 125; respectively.

In some embodiments, the present disclosure provides an anti-cMET antibody or an antigen-binding fragment thereof comprising an antibody heavy chain sequence selected from SEQ ID NOs: 24, 28-29, and 34-37, and an antibody light chain sequence selected from SEQ ID NOs: 26, 31-32, and 39-40. In some embodiments, the present disclosure provides an anti-cMET antibody or an antigen-binding fragment thereof comprising an antibody heavy chain sequence selected from SEQ ID NOs: 23, 27, and 33, and an antibody light chain sequence selected from SEQ ID NOs: 25, 30, and 38. In some embodiments, the disclosure provides an anti-cMET antibody or an antigen-binding fragment thereof comprising at least one cMET binding VHO (variable heavy chain only) sequence selected from SEQ ID NOs: 41-44.

In some embodiments, the present disclosure provides an anti-cMET antibody or an antigen-binding fragment thereof comprising an antibody heavy chain sequence having at least 85% identity to any one of SEQ ID NOs: 23, 24, 27-29, and 33-37, and an antibody light chain sequence having at least 85% identity to any one of SEQ ID NOs: 25, 26, 30-32, and 38-40. In some embodiments, the disclosure provides an anti-cMET antibody or an antigen-binding fragment thereof comprising at least one cMET binding VHO sequence having at least 85% identity to any one of SEQ ID NOs: 41-44.

In some embodiments, the disclosure provides an anti-cMET antibody or antigen binding fragment thereof that binds one or more epitopes on cMET recognized by an anti-cMET antibody or antigen binding fragment thereof comprising an antibody heavy chain sequence selected from SEQ ID NOs: 23, 24, 27-29, and 33-37, and an antibody light chain sequence selected from SEQ ID NOs: 25, 26, 30-32, and 38-40. In some embodiments, the disclosure provides an anti-cMET antibody or antigen binding fragment thereof that binds one or more epitopes on cMET recognized by an anti-cMET antibody or antigen binding fragment thereof comprising at least one cMET binding VHO sequence selected from SEQ ID NOs: 41-44.

In some embodiments, the present disclosure provides an anti-cMET antibody or an antigen-binding fragment thereof comprising a human antibody heavy chain and a human antibody light chain selected from human antibody heavy chain SEQ ID NO: 23 and human antibody light chain SEQ ID NO: 25; human antibody heavy chain SEQ ID NO: 23 and human antibody light chain SEQ ID NO: 26; human antibody heavy chain SEQ ID NO: 24 and human antibody light chain SEQ ID NO: 25; human antibody heavy chain SEQ ID NO: 24 and human antibody light chain SEQ ID NO: 26; human antibody heavy chain SEQ ID NO: 27 and human antibody light chain SEQ ID NO: 30; human antibody heavy chain SEQ ID NO: 27 and human antibody light chain SEQ ID NO: 31; human antibody heavy chain SEQ ID NO: 27 and human antibody light chain SEQ ID NO: 32; human antibody heavy chain SEQ ID NO: 28 and human antibody light chain SEQ ID NO: 30; human antibody heavy chain SEQ ID NO: 28 and human antibody light chain SEQ ID NO: 31; human antibody heavy chain SEQ ID NO: 28 and human antibody light chain SEQ ID NO: 32; human antibody heavy chain SEQ ID NO: 29 and human antibody light chain SEQ ID NO: 30; human antibody heavy chain SEQ ID NO: 29 and human antibody light chain SEQ ID NO: 31; human antibody heavy chain SEQ ID NO: 29 and human antibody light chain SEQ ID NO: 32; human antibody heavy chain SEQ ID NO: 33 and human antibody light chain SEQ ID NO: 38; human antibody heavy chain SEQ ID NO: 33 and human antibody light chain SEQ ID NO: 39; human antibody heavy chain SEQ ID NO: 33 and human antibody light chain SEQ ID NO: 40; human antibody heavy chain SEQ ID NO: 34 and human antibody light chain SEQ ID NO: 38; human antibody heavy chain SEQ ID NO: 34 and human antibody light chain SEQ ID NO: 39; human antibody heavy chain SEQ ID NO: 34 and human antibody light chain SEQ ID NO: 40; human antibody heavy chain SEQ ID NO: 35 and human antibody light chain SEQ ID NO: 38; human antibody heavy chain SEQ ID NO: 35 and human antibody light chain SEQ ID NO: 39; human antibody heavy chain SEQ ID NO: 35 and human antibody light chain SEQ ID NO: 40; human antibody heavy chain SEQ ID NO: 36 and human antibody light chain SEQ ID NO: 38; human antibody heavy chain SEQ ID NO: 36 and human antibody light chain SEQ ID NO: 39; human antibody heavy chain SEQ ID NO: 36 and human antibody light chain SEQ ID NO: 40; human antibody heavy chain SEQ ID NO: 37 and human antibody light chain SEQ ID NO: 38; human antibody heavy chain SEQ ID NO: 37 and human antibody light chain SEQ ID NO: 39; human antibody heavy chain SEQ ID NO: 37 and human antibody light chain SEQ ID NO: 40.

In another aspect, the present disclosure provides an anti-PDL-1 antibody or an antigen-binding fragment thereof comprising an amino acid sequence selected from SEQ ID NOs: 71-72.

In some embodiments, the present disclosure provides an anti-PD-L1 antibody or an antigen-binding fragment thereof comprising an amino acid sequence having at least 85% identity to any one of SEQ ID NOs: 71-72.

In some embodiments, the disclosure provides an anti-PD-L1 antibody or antigen binding fragment thereof that binds one or more epitopes on PD-L1 recognized by an anti-PD-L1 antibody or antigen binding fragment thereof comprising an amino acid sequence selected from SEQ ID NOs: 71-72.

In another aspect, the present disclosure provides an anti-VEGF antibody or an antigen-binding fragment thereof. In some embodiments, the present disclosure provides an anti-VEGF antibody or an antigen-binding fragment thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NOs: 129, 130, and 131; and
SEQ ID NOs: 129, 132, and 131; respectively.

In some embodiments, the present disclosure provides an anti-VEGF antibody or an antigen-binding fragment thereof, comprising a light chain variable region comprising three CDRs, designated as LCDR1, LCDR2, and LCDR3, wherein the LCDR1, LCDR2, and LCDR3 are: SEQ ID NOs: 126, 127, and 128, respectively.

In some embodiments, the present disclosure provides an anti-VEGF antibody or an antigen-binding fragment thereof comprising an amino acid sequence selected from SEQ ID NOs: 73-76.

In some embodiments, the present disclosure provides an anti-VEGF antibody or an antigen-binding fragment thereof comprising an amino acid sequence having at least 85% identity to any one of SEQ ID NOs: 73-76.

In some embodiments, the disclosure provides an anti-VEGF antibody or antigen binding fragment thereof that binds one or more epitopes on VEGF recognized by an anti-VEGF antibody or antigen binding fragment thereof comprising an amino acid sequence selected from SEQ ID NOs: 73-76.

In another aspect, the present disclosure provides multispecific antibodies that bind EGFR and cMET, as well as VEGF or PD-L1, and that exhibit one or more desirable functional properties. Such properties include, for example, high affinity specific binding to human EGFR and cMET, capability of blocking the EGFR ligands such as EGF from binding to EGFR, capability of blocking cMET ligands such as HGF to cMET, capability of binding to PD-L1 or VEGF, and capability of blocking PD-1 from binding to PD-L1.

In some embodiments, a bivalent anti-cMET antibody binding to cMET can result in tumor cell proliferation. Thus, a multispecific antibody disclosed herein preferably has monovalent cMET-binding (i.e., one Fab arm binding to an epitope of cMET).

Some embodiments provide for a multispecific antibody with cMET binding valency of 1.

Some embodiments provide for a multispecific antibody with EGFR binding valency of 1 or 2.

Some embodiments provide for a multispecific antibody with PD-L1 or PD-1 binding valency of 1 or 2.

In some embodiments, the disclosure provides a trispecific antibody that comprises an EGFR arm that can bind EGFR (e.g., human EGFR), a cMET arm that can bind cMET (e.g., human cMET), a third variable domain that can bind PD-L1 (e.g., human PD-L1) or VEGF (e.g., human VEGF). The antibody may be a full-length antibody in an IgG1 format having an anti-EGFR, anti-cMET, and anti-PD-L1 or anti-VEGF stoichiometry of 2:1:1 or 2:1:2.

In some embodiments, the disclosure provides a trispecific antibody that comprises an EGFR arm comprising a first variable domain that can bind EGFR (e.g., an extracellular domain of EGFR), a cMET arm comprising a second variable domain that can bind cMET (e.g., an extracellular domain of cMET), and a third variable domain that can bind PD-L1 or VEGF. The antibody may be a full-length antibody in an IgG1 format having an anti-EGFR, anti-cMET, and anti-PD-L1 or anti-VEGF stoichiometry of 2:1:1 or 2:1:2.

In some embodiments, the present disclosure provides a multispecific antibody that comprises a binding arm that can target EGFR which can include, but not limited to, a human IgG heavy chain fusion comprising amino acid sequences from the N- to the C-terminus, signal sequence A-shield A-linker A-protease sequence A-linker B-VHO-A targeting EGFR-linker C-VHO-B targeting EGFR-linker D-Fc. In some embodiments, the present disclosure provides a multispecific antibody comprising a binding arm that can target both EGFR and PD-L1/VEGF, comprising a human IgG heavy chain fusion comprising signal sequence A-shield A-linker A-protease sequence A-linker B-VHO-A targeting EGFR-linker C-VHO-B targeting EGFR-linker D-Fc-anti-PD-L1/VEGF. The IgG can have human IgG1, IgG2, IgG3, and/or IgG4 Fc frameworks.

In some embodiments, the present disclosure provides a multispecific antibody comprising a binding arm that can target cMET which can include, but not limited to, a human IgG heavy chain fusion comprising amino acid sequences from the N- to the C-terminus, signal sequence C-shield C-linker E-protease sequence B-linker F-IgG1 heavy chain targeting cMET; and a human IgG light chain fusion comprising amino acid sequences from the N- to the C-terminus, the signal sequence D-shield D-linker G-protease sequence C-linker H-IgG light chain targeting cMET. In some embodiments, the present disclosure provides a multispecific antibody comprising a binding arm that can target both cMET and PD-L1/VEGF comprising a human IgG heavy chain fusion comprising amino acid sequences from the N- to the C-terminus, the signal sequence C-shield C-linker E-protease sequence B-linker F-IgG1 heavy chain targeting cMET-anti-PD-L1/VEGF; and a human IgG light chain fusion comprising amino acid sequences from the N- to the C-terminus, signal sequence D-shield D-linker G-protease sequence C-linker H-IgG1 light chain targeting cMET-anti-PD-L1/VEGF. In some embodiments, the present disclosure provides a multispecific antibody comprising a binding arm that can target both cMET and PD-L1/VEGF, comprising a human IgG heavy chain fusion comprising signal sequence A-shield A-linker A-protease sequence A-linker B-VHO-A targeting cMET-linker C-VHO-B targeting cMET-linker D-Fc-anti-PD-L1/VEGF. The IgG can have human IgG1, IgG2, IgG3, and/or IgG4 Fc frameworks.

The shields (shield A, B, C, D) can be the same or different. The linkers (linker A, B, C, D, E, F, G, H) can be the same or different. The protease sequences (protease sequence A, B, C) can be the same or different.

Some embodiments provide a multispecific antibody of the disclosure with the addition of an immune checkpoint modulatory domain to enhance immune cell activity against said tumors. In some embodiments, the immunomodulatory domain is selected from a group consisting of a B7-1 polypeptide, a PD-L1 polypeptide, anti-PD-1 binding domain, and an anti-PD-L1 binding domain.

In some embodiments, the present disclosure provides a multispecific antibody comprising a binding arm that can target EGFR, a binding arm that can target cMet, and a binding arm that can target VEGF, wherein the EGFR arm, the cMET arm, and VEGF arm comprise amino acid sequences selected from SEQ ID NOs: 83-94, respectively. In some embodiments, the present disclosure provides a multispecific antibody comprising an amino acid sequence set forth in Table 10.

In some embodiments, the present disclosure provides a multispecific antibody comprising a binding arm that can target EGFR, wherein the binding arm comprises a human IgG1 Fab heavy chain sequence selected from SEQ ID NOs: 1 and 3, a light chain sequence selected from SEQ ID NOs: 2 and 4, and/or one or more single domain VHO sequences selected from SEQ ID NOs: 5-12, and/or one or more tandem single domain VHO sequences selected from SEQ ID NOs: 13-18.

In some embodiments, the present disclosure provides a multispecific antibody comprising a binding arm that can target cMET, wherein in the binding arm comprises a human IgG1 Fab heavy chain sequence selected from SEQ ID NOs: 23-24, 27-29, and 33-37, a light chain sequence selected from SEQ ID NOs: 25-26, 30-32, and 38-40, and/or one or more single domain VHO sequences selected from SEQ ID NOs: 41-44.

EGFR is recognized to interact with many other cell surface markers that contribute to development of cancers (Wang, Ma et al. 2015, Kennedy, Hastings et al. 2016). In some embodiments, the present disclosure provides a multispecific antibody that can effectively inhibit EGFR receptor association with a HER family receptor selected from HER2, HER3, HER4, and the corresponding downstream signaling factors. In some embodiments, the present disclosure provides a multispecific antibody comprising a binding arm that can target EGFR, wherein the binding arm comprises a human IgG1 Fab heavy chain sequence selected from SEQ ID NOs: 1 and 3 and light chain sequence selected from SEQ ID Nos: 2 and 4. In some embodiments, the present disclosure provides a multispecific antibody comprising a binding arm that can target EGFR, wherein the binding arm comprises one or more single domain VHO sequences selected from SEQ ID NOs: 5-18.

The heterodimerization of cMET with other receptors contribute to cancer development (Viticchie and Muller 2015). In some embodiments, the present disclosure provides a multispecific antibody that can effectively inhibit cMET receptor association with one or more of Plexin B1 family, CD44 family members, Integrin family receptors including α6β4, α5β1, α3β1, α2β1 and RTKs such as Ron, IGFR, RET, Death receptors such as Fas and DR5, and the corresponding downstream signaling factors. In some embodiments, the present disclosure provides a multispecific antibody comprising a binding arm that can target cMET, wherein the binding arm comprises a heavy chain sequence selected from SEQ ID NOs: 23-24, 27-29, 33-37, a light chain sequence selected from SEQ ID NOs: 25-26, 30-32, and 38-40. In some embodiments, the present disclosure provides a multispecific antibody comprising a binding arm that can target cMET, wherein the binding arm comprises one or more single domain VHO sequences selected from SEQ ID NOs: 41-44.

Some embodiments provide for a multispecific antibody that has one or more shielded epitopes, shielding, or caps (e.g., shield A, B, C, D) that can be removed by proteases and/or other in situ specific enzymes which are found in a tumor microenvironment. The presence of shielded epitopes is to minimize the systemic toxicity induced by the multispecific antibody.

In some embodiments, the present disclosure provides a multispecific antibody comprising one or more shielding or caps that mask cMET and/or EGFR mAb binding. In some embodiments, the shielding or cap for the antibodies is selected from SEQ ID NOs: 45-51. In some embodiments, the shielding or cap that masks cMET and/or EGFR single domain binding is selected from SEQ ID NOs: 52-61. In some embodiments, the present disclosure provides a multispecific antibody, wherein the shield or cap is attached via a protease substrate linker and optionally a peptide linker or spacer. The protease substrate linker is selected from SEQ ID NOs: 62-69.

In some embodiments, an antibody of the present disclosure (e.g., anti-EGFR, anti-cMET, anti-PD-L1, anti-VEGF, or multispecific antibody) can be a whole antibody, an antibody fragment, an antibody mimetic, a humanized antibody, a single chain antibody, an immunoconjugate, a defucosylated antibody, or a multispecific antibody. The antibody fragment may be selected from the group consisting of a UniBody, a domain antibody, and a VHO domain.

In some embodiments, an antibody or fragment of the present disclosure (e.g., anti-EGFR, anti-cMET, anti-PD-L1, anti-VEGF, or multispecific antibody) disclosed herein may be human, humanized, or chimeric antibodies or antigen binding fragments.

In some embodiments, an antibody of the present disclosure (e.g., anti-EGFR, anti-cMET, anti-PD-L1, anti-VEGF, or multispecific antibody) may be full length IgG1, IgG2, IgG3, or IgG4 antibodies or may be antigen-binding fragments thereof, such as a Fab, F(ab′)2, or scFv fragment. The antibody backbones may be modified to affect functionality, e.g., to eliminate residual effector functions.

In some embodiments, the disclosure provides an immunoconjugate comprising an antibody or fragment disclosed herein and a therapeutic agent. In some embodiments, the therapeutic agent carries a cytotoxin or a radioactive isotope.

Some embodiments provide for a multispecific antibody that can be in a human IgG1, IgG2, IgG3, and/or IgG4 framework. The multispecific antibody can be engineered to have a hinge region with enhanced protease stability. The multispecific antibody can also be engineered to have a shorter and longer half-life.

In some embodiments, the heavy chain of a multispecific antibody comprises a constant region of an IgG1 antibody, preferably a human IgG1 antibody. The CH2 region of said IgG1 constant region can be engineered to alter ADCC, ADCP, and/or CDC activity of the antibody. In a preferred embodiment, said alteration results in enhanced ADCC (Antibody-dependent cellular cytotoxicity), ADCP (antibody-dependent cellular phagocytosis), and/or CDC activity. In a preferred embodiment, the CH3-region of the multispecific antibody is engineered to facilitate heterodimerization of heavy chains comprising a first heavy chain that binds EGFR and a second heavy chain that binds cMET.

In one embodiment, the multispecific antibody can induce higher levels of downmodulation of EGFR and cMET when compared to their parental mAbs. This multispecific antibody activity can result in decreasing the viability of the tumor cells that are driven by EGFR and cMET signaling cascades.

Some embodiments provide a multispecific antibody that targets and binds to human EGFR and cMET simultaneously, has high affinity, and is capable of effectively blocking EGFR at the protein level. The multispecific antibody binds both cMET and EGFR proteins or binds to one target protein without affecting the binding of another target protein, having the ability to bind cMET and EGFR simultaneously. The multispecific antibody inhibits the proliferation of vascular endothelial cells, human lung cancer cells, human breast cancer cells, human pancreatic cancer cells, and/or human gastric cancer cells.

In some embodiments, the present disclosure provides a composition comprising an antibody or fragment of the present disclosure (e.g., anti-EGFR, anti-cMET, anti-PD-L1, anti-VEGF, or multispecific antibody) and a carrier.

In some embodiments, the present disclosure provides a pharmaceutical composition comprising an antibody or fragment of the present disclosure (e.g., anti-EGFR, anti-cMET, anti-PD-L1, anti-VEGF, or multispecific antibody) and a pharmaceutically acceptable carrier.

Some embodiments provide a composition comprising a multispecific antibody disclosed herein. In some embodiments, the multispecific antibody can be present at a concentration of 10 mg/mL to 250 mg/mL in the composition.

In some embodiments, the composition of the present disclosure further comprises at least one buffer, at least one stabilizer, and/or at least one surfactant. In some embodiments, the composition is liquid. In some embodiments, the composition is formulated for subcutaneous injection. In some embodiments, the composition is sterile. In some embodiments, the composition further comprises histidine HCl, trehalose dehydrate, methionine and/or polysorbate.

In some embodiments, the present disclosure provides a method for treating or preventing a disease associated with target cells expressing cMET, EGFR, and PD-L1/VEGF, comprising administering to a subject an antibody of the disclosure, such as, an anti-cMET×anti-EGFR×PD-L1/VEGF multispecific antibody, or antigen binding portion thereof, in an amount effective to treat or prevent the disease. In some examples, the disease treated or prevented is human cancer. In some examples, the diseases treated or prevented include lung cancer, head and neck cancer, colorectal cancer, gastric cancer, breast, intestinal cancer, neuroendocrine, glioblastoma multiforme, and pancreatic cancer.

In some embodiments, the disclosure provides an antibody of the disclosure, e.g., an anti-cMET×anti-EGFR×PD-L1/VEGF multispecific antibody, or an antigen-binding portion thereof, for use in treating or preventing a cancer associated with target cells expressing cMET, PD-L1/VEGF, and EGFR. In some embodiments, the disease treated or prevented is human cancer. In some embodiments, the diseases treated or prevented include lung cancer, head and neck cancer, colorectal cancer, gastric cancer, intestinal cancer, neuroendocrine, glioblastoma multiforme, breast, and pancreatic cancer.

In some embodiments, the disclosure provides the use of an antibody of the disclosure, e.g., an anti-cMET×anti-EGFR×PD-L1/VEGF multispecific antibody, or antigen-binding portion thereof, for the manufacture of a medicament for use in treating or preventing a cancer associated with target cells expressing cMET, PD-L1/VEGF, and EGFR. In some embodiments, the disease treated or prevented by the medicament of the disclosure is human cancer. In some embodiments, the diseases treated or prevented by the medicament of the present disclosure includes lung cancer, head and neck cancer, colorectal cancer, gastric cancer, intestinal cancer, neuroendocrine, breast, glioblastoma multiforme, and pancreatic cancer.

In some embodiments, the type of the cancer to be treated or prevented by an antibody or fragment of the disclosure can be conventional cancer, preferably, selected from lung cancer, breast cancer, pancreatic cancer, and gastric cancer.

Some embodiments provide for a multispecific antibody that can be developed into combination regimens using higher doses of chemotherapy with EGFR inhibitors to determine the best synergistic partners.

Some embodiments provide for a multispecific antibody that can be developed into combination regimens using higher doses of chemotherapy with cMET inhibitors to determine the best synergistic partners.

Some embodiments provide a multispecific antibody of the disclosure that may be used to treat a tumor which is resistant to an EGFR tyrosine kinase inhibitor, including for example, but not limited to, erlotinib, gefitinib, Osimertinib, dacomitinib, or afatinib, an analogue of erlotinib, gefitinib, Osimertinib, dacomitinib, or afatinib, or a combination of one or more of the respective compounds and/or analogues thereof.

Some embodiments provide a multispecific antibody of the disclosure that may be used to treat a tumor which is resistant to treatment with an cMET tyrosine kinase inhibitor, including for example, but not limited to, crizotinib, cabozantinib, tivantinib, teptotinib, an analogue of crizotinib, cabozantinib, tivantinib, teptotinib, or a combination of one or more of the respective compounds and/or analogues thereof.

In some embodiments, the present disclosure provides an isolated nucleic acid molecule encoding the heavy or light chain of an antibody or antigen binding portion thereof of the disclosure. In some embodiments, the present disclosure provides an expression vector comprising one or more of such nucleic acids, and a host cell comprising one or more of such expression vectors.

In some embodiments, the present disclosure provides a hybridoma expressing an antibody or antigen binding portion of the disclosure.

In some embodiments, the disclosure provides an isolated nucleic acid molecule encoding the heavy or light chain of an isolated multispecific antibody or antigen-binding portion which binds epitopes on human EGFR and cMET and PD-L1 or VEGF. In some embodiments, the disclosure provides expression vectors comprising such nucleic acid molecules, and host cells comprising such expression vectors.

Some embodiments provide a method for producing a multispecific antibody disclosed herein comprising the steps of culturing a recombinant expression transformant disclosed herein and obtaining the multispecific antibody from the culture.

Some embodiments provide the application of a multispecific antibody disclosed herein in the manufacture of a medicament for the treatment or prevention of cancer.

Some embodiments provide a nucleic acid encoding a multispecific antibody targeting cMET with a mask and targeting EGFR with another mask.

In some embodiments, the present disclosure provides a method for preparing an anti-cMET×anti-EGFR×PD-L1/VEGF multispecific antibody, said method comprising: obtaining a host cell that contains one or more nucleic acid molecules encoding the antibody of the disclosure; growing the host cell in a host cell culture; providing host cell culture conditions wherein the one or more nucleic acid molecules are expressed; and recovering the antibody from the host cell or from the host cell culture.

In some embodiments, the present disclosure provides cDNA that encodes an isolated multispecific antibody, an antigen binding portion, an antibody fragment, or a multispecific antibody mimetic. In some embodiments, the present disclosure provides expressing said cDNA in phages such that the multispecific antibody, the antigen binding portion thereof, the antibody fragment, or the multispecific antibody mimetic (e.g., anti-cMET, anti-PD-L1/VEGF, and anti-EGFR multispecific antibodies) encoded by said cDNA are presented on the surface of said phages; selecting phages that present the multispecific antibody, the antigen binding portion, the antibody fragment, or the multispecific antibody mimetic; recovering nucleic acid molecules from said selected phages that encode the multispecific antibody, the antigen binding portion, the antibody fragment, or the multispecific antibody mimetic; expressing said recovered nucleic acid molecules in a host cell; and recovering the multispecific antibody, the antigen binding portion, the antibody fragment, or the multispecific antibody mimetic from said host cell.

In some embodiments, the present disclosure provides a method for producing a multispecific antibody disclosed herein. The recombinant DNA encoding the parental antibodies for the multispecific antibody is prepared by the DNA recombination techniques and then transfected into mammalian cells to express the parental antibodies. After purification, identification, and screening, the multispecific antibody is generated using the controlled Fab arm exchange or other multispecific antibody generation process to generate a multispecific antibody which shows the biological effects of simultaneous binding to, e.g., EGFR and cMET. The multispecific antibody affinity and blocking efficiency are identified through the completion of in vitro experiments.

These and other embodiments of the present disclosure will be described in greater detail herein.

### BRIEF DESCRIPTION OF THE FIGURES

FIG. 1 shows the Profile of inhibitors of EGFR and cMET signaling pathways in cancer. The binding of EGF to EGFR and HGF to cMET leads to phosphorylation of specific tyrosine residues and subsequent activation of these receptors. Overexpression of EGFR and cMET RTKs in certain cancers results in activation of downstream signaling pathways PI3K/Akt and MAPK (RAS-RAF, MEK-ERK/MAPK). The induction of these signaling cascades results in the stimulation of cancer cell survival through dysregulation of cell death pathways. Several inhibitors inhibit these pathways by binding to the tyrosine kinase domains or to ligands resulting in receptor inactivation. The TKIs and mAbs of the EGFR and cMET signaling pathways are shown in boxes with their targets marked by inhibitory or activation arrows as indicated in the figure.

FIG. 2 shows that 7D VH hits bind to EGFR and block EGFR-EGF binding using ELISA. FIG. 2A shows that the 7D VH hits (7D VH1, 7D VH2, 7D VH3, 7D VH4, 7D VH5, and 7D VH6) bound to an EGFR extracellular domain (ECD) in an ELISA format. There was no binding by a gp120 mAb. Cetuximab and the 7D VHO hits bound to the EGFR ECD in the ELISA format. In this EGFR binding experiment, the EC50 values in units of ng/mL were Cetuximab ˜10 ng/ml; 7D VH1 (Fv noted in SEQ ID NO: 5)˜14 ng/ml; 7D VH2 (Fv noted in SEQ ID NO: 6)˜3 ng/ML; 7D VH3 (Fv noted in SEQ ID NO: 7)˜3 ng/ml; 7D VH4 (Fv noted in SEQ ID NO: 8)˜3 ng/ml; 7D VH5 (Fv noted in SEQ ID NO: 9)˜3 ng/ml; and 7D VH6 (Fv noted in SEQ ID NO. 10)˜2 ng/mL. FIG. 2B shows the binding of TAVO412E (also referred to as “TAVO412” herein) to recombinant human EGFR ECD in the ELISA format with an EC50 value of 0.059 nM. FIG. 2C shows the binding of TAVO412E to recombinant cynomolgus monkey EGFR ECD in the ELISA format with an EC50 value of 0.109 nM. In FIGS. 2A, 2B, and 2C, the y axes represented the absorbance at 450 nm that reflected the ELISA binding levels and the x axes represented the concentration of the test reagents.

FIG. 2D shows that the 7D VH hits blocked EGFR ECD from binding to EGF in HCC827 cells. There was no blocking by the gp120 mAb. In this EGF ligand-EGFR binding blocking experiment, the EC50 values are (in units of ng/ml): Cetuximab ˜65 ng/ml; 7D VH1 (Fv noted in SEQ ID NO: 5)˜25 ng/ml; 7D VH2 (Fv noted in SEQ ID NO: 6)˜23 ng/ml; 7D VH3 (Fv noted in SEQ ID NO: 7)˜31 ng/mL; 7D VH4 (Fv noted in SEQ ID NO: 8)˜38 ng/ml; 7D VH5 (Fv noted in SEQ ID NO: 9)˜34 ng/ml; and 7D VH6 (Fv noted in SEQ ID NO: 10)˜36 ng/mL. In FIG. 2D, the y axis represented the geometric mean fluorescence intensity (gMFI) that reflected the binding level on cells and the x axis represented the concentration of the test reagents. FIG. 2E shows that TAVO412E blocked EGFR ECD from binding to EGF in an ELISA format with an IC50 value of 1.53 nM. In FIG. 2E, the y axis represented the absorbance at 450 nm that reflected the binding level of EGF to EGFR via ELISA and the x axis represented the concentration of the test reagents.

FIG. 3 shows that cMET hits bind to cMET and block cMET-HGF binding using ELISA. In FIG. 3A-3G, the y axes represented the absorbance at 450 nm that reflected the ELISA binding levels and the x axes represented the concentration of the test reagents. FIG. 3A shows the cMET hits bound to the cMET extracellular domain (ECD) in an ELISA format. The test articles and sequence information are presented in the table below. The amivantamab analogue uses the JNJ-61186372 heavy and light chain amino acid sequences with relevant low fucosylation. The terms amivantamab, amivantamab analogue, JNJ-61186372, or JNJ-6372 all refer to the same amino acid sequences with the same relevant low fucosylation.

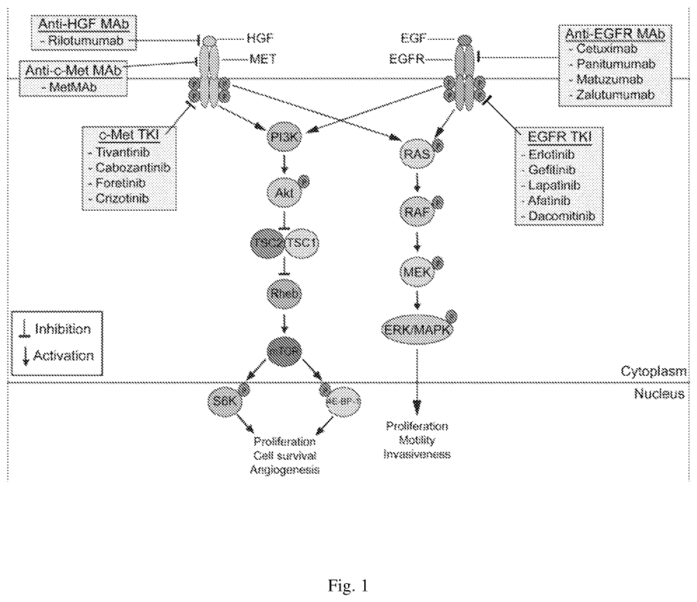

| Test article name | Sequence Information |
|---|---|
| met 4K v1 | mAb with HC Fv SEQ ID NO: 34 and LC Fv |
| | SEQ ID NO: 39 |
| met 4K v2 | mAb with HC Fv SEQ ID NO: 34 and LC Fv |
| | SEQ ID NO: 40 |
| met 4K v3 | mAb with HC Fv SEQ ID NO: 35 and LC Fv |
| | SEQ ID NO: 39 |
| met 4K v4 | mAb with HC Fv SEQ ID NO: 35 and LC Fv |
| | SEQ ID NO: 40 |
| met 4K v5 | mAb with HC Fv SEQ ID NO: 36 and LC Fv |
| | SEQ ID NO: 39 |
| met 4K v6 | mAb with HC Fv SEQ ID NO: 36 and LC Fv |
| | SEQ ID NO: 40 |
| met 4K v7 | mAb with HC Fv SEQ ID NO: 37 and LC Fv |
| | SEQ ID NO: 39 |
| Onartuzumab | mAb with HC Fv SEQ ID NO: 33 and LC Fv |
| | SEQ ID NO: 38 |
| gp120 (inert arm) × | Bispecific Ab comprising mAb with HC Fv |
| JNJ cMET binding arm | SEQ ID NO: 82 and LC Fv SEQ ID NO: 81 |
| | and mAb with HC Fv SEQ ID NO: 77 and |
| | LC Fv SEQ ID NO: 78 |
| amivantamab cMet | mAb with HC Fv SEQ ID NO.: 77 and LC Fv |
| | SEQ ID NO: 78 |

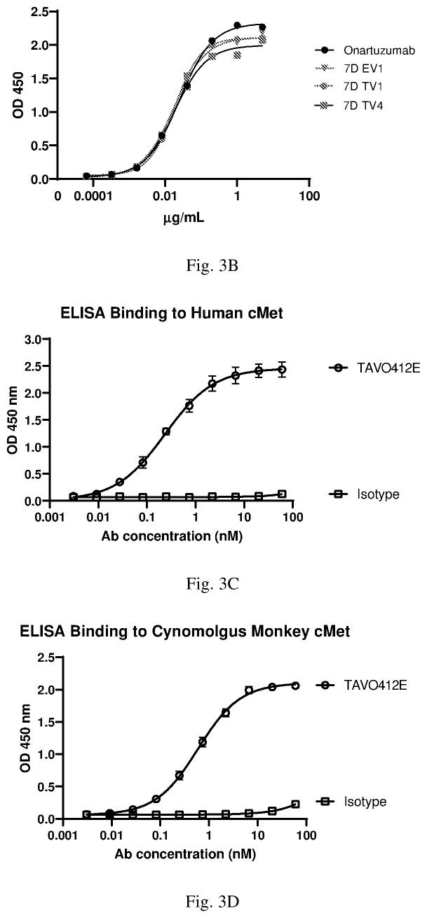

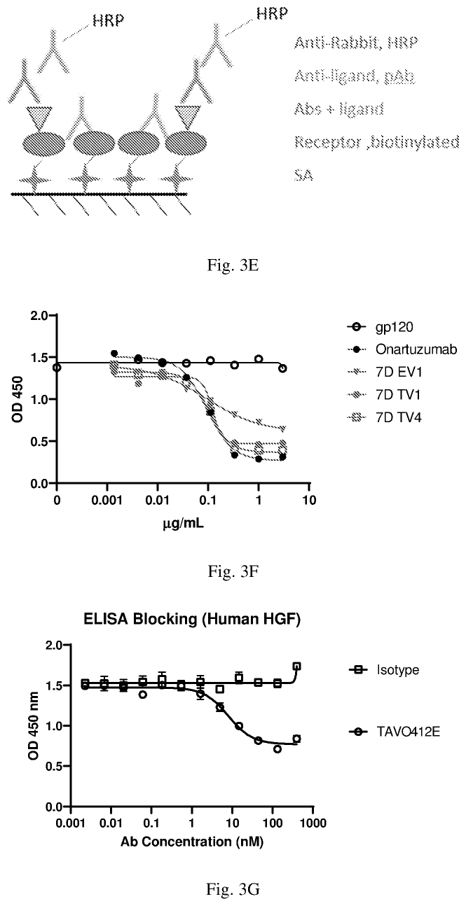

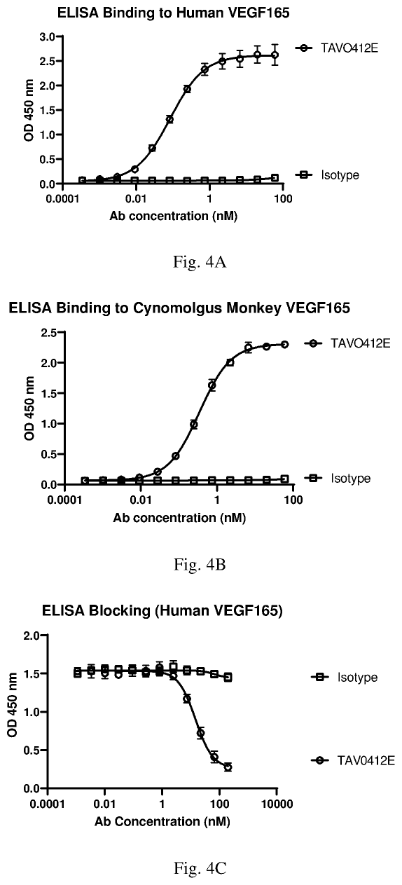

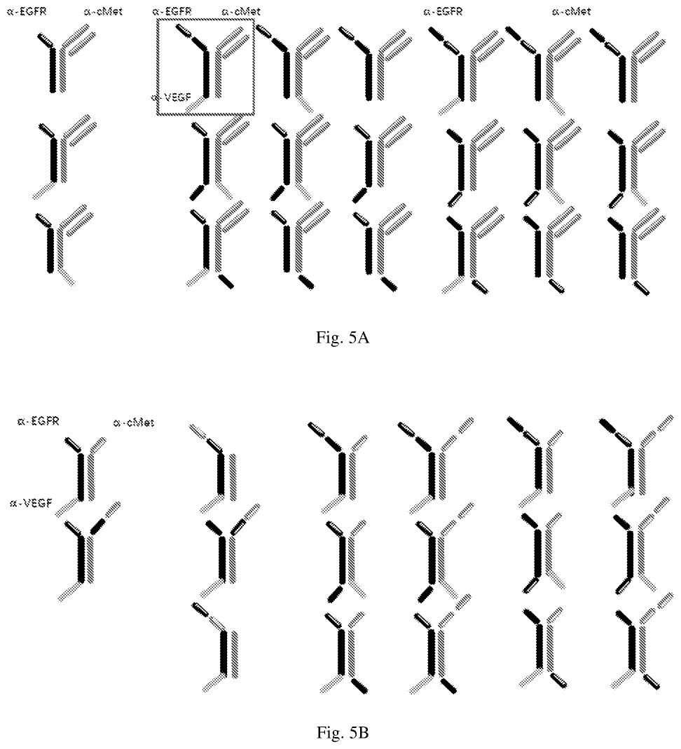

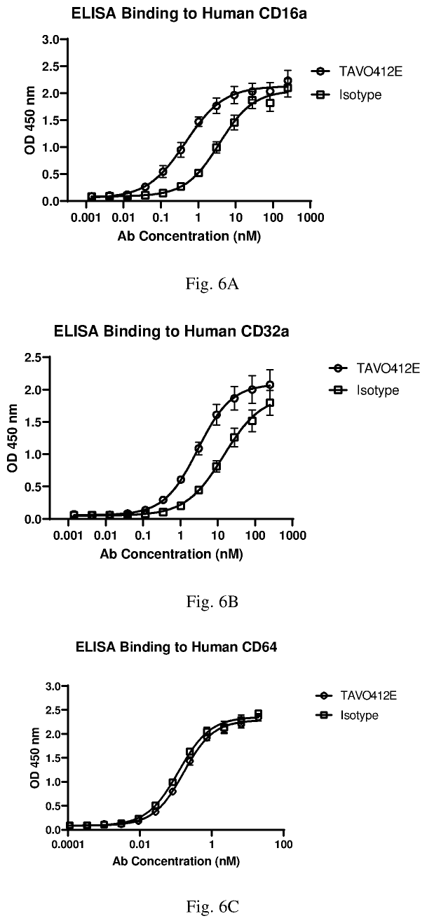

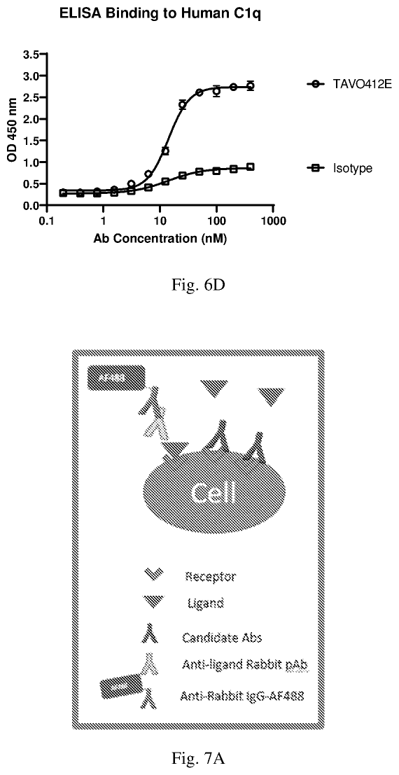

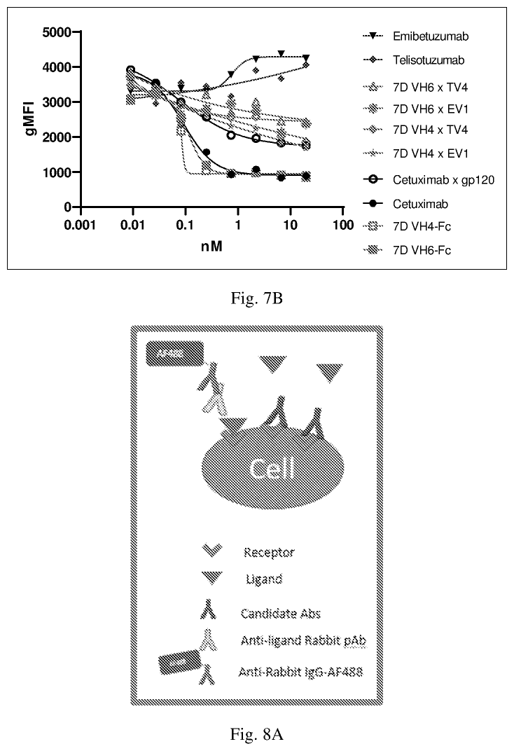

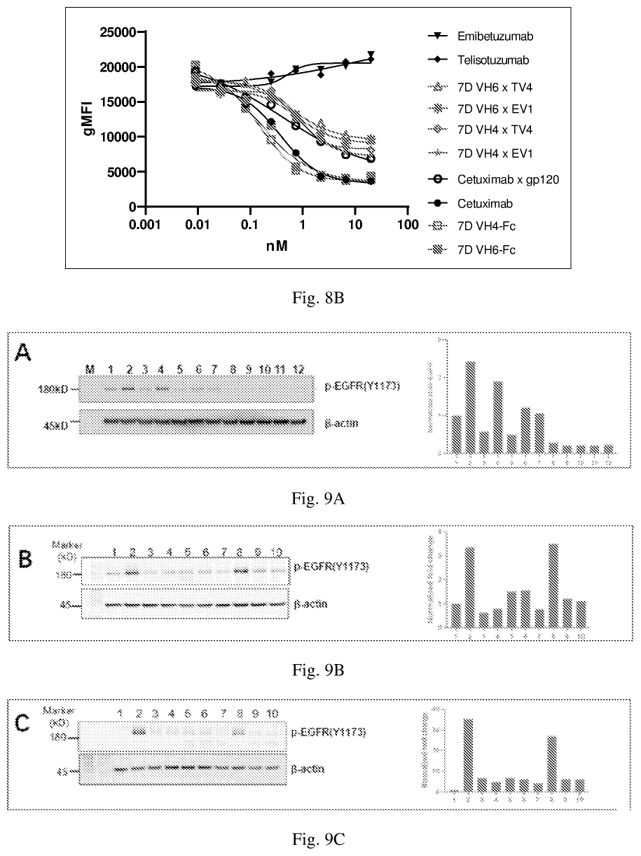

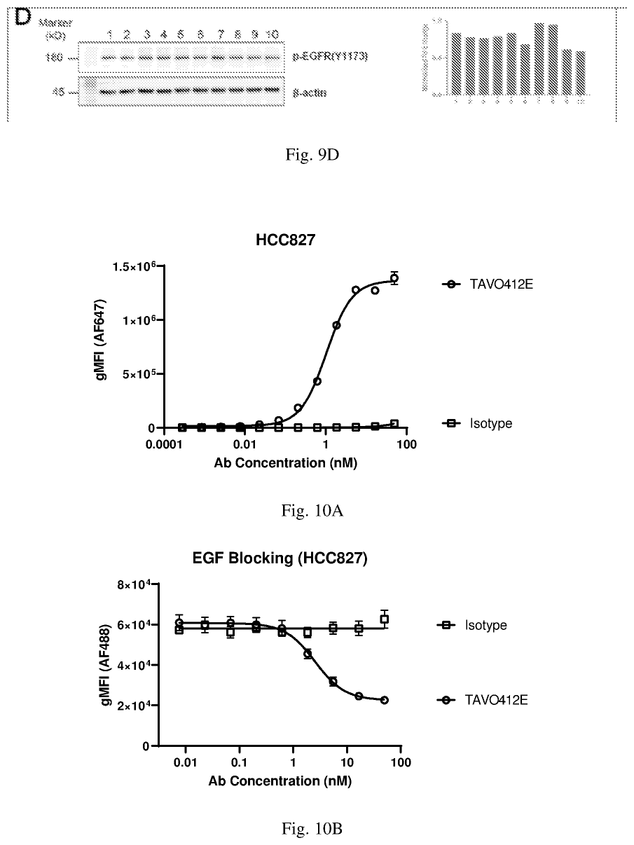

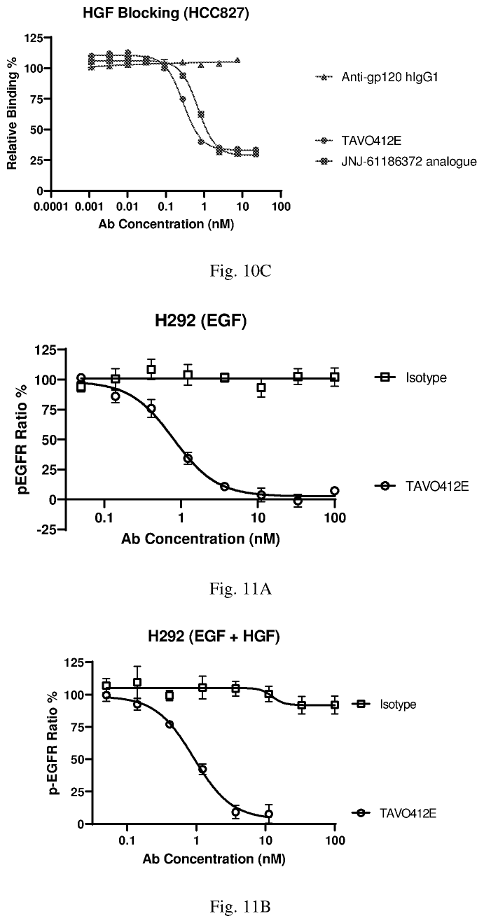

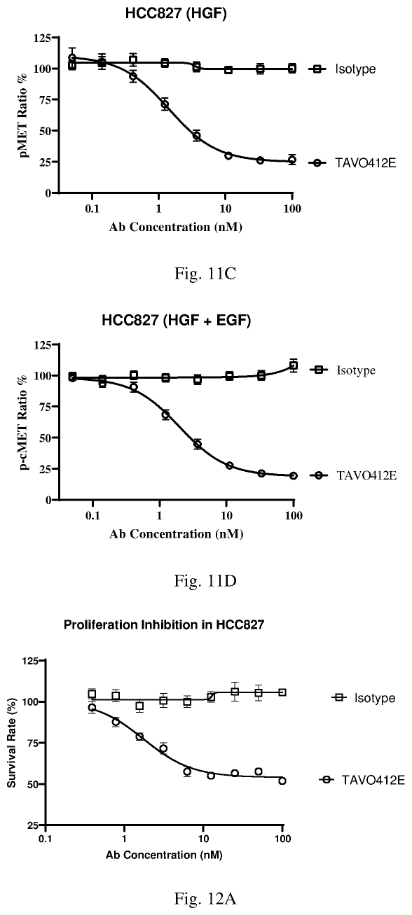

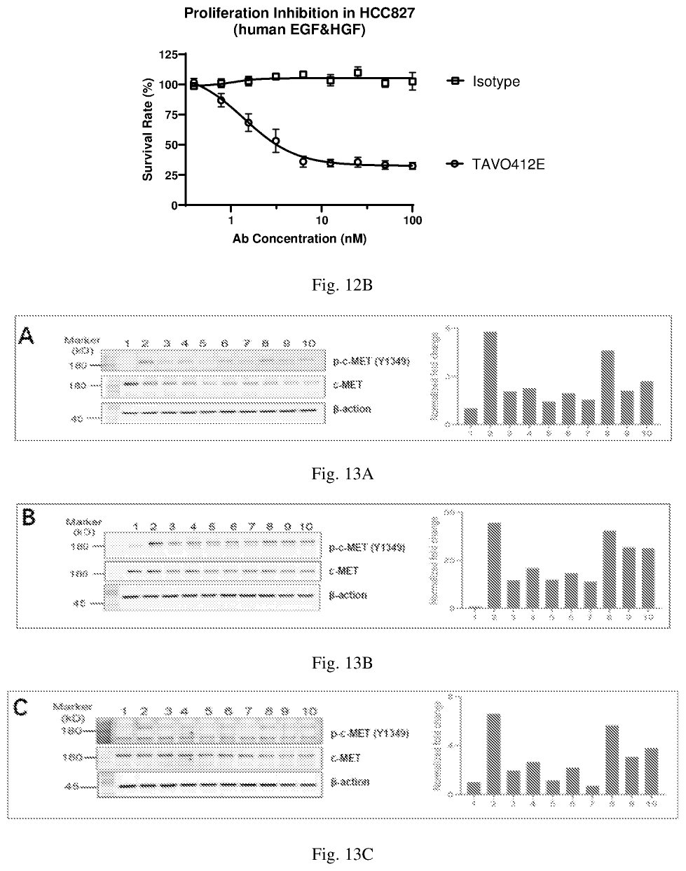

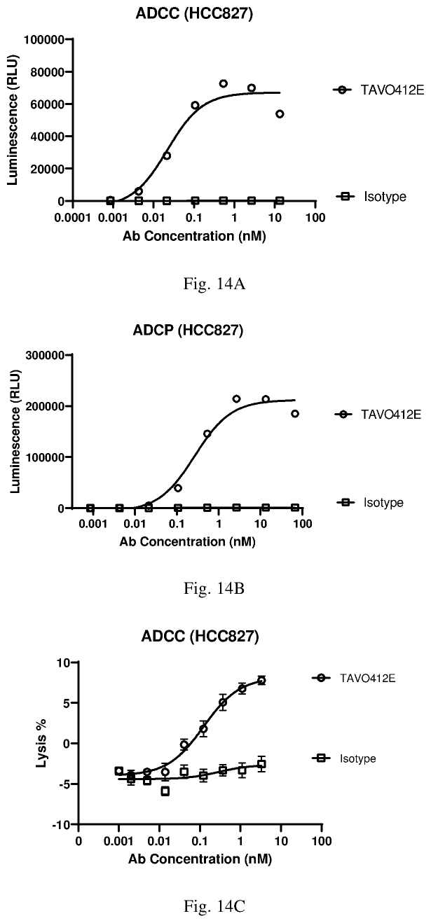

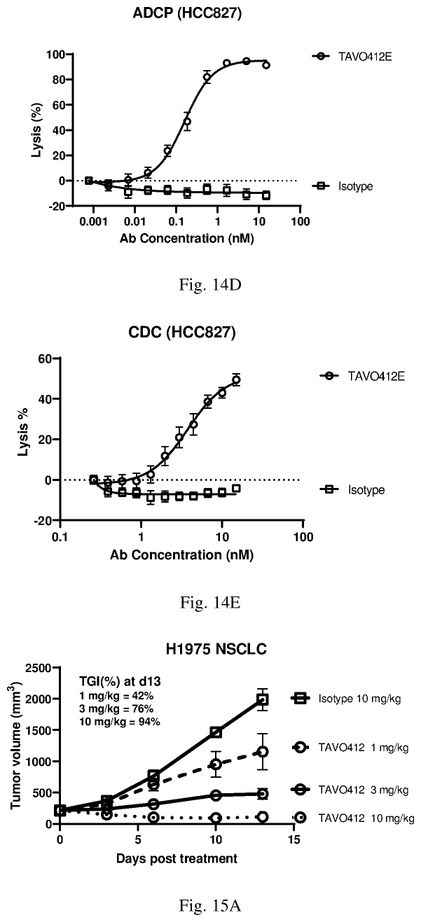

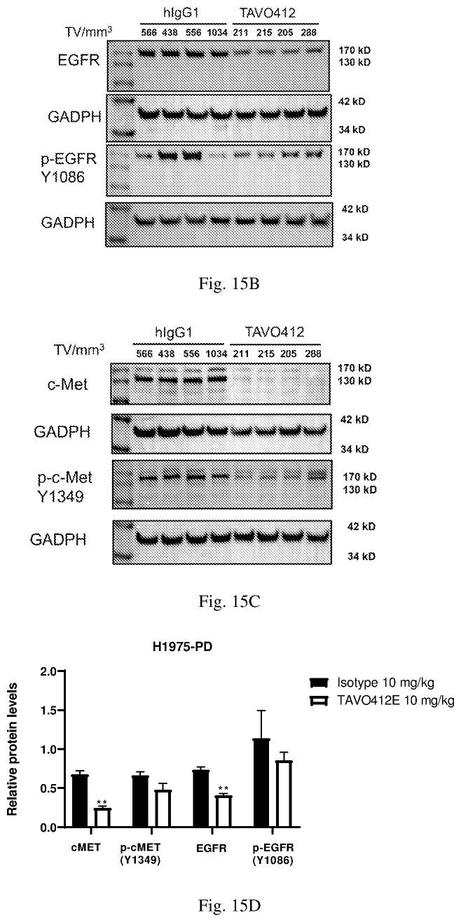

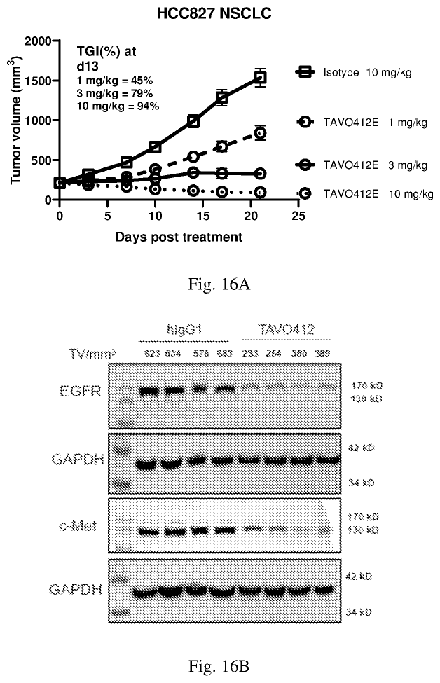

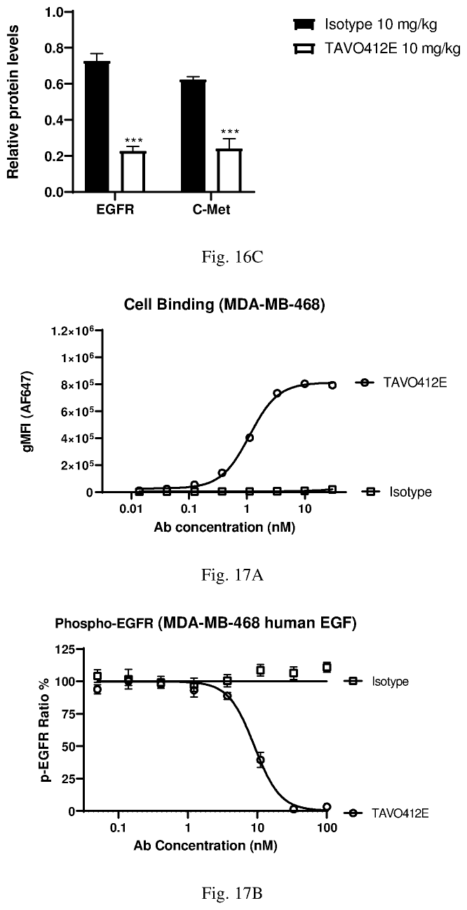

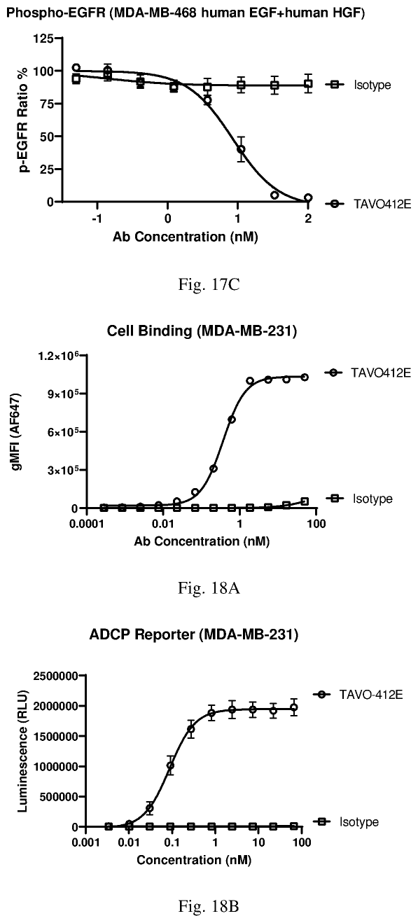

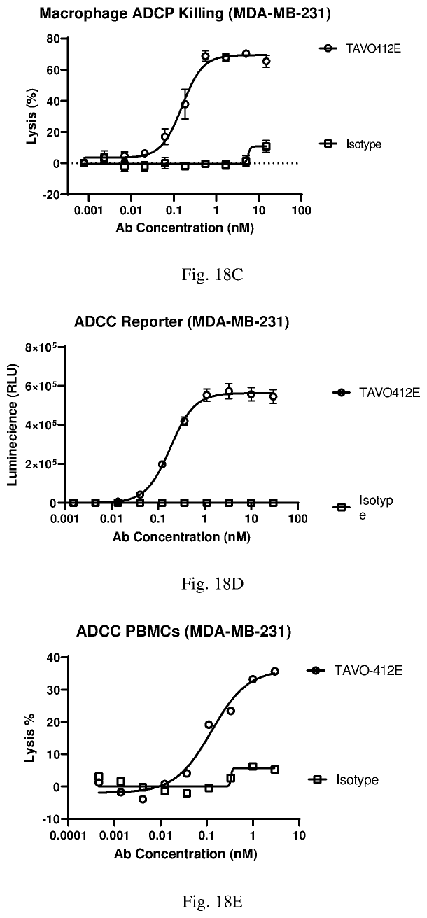

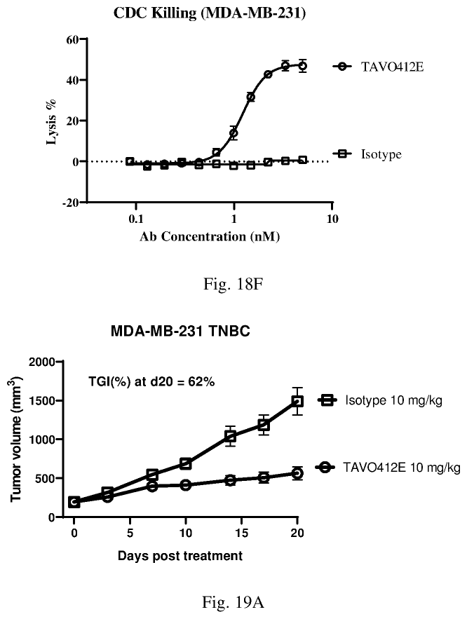

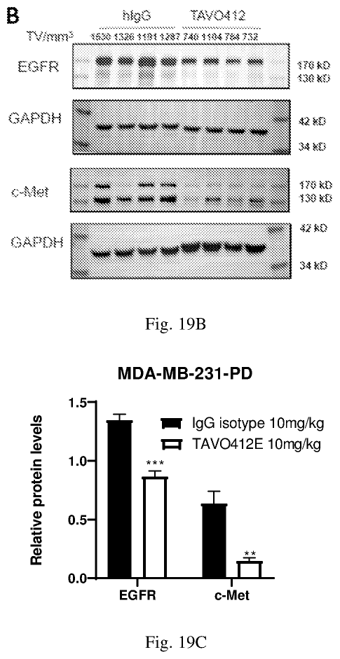

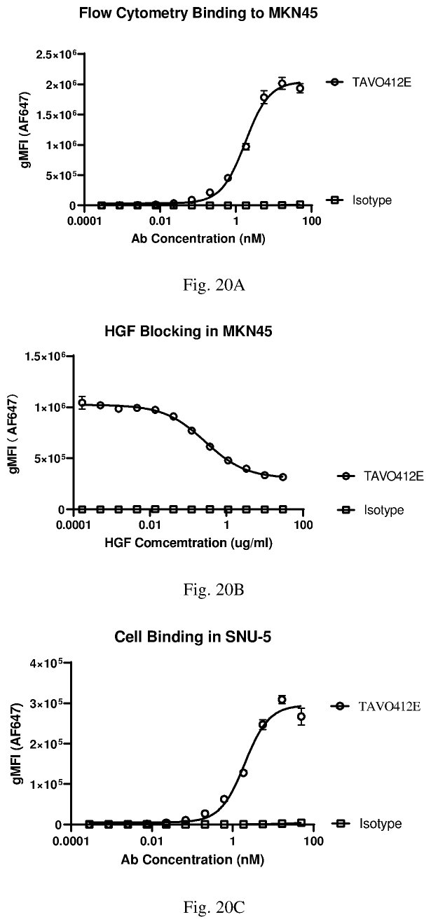

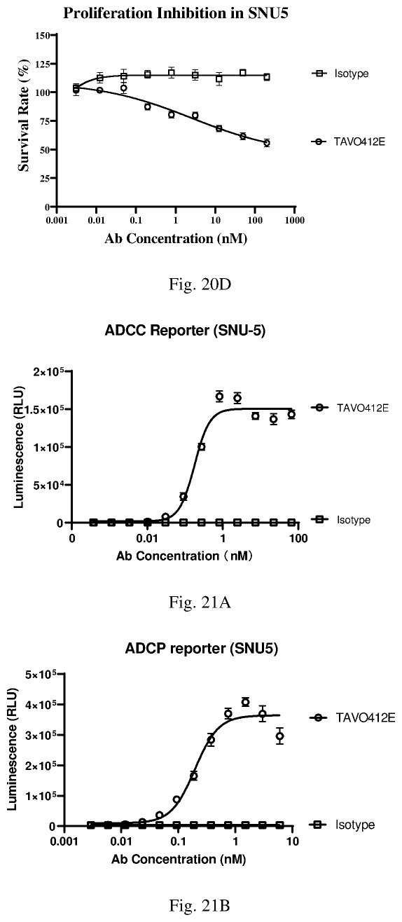

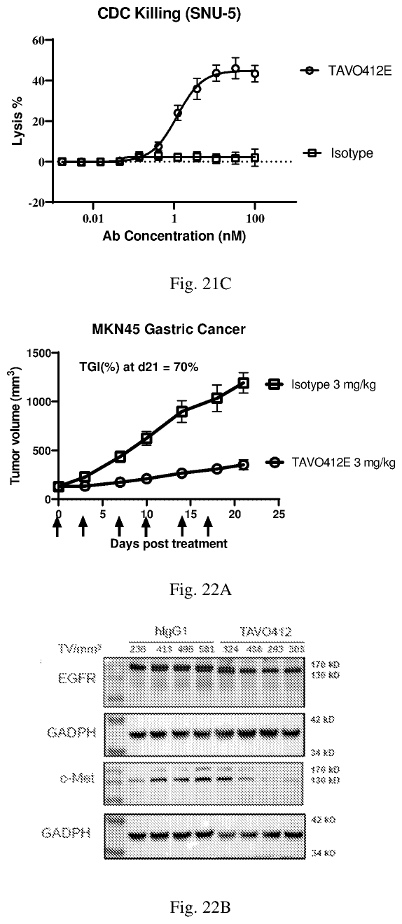

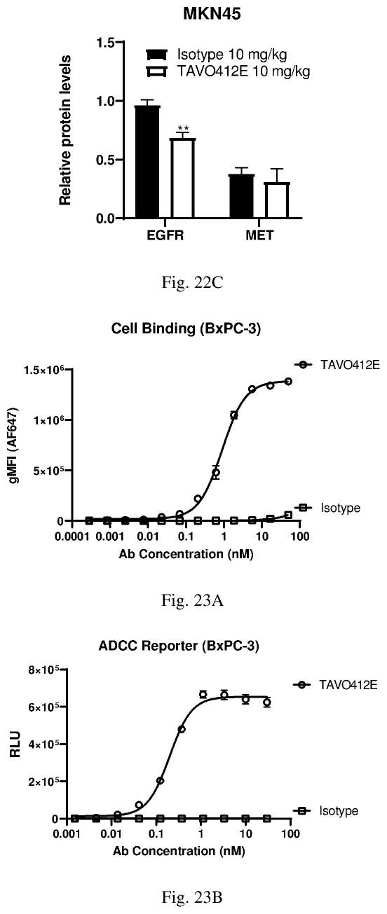

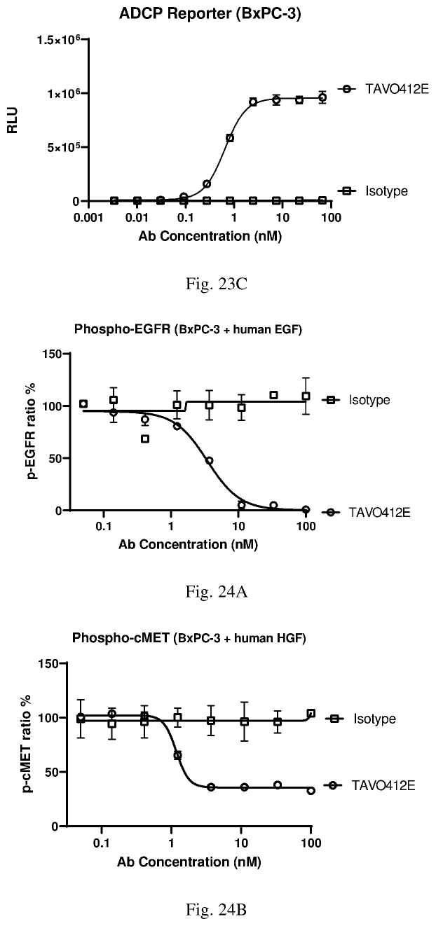

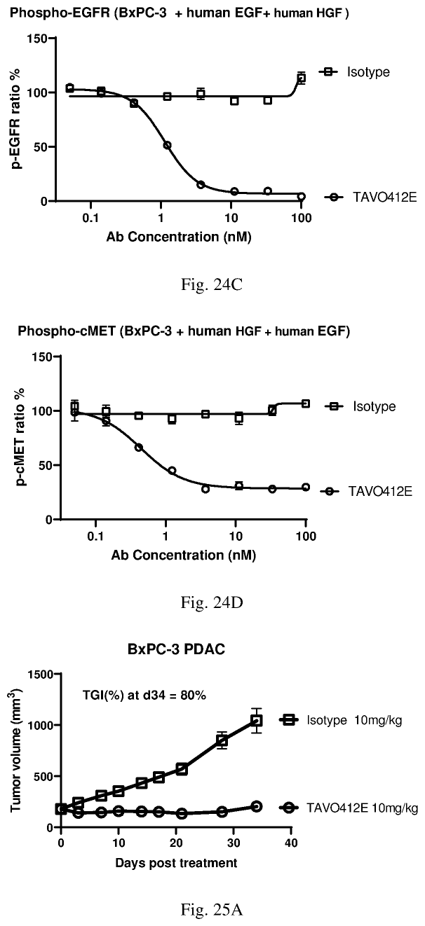

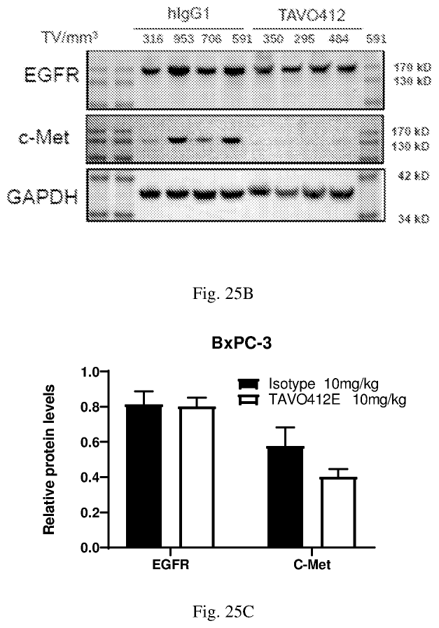

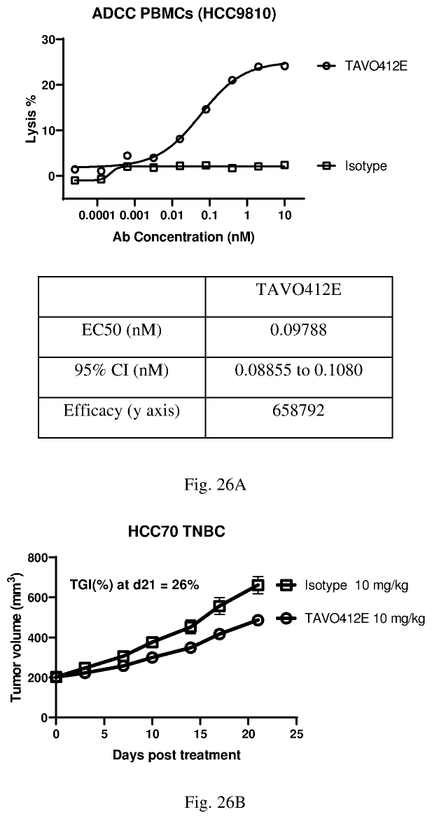

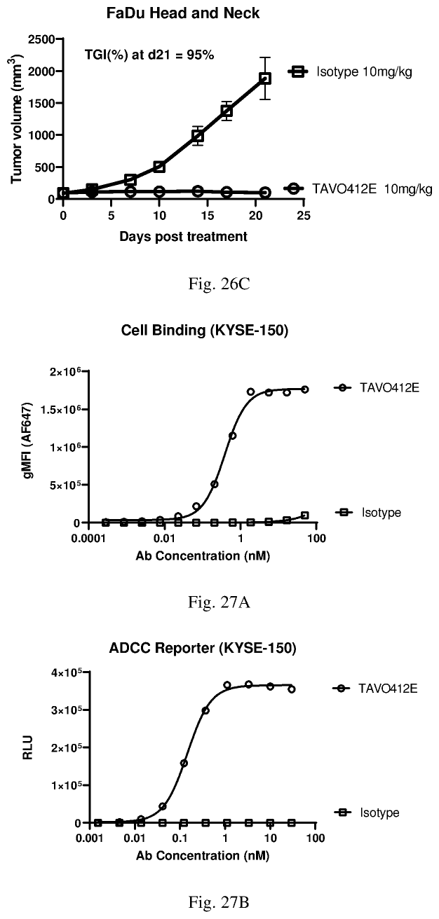

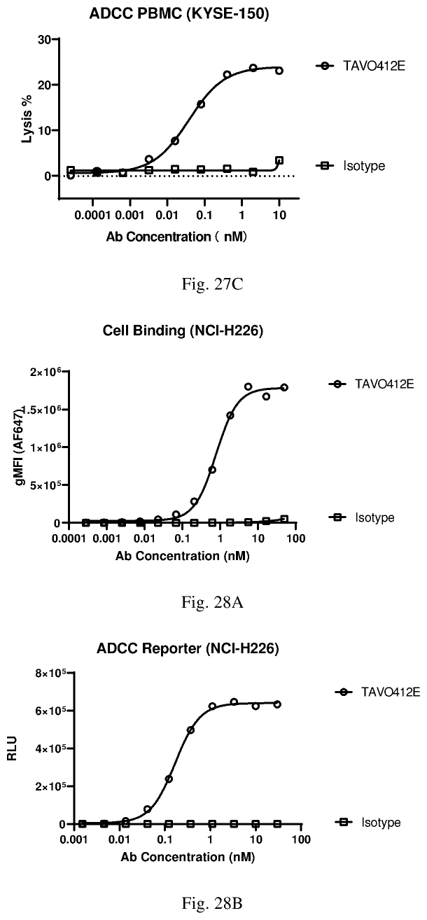

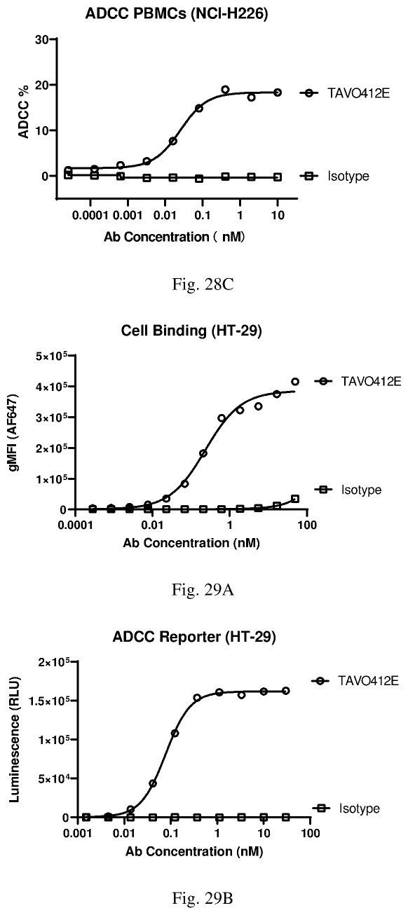

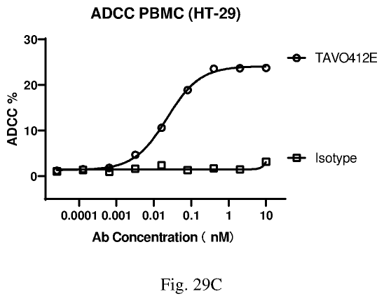

FIG. 3A shows Onartuzumab and EV1 (heavy chain Fv noted in SEQ ID NO: 24, light chain Fv noted in SEQ ID NO: 26), TV1 (heavy chain Fv noted in SEQ ID NO: 28, light chain Fv noted in SEQ ID NO: 30), TV4 (heavy chain Fv noted in SEQ ID NO: 29, light chain Fv noted in SEQ ID NO: 32) hits bound to the cMET ECD in an ELISA assay. In this cMET binding experiment, the EC50 values in units of ng/mL were: Onartuzumab ˜25 ng/mL; EV1˜17.5 ng/ml; TV1˜18.5 ng/mL; and TV4˜18.0 ng/mL. FIG. 3B shows that TAVO412E has potent binding to recombinant human cMET with an EC50 value of 0.234 nM. There was no binding to human cMET by the isotype mAb. FIG. 3C shows that TAVO412E had potent binding to recombinant cynomolgus monkey cMET with an EC50 value of 0.595 nM. There was no binding to the cynomolgus monkey cMET by the isotype mAb. The schematic of the ligand blocking assay is shown in FIG. 3E. Streptavidin was coated on an ELISA plate. Subsequently, the biotinylated cMET ECD was added to this layer. The antibodies and ligand were then added to compete for binding to the cMET ECD. The primary antibody was a rabbit polyclonal anti-HGF Ab and the secondary antibody was a HRP labeled anti-rabbit antibody for detection. The assay format can be reformatted to be used in a cMET-HGF blocking assay as will be described later.

FIG. 3F shows that the cMET hits blocked cMET ECD from binding to HGF in an ELISA format. There was no blocking by the gp120 mAb. In this cMET-HGF blocking binding experiment, the EC50 values in units of ng/mL were: Onartuzumab ˜93 ng/ml; EV1˜93 ng/mL; TV1˜122 ng/ML; TV4˜108 ng/mL. FIG. 3G shows that TAVO412E blocked the binding of Human hepatocyte growth factor (HGF) to recombinant human cMET with an IC50 value of 8.05 nM.

FIG. 4 shows TAVO412E binds to VEGF and block VEGF-VEGFR binding using ELISA. In FIG. 4A-C, the y axes represented the absorbance at 450 nm that reflected the ELISA binding levels and the x axes represented the concentration of the test reagents. FIG. 4A shows that TAVO412E bound to a recombinant human VEGF165 in an ELISA format with an EC50 value of 0.084 nM. There was no binding by the isotype mAb. FIG. 4B shows the binding of TAVO412E to recombinant cynomolgus monkey VEGF165 in the ELISA format with an EC50 value of 0.346 nM. FIG. 4C shows that TAVO412E blocked the binding of recombinant human VEGF165 to recombinant human VEGFR with an IC50 value of 14.8 nM.

FIG. 5 shows structural designs for an anti-cMET×anti-EGFR multispecific antibody. The anti-cMET×anti-EGFR multispecific antibody is illustrated to show the EGFR binding arms in black, the cMET binding arms in dark grey, and the VEGF binding arms in light grey as indicated in the figure. FIG. 5A shows the EGFR binding arms can have a valency of one or two VHO domains. The cMET binding arm can have a valency of one Fab domain. The VEGF binding arm can have a valency of 1-2 domains. The EGFR VHO domains can be on the same heavy chain as N-terminal or C-terminal fusions of the Fc, as tandem Fc fusion molecules on the Fc, or as C terminal fusions on the cMET heavy chain. FIG. 5B shows the EGFR binding arms can have a valency of one or two VHO domains. The cMET binding arm can have a valency of one or two VHO domains on a FC domain. The VEGF binding arm can have a valency of 1-2 domains. The EGFR VHO domains can be on the same heavy chain as N-terminal or C-terminal fusions of the Fc, as tandem fusion molecules on the Fc, or as C-terminal fusions on the cMET VHO heavy chain fusion. The cMET VHO domains can be on the same heavy chain as N-terminal fusions of the Fc, as tandem fusion molecules on the Fc, or as N terminal fusions on the EGFR VHO heavy chain fusion molecules.

FIG. 6 shows TAVO412E binding to CD16a, CD32a, CD64, and C1q. In FIG. 6A-D, the y axes represented the absorbance at 450 nm that reflected the ELISA binding levels and the x axes represented the concentration of the test reagents. FIG. 6A shows that TAVO412E bound to a recombinant human CD16a in an ELISA format with an EC50 value of 0.46 nM as compared to an isotype mAb with human IgG1 with an EC50 value of 3.7 nM. FIG. 6B shows that TAVO412E bound to a recombinant human CD32a in an ELISA format with an EC50 value of 2.9 nM as compared to an isotype mAb with human IgG1 with an EC50 value of 14.0 nM. FIG. 6C shows that TAVO412E bound to a recombinant human CD64 in an ELISA format with an EC50 value of 0.16 nM as compared to an isotype mAb with human IgG1 with an EC50 value of 0.12 nM. FIG. 6D shows that TAVO412E bound to a recombinant human C1q in an ELISA format with an EC50 value of 14.2 nM as compared to an isotype mAb with human IgG1 with an EC50 value of 14.1 nM.

FIG. 7 shows inhibition of EGF ligand binding to EGFR in H292 cells. FIG. 7A shows the assay format of a FACS based assay that was used to characterize the ligand blocking of H292 cells (EGFR: cMET ratio of 365000 to 64000). An anti-cMET×anti-EGFR multispecific antibody was added to compete with 0.2 μg/mL EGF from binding to the cells. The EGF was detected using a AF488 nm labeled rabbit anti-EGF antibody. FIG. 7B shows that the gMFI was measured to determine the levels of EGF binding in the presence of the competing mAbs. In FIG. 7B, the y axis represented the gMFI that reflected the binding levels on H292 cells, and the x axis represented the concentration of the test reagents. In this assay, the competitor cMET antibodies do not have much effect on EGF binding to the H292 cell lines. The EGFR antibodies do bind and compete with EGF for binding to EGFR.

FIG. 8 shows inhibition of EGF ligand binding to EGFR in HCC827 cells. FIG. 8A shows the assay format of a FACS based assay that was used to characterize the ligand blocking of HCC827 cells (EGFR: cMET ratio of 420000 to 204000). FIG. 8B shows that the gMFI was measured to determine the levels of EGF binding in the presence of the competing mAbs. In FIG. 8B, the y axis represented the gMFI that reflected the binding levels on HCC827 cells, and the x axis represented the concentration of the test reagents. In this assay, the competitor cMET antibodies do not have much effect on EGF binding to the HCC827 cell lines. The EGFR antibodies do bind and compete with EGF for binding to EGFR. In this EGFR-EGF blocking experiment, the EC50 values in units of ng/ml for the EGFR×cMet hits were 7D VH6×TV4˜0.63 nM; 7D VH6×EV1˜0.63 nM; 7D VH4×TV4˜0.93 nM; 7D VH4×EV1˜0.81 nM; cetuximab×gp120˜0.60 nM; cetuximab ˜0.33 nM; 7D VH4-Fc ˜0.18 nM; and 7D VH6-Fc ˜0.23 nM.

FIG. 9 shows inhibition of EGFR phosphorylation in NCI-H1975 cells using Western blot. FIG. 9A: The top panel indicates Western blot lanes corresponding to (1) Medium only; (2) EGF only; (3) 7D VH4-Fc (SEQ ID NO: 8); (4) gp120 (heavy chain Fv SEQ ID NO: 82 and light chain Fv SEQ ID NO: 81); (5) 7D VH6-Fc (SEQ ID NO: 10); (6) EV1 (heavy chain Fv SEQ ID NO: 24 and light chain Fv SEQ ID NO: 26); (7) TV4 (heavy chain Fv SEQ ID NO: 29 and light chain Fv SEQ ID NO: 32); (8) 7D VH4×EV1 bispecific antibody; (9) 7D VH4×TV4 bispecific antibody; (10) 7D VH6×EV1 bispecific antibody; (11) 7D VH6×TV4 bispecific antibody; and (12) cetuximab×gp120 bispecific antibody (monovalent EGFR binding arm). The integrated values are normalized to the b-actin levels in each lane. All 4 candidate BsAbs could inhibit EGFR phosphorylation and had similar inhibition effect as one armed cetuximab×gp120. FIG. 9B: The top panel shows Western blot lanes corresponding to (1) Medium only; (2) EGF only; (3) 7D VH4×EV1; (4) 7D VH4×TV4; (5) 7D VH6×EV1; (6) 7D VH6×TV4; (7) Cetuximab×gp120; (8) gp120; (9) 7D VH4×gp120; (10) 7D VH6×gp120. All of 4 candidate (7D VH4×EV1; 7D VH4×TV4; 7D VH6×EV1; 7D VH6×TV4) BsAbs could inhibit EGFR phosphorylation and had similar inhibition effect with one armed cetuximab×gp120. FIG. 9C shows inhibition of EGFR phosphorylation in H292 and HCC827 cells using Western blot. FIG. 9C: Results for H292 cells with Western blot lanes corresponding to (1) Medium only; (2) EGF only; (3) 7D VH4×EV1; (4) 7D VH4×TV4; (5) 7D VH6×EV1; (6) 7D VH6×TV4; (7) cetuximab×gp120; (8) gp120; (9) 7D VH4×gp120; (10) 7D VH6×gp120. FIG. 9D: Results for HCC827 cells with Western blot lanes corresponding to (1) Medium only; (2) EGF only; (3) 7D VH4×EV1; (4) 7D VH4×TV4; (5) 7D VH6×EV1; (6) 7D VH6×TV4; (7) cetuximab×gp120; (8) gp120; (9) 7D VH4×gp120; (10) 7D VH6×gp120. All of 4 candidate ((7D VH4×EV1; 7D VH4×TV4; 7D VH6×EV1; 7D VH6×TV4)) BsAbs could inhibit EGFR phosphorylation and had the similar inhibition effects with one armed cetuximab×gp120 in H292 cells, but not for HCC827 cells.

FIG. 10 demonstrates TAVO412E utility in a non-small cell lung cancer cell line HCC827 as shown by cell binding, blocking of EGF from binding to EGFR on HCC827 cells, and blocking of HGF from binding to cMET on HCC827 cells. In FIG. 10 A-C, the y axes represented the gMFI values that reflected the binding levels on HCC827 cells, and the x axes represented the concentrations of the test reagents. FIG. 10A shows that TAVO412E had an EC50 value for binding to HCC827 cells of 1.04 nM. The isotype mAb had no binding to HCC827 cells. FIG. 10B shows that TAVO412E had an IC50 value for blocking the binding of EGF to EGFR on HCC827 cells of 2.56 nM. The isotype mAb had no blocking of EGF binding to EGFR on HCC827 cells. FIG. 10C shows that TAVO412E had an IC50 value for blocking the binding of HGF to cMET on HCC827 cells of 0.28 nM. The isotype mAb had no blocking of HGF binding to cMET on HCC827 cells.

FIG. 11 shows inhibition of phosphorylation of EGFR and cMET in H292 and HCC827 cells. In FIG. 11A-B, the y axes were shown as percent values of EGFR phosphorylation as noted in the control mAb and the x axes were concentrations of the test articles. FIG. 11A shows TAVO412E inhibited EGFR phosphorylation in H292 cells in the presence of EGF with an IC50 value of 0.79 nM. The isotype mAb did not inhibit EGFR phosphorylation. FIG. 11B shows TAVO412E inhibited EGFR phosphorylation in H292 cells in the presence of EGF and HGF with an IC50 value of 0.78 nM. The isotype mAb did not inhibit EGFR phosphorylation. In FIG. 11C-D, the y axes are shown as percent cMET phosphorylation as noted in the control mAb and the x axes are concentration of the test articles. FIG. 11C shows TAVO412E inhibited cMET phosphorylation in HCC827 cells in the presence of HGF with an IC50 value of 1.41 nM. The isotype mAb did not inhibit cMET phosphorylation. FIG. 11D shows TAVO412E inhibiting cMET phosphorylation in HCC827 cells in the presence of HGF and EGF with an IC50 value of 1.99 nM. The isotype mAb did not inhibit cMET phosphorylation.

FIG. 12 shows inhibition of proliferation of HCC827 cells. In FIG. 12A-B, the y axes were shown as percent survival rates and the x axes were concentrations of the test articles. FIG. 12A shows TAVO412E inhibited the proliferation of HCC827 cells with an IC50 value of 1.76 nM. The isotype mAb did not inhibit the proliferation of HCC827 cells. FIG. 11B shows TAVO412E inhibited the proliferation of HCC827 cells in the presence of EGF and HGF with an IC50 value of 1.39 nM. The isotype mAb did not inhibit the proliferation of HCC827 cells.

FIG. 13 shows inhibition of cMET phosphorylation in H1975, HCC827, and H292 cells detected by Western blot. FIG. 13A shows results for HCl-H1975; FIG. 13B shows results for HCC827; FIG. 13C shows results for H292 cells. The respective Western blot lanes corresponded to (1) Medium only; (2) HGF only; (3) 7D VH4×EV1; (4) 7D VH4×TV4; (5) 7D VH6×EV1; (6) 7D VH6×TV4; (7) JNJ6372_cMET (cMET heavy chain Fv SEQ ID NO: 77 and cMET light chain Fv SEQ ID NO: 78)×gp120; (8) gp120; (9) EV1×gp120; (10) TV1×gp120. The 4 (7D VH4×EV1; 7D VH4×TV4; 7D VH6×EV1; 7D VH6×TV4) BsAbs had more significant inhibition effect than their monovalent parental Abs in HCC827, H292 and NCI-H1975.

FIG. 14 shows the Fc effector function of TAVO412E on HCC827 cells. In FIG. 14A-B, the y axes are shown as levels of ADCC (FIG. 14A) or ADCP (FIG. 14B) activation and the x axes are concentration of the test articles. In FIG. 14C-E, the y axes are shown as percent lysis and the x axes are concentration of the test articles. FIG. 14A shows TAVO412E induced ADCC reporter activity in the presence of HCC827 cells with an IC50 value of 0.022 nM. The isotype mAb did not induce ADCP reporter activity of HCC827 cells. FIG. 14B shows TAVO412E had ADCP reporter activity on HCC827 cells with an IC50 value of ˜0.27 nM. The isotype mAb did not have ADCP reporter activity of HCC827 cells. FIG. 14C shows TAVO412E had ADCC killing activity on HCC827 cells with an IC50 value of 0.12 nM. The isotype mAb did not have ADCP reporter activity of HCC827 cells. FIG. 14D shows TAVO412E had ADCP killing activity on HCC827 cells with an IC50 value of 0.16 nM. The isotype mAb did not have ADCP reporter activity of HCC827 cells. FIG. 14E shows TAVO412E had CDC killing activity on HCC827 cells with an IC50 value of 3.76 nM. The isotype mAb did not have ADCP reporter activity of HCC827 cells.

FIG. 15 shows anti-tumor activity of TAVO412E on the non-small cell lung cancer cell line NCI-H1975. FIG. 15A shows NCI-H1975 tumor growth inhibition of 42% at 1 mg/kg, 76% at 3 mg/kg, and 94% at 10 mg/kg at day 13. TAVO412E had a dose dependent tumor growth inhibition in H1975 cells. FIG. 15B shows that TAVO412E induced degradation of EGFR in the tumors excised from the in vivo NCI-H1975 xenograft model as well as reduction of EGFR phosphorylation. TAVO412 noted in the western blots referred to TAVO412E. FIG. 15C shows that TAVO412E induced degradation of cMET in the tumors excised from the in vivo NCI-H1975 xenograft model as well as reduction of cMET phosphorylation. TAVO412 noted in the western blots referred to TAVO412E. FIG. 15D shows the bar graph representation of the results for the control isotype mAb and TAVO412E in FIGS. 15B and C. TAVO412E decreased the levels of the total and phosphorylated forms of cMET and EGFR in the tumors excised from in vivo NCI-H1975 xenograft model experiment.

FIG. 16 shows anti-tumor activity of TAVO412E against the NSCLC line HCC827. FIG. 16A shows HCC827 tumor growth inhibition of 45% at 1 mg/kg, 79% at 3 mg/kg, and 94% at 10 mg/kg at day 13. TAVO412E had a dose dependent tumor growth inhibition in HCC827 xenografts. FIG. 16B shows that TAVO412E induced degradation of EGFR and cMET in the tumors excised from the in vivo HCC827 xenograft model experiment. TAVO412 noted in the western blots referred to TAVO412E. FIG. 16C shows the bar graph representation of the quantification results for the control isotype mAb and TAVO412E in FIG. 16B. TAVO412E decreased the levels of the total and phosphorylated forms of cMET and EGFR in the tumors excised from the in vivo HCC827 xenograft model experiment.

FIG. 17 shows anti-tumor activity of TAVO412E against the triple negative breast cancer (TNBC) cell line MDA-MB-468. FIG. 17A shows TAVO412E bound to MDA-MB-468 with an EC50 value for binding of 1.11 nM. TAVO412E had a dose dependent tumor growth inhibition in MDA-MB-468 xenografts. In FIG. 17A, the y axis was gMFI for cell binding and the x axis was concentration of the test article. In FIG. 17B-C, the y axes were percentages of EGFR phosphorylation, and the x axes were the concentrations of the test articles. FIG. 17B shows TAVO412E inhibited human EGFR phosphorylation in MDA-MB-468 cells in the presence of human EGF with an IC50 value of 9.08 nM. The isotype mAb did not inhibit human EGFR phosphorylation. FIG. 17C shows TAVO412E inhibited human EGFR phosphorylation in MDA-MB-468 cells in the presence of human EGF and human HGF with an IC50 value of 8.50 nM. The isotype mAb did not inhibit EGFR phosphorylation.

FIG. 18 shows anti-tumor activity of TAVO412E against the TNBC cell line MDA-MB-231. FIG. 18A shows TAVO412E bound to MDA-MB-231 with an EC50 value for binding of 0.37 nM. In FIG. 18A, the y axis was gMFI for cell binding and the x axis was concentration of the test article. In FIGS. 18B and D, the y axes were luminescence expressed as relative luminescence units (RLU) upon reporter probe activation and the x axes were concentrations of the test articles. FIG. 18B shows TAVO412E had ADCP reporter activity in the presence of MDA-MB-231 cells with an EC50 value of 0.087 nM. The isotype mAb did not have ADCP reporter assay response. In FIGS. 18 C, E, and F, the y axes were percent lysis, and the x axes were concentrations of the test articles. FIG. 18C shows TAVO412E had ADCP killing of MDA-MB-231 cells with an EC50 value of 0.156 nM. The isotype mAb did not have an ADCP killing response. FIG. 18D shows TAVO412E induced ADCC reporter activity in the presence of MDA-MB-231 cells with an EC50 value of 0.13 nM. The isotype mAb did not have ADCP reporter assay response. FIG. 18E shows TAVO412E had ADCC killing of MDA-MB-231 cells with an EC50 value of 0.12 nM. The isotype mAb did not have an ADCC killing response. FIG. 18F shows TAVO412E had CDC killing of MDA-MB-231 cells with an EC50 value of 1.22 nM. The isotype mAb did not have a CDC killing response.

FIG. 19 shows anti-tumor activity of TAVO412E on the triple negative breast cancer cell line MDA-MB-231. FIG. 19A show MDA-MB-231 tumor growth inhibition of 62% at 10 mg/kg dosing at day 20. FIG. 19B shows that TAVO412E induced degradation of EGFR and cMET in the tumors excised from in the in vivo MDA-MB-231 xenograft model experiment. TAVO412 noted in the western blots referred to TAVO412E. FIG. 19C shows the bar graph representation of the quantification results for the control isotype mAb and TAVO412E in FIG. 19B. TAVO412E decreased the levels of the total forms of cMET and EGFR in the in vivo MDA-MB-231 xenograft model experiment.

FIG. 20. Demonstration of TAVO412E utility in gastric cancer cell lines SNU-5 and MKN-45 as shown by cell binding, blocking of HGF from binding to cMET on MKN45 cells, and proliferation inhibition of SNU-5 cells. In FIG. 20A-C, the y axes were gMFI values of cell binding and the x axes were concentrations of the test articles. In FIG. 20D, the y axis was percent survival rate, and the x axis was concentration of the test article. FIG. 20A shows that TAVO412E had an EC50 value for binding to MKN45 cells of 1.78 nM. The isotype mAb had no binding to MKN45 cells. FIG. 20B shows that TAVO412E had an IC50 value for blocking the binding of HGF to cMET on MKN45 cells of 0.28 nM. The isotype mAb had no blocking of HGF binding to cMET on MKN45 cells. FIG. 20C shows that TAVO412E had an EC50 value for binding to SNU-5 cells of 1.99 nM. The isotype mAb did not bind to SNU-5 cells. FIG. 20D shows that TAVO412E had an IC50 value for the inhibition of proliferation of SNU-5 cells of 2.66 nM. The isotype mAb had no inhibition of proliferation of SNU-5 cells.

FIG. 21 shows in vitro anti-tumor activity of TAVO412E on the gastric cancer cell line SNU-5. The experimental protocols were similar to those described in Example 16. In FIG. 21A-B, the y axes were RLU values of ADCC reporter probe assay response and the x axes were concentrations of the test articles. In FIG. 21C, the y axis was percent lysis, and the x axis was concentration of the test article. FIG. 21A shows TAVO412E induced ADCC reporter activity in the presence of SNU-5 cells with an EC50 value of 0.18 nM. The isotype mAb did not have ADCC reporter assay response. FIG. 21B shows TAVO412E had ADCP reporter activity on SNU-5 cells with an EC50 value of 0.20 nM. The isotype mAb did not have ADCP reporter assay response. FIG. 21C shows TAVO412E had CDC killing of SNU-5 cells with an EC50 value of 1.19 nM. The isotype mAb did not have a CDC killing response.

FIG. 22 shows in vivo anti-tumor activity of TAVO412E against the gastric cancer cell line MKN45. FIG. 22A shows MKN-45 tumor growth inhibition of 70% at 3 mg/kg dosing at day 21. FIG. 22B shows that TAVO412E induced degradation of EGFR and cMET in the tumors excised from the in vivo MKN45 xenograft model experiment. TAVO412 noted in the western blots referred to TAVO412E. FIG. 22C shows the bar graph representation of the quantification results for the control isotype mAb and TAVO412E in FIG. 22B. TAVO412E decreased the levels of the total forms of cMET and EGFR in the in vivo MKN45 xenograft model experiment.

FIG. 23. Demonstration of in vitro TAVO412E utility in pancreatic ductal adenocarcinoma cancer cell line BxPC-3 as shown by cell binding, ADCC reporter assay, and ADCP reporter assay. In FIG. 23A, the y axis was gMFI of cell binding and the x axis was concentration of the test article. In FIG. 23B-C, the y axes were RLU of ADCC reporter probe assay response and the x axes were concentrations of the test article. FIG. 23A shows that TAVO412E had an EC50 value for binding to BxPC-3 cells of 0.90 nM. The isotype mAb had no binding to BxPC-3 cells. FIG. 23B shows that TAVO412E had an EC50 value for ADCC reporter assay on BxPC-3 cells of 0.20 nM. The isotype mAb had no ADCC reporter assay activation on BxPC-3 cells. FIG. 23C shows that TAVO412E had an EC50 value for ADCP reporter assay on BxPC-3 cells of 0.65 nM. The isotype mAb had no ADCP reporter assay activation on BxPC-3 cells.

FIG. 24 shows the in vitro inhibition of the phosphorylation of EGFR and cMET in BxPC-3 cells. The experiment was done analogously as what was described in Example 12. In FIG. 24A-D, the y axes were shown as values of percent receptor phosphorylation as noted in the control mAb and the x axes were concentrations of the test articles. FIG. 24A shows TAVO412E inhibited EGFR phosphorylation in BxPC-3 cells in the presence of recombinant human EGF with an IC50 value of 3.45 nM. The isotype mAb did not inhibit EGFR phosphorylation. FIG. 24B shows TAVO412E inhibited cMET phosphorylation in BxPC-3 cells in the presence of recombinant human HGF with an IC50 value of 1.18 nM. The isotype mAb did not inhibit cMET phosphorylation. FIG. 24C shows TAVO412E inhibited EGFR phosphorylation in BxPC-3 cells in the presence of recombinant human EGF and recombinant human HGF with an IC50 value of 1.13 nM. The isotype mAb did not inhibit EGFR phosphorylation. FIG. 24D shows TAVO412E inhibited cMET phosphorylation in BxPC-3 cells in the presence of recombinant human EGF and recombinant human HGF with an IC50 value of 0.44 nM. The isotype mAb did not inhibit cMET phosphorylation.

FIG. 25 shows in vivo anti-tumor activity of TAVO412E on the pancreatic ductal adenocarcinoma cancer cell line BxPC-3. FIG. 25A shows TAVO412E treatment results in BxPC-3 tumor growth inhibition at day 34 of 80% at 10 mg/kg dosing. FIG. 25B shows that TAVO412E induced degradation of EGFR and cMET in the tumors in the in vivo BxPC-3 xenograft model experiment. TAVO412 noted in the western blots referred to TAVO412E. FIG. 25C shows the bar graph representation of the results for the control isotype mAb and TAVO412E in FIG. 25B. TAVO412E decreased the levels of the total forms of cMET and EGFR in the in vivo BxPC-3 xenograft model experiment.

FIG. 26 shows in anti-tumor activity of TAVO412E on the liver cancer cell line HCC9810 in vitro, triple negative breast cancer cell line HCC70 in vivo, and Head and neck cancer cell line FaDu in vivo. FIG. 26A shows TAVO412E had ADCC activity on HCC9810 cell line with an EC50 value of 0.098 nM. In FIG. 26A, the y axis was percent lysis, and the x axis was concentration of the test article. FIG. 26B shows TAVO412E treatment resulted in HCC-70 tumor growth inhibition at day 21 of 26% at 10 mg/kg dosing. FIG. 26C shows TAVO412E treatment resulted in FaDu tumor growth inhibition at day 21 of 95% at 10 mg/kg dosing.

FIG. 27. Demonstration of TAVO412E in vitro anti-tumor activity in head and neck esophageal squamous cell carcinoma cancer cell line KYSE-150 as shown by cell binding, ADCC reporter assay, and ADCC killing assay. In FIG. 27A, the y axis was gMFI for cell binding and the x axis was concentration of the test article. FIG. 27A shows that TAVO412E had an EC50 value for binding to KYSE-150 cells of 0.39 nM. The isotype mAb had no binding to KYSE-150 cells. In FIG. 27B, the y axis was RLU of ADCC reporter probe assay and the x axis was concentration of the test article. FIG. 27B shows that TAVO412E had an EC50 value for ADCC reporter assay on KYSE-150 cells of 0.15 nM. The isotype mAb had no ADCC reporter assay activation on KYSE-150 cells. In FIG. 27C, the y axis was percent lysis, and the x axis was concentration of the test article. FIG. 27C shows that TAVO412E had an EC50 value for ADCC killing response on KYSE-150 cells of 0.038 nM. The isotype mAb had no ADCC killing response on KYSE-150 cells.

FIG. 28. Demonstration of TAVO412E anti-tumor in vitro activity in mesothelioma cancer cell line NCI-H226 as shown by cell binding, ADCC reporter assay, and ADCC killing assay. In FIG. 28A, the y axis was gMFI for cell binding and the x axis was concentration of the test article. FIG. 28A shows that TAVO412E had an EC50 value for binding to NCI-H226 cells of 0.78 nM. The isotype mAb had no binding to NCI-H226 cells. In FIG. 28B, the y axis was RLU of ADCC reporter probe assay and the x axis was concentration of the test article. FIG. 28B shows that TAVO412E had an EC50 value for ADCC reporter assay on NCI-H226 cells of 0.17 nM. The isotype mAb had no ADCC reporter assay activation on NCI-H226 cells. In FIG. 28C, the y axis was percent lysis, and the x axis was concentration of the test article. FIG. 28C shows that TAVO412E had an EC50 value for ADCC killing on NCI-H226 cells of 0.025 nM. The isotype mAb had no ADCC killing response on NCI-H226 cells.

FIG. 29. Demonstration of TAVO412E anti-tumor in vitro activity in colorectal cancer cell line HT-29 as shown by cell binding, ADCC reporter assay, and ADCC killing assay. In FIG. 29A, the y axis was gMFI for cell binding and the x axis was concentration of the test article. FIG. 29A shows that TAVO412E had an EC50 value for binding to HT-29 cells of 0.23 nM. The isotype mAb had no binding to HT-29 cells. In FIG. 29B, the y axis was RLU of ADCC reporter probe assay and the x axis was concentration of the test article. FIG. 28B shows that TAVO412E had an EC50 value for ADCC reporter assay on HT-29 cells of 0.078 nM. The isotype mAb had no ADCC reporter assay activation on HT-29 cells. In FIG. 29C, the y axis was percent lysis, and the x axis was concentration of the test article. FIG. 27C shows that TAVO412E had an EC50 value for ADCC killing response on HT-29 cells of 0.023 nM. The isotype mAb had no ADCC killing response on HT-29 cells.

### DETAILED DESCRIPTION

### Definitions

All publications, including but not limited to patents and patent applications, cited in this specification are herein incorporated by reference as though fully set forth.

It is to be understood that the terminology used herein is for the purpose of describing embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein. In describing and claiming the present disclosure, the following terminology will be used.

As used in this specification and the appended claims, the singular forms “a,” “an,” and “the” include plural referents unless the content clearly dictates otherwise. Thus, for example, reference to “a cell” includes a combination of two or more cells, and the like.

“Antibodies” or “antibody” is meant in a broad sense and includes immunoglobulin molecules including monoclonal antibodies including murine, human, humanized and chimeric monoclonal antibodies, antibody fragments, multispecific or multi-specific antibodies, dimeric, tetrameric, or multimeric antibodies, single chain antibodies, domain antibodies and any other modified configuration of the immunoglobulin molecule that comprises an antigen binding site of the required specificity.

“Full length antibody molecules” are comprised of two heavy chains (HC) and two light chains (LC) inter-connected by disulfide bonds as well as multimers thereof (e.g., IgM). Each heavy chain is comprised of a heavy chain variable region (V_{H}) and a heavy chain constant region (comprised of domains CH1, hinge, CH2 and CH3). Each light chain is comprised of a light chain variable region (V_{L}) and a light chain constant region (C_{L}). The V_{H} and the V_{L} regions may be further subdivided into regions of hyper variability, termed complementarity determining regions (CDR), interspersed with framework regions (FR). Each V_{H} and V_{L} is composed of three CDRs and four FR segments, arranged from amino-to-carboxyl-terminus in the following order: FR1, CDR1, FR2, CDR2, FR3, CDR3 and FR4.

“Complementarity determining regions (CDR)” are “antigen binding sites” in an antibody. CDRs may be defined using various terms: (i) Complementarity Determining Regions (CDRs), three in the V_{H} (HCDR1, HCDR2, HCDR3) and three in the V_{L} (LCDR1, LCDR2, LCDR3) are based on sequence variability (Wu and Kabat 1970) (Kabat et al., Sequences of Proteins of Immunological Interest, 5th Ed. Public Health Service, National Institutes of Health, Bethesda, Md., 1991). (ii) “Hypervariable regions,” “HVR,” or “HV,” three in the V_{H} (H1, H2, H3) and three in the V_{L} (L1, L2, L3) refer to the regions of an antibody variable domains which are hypervariable in structure as defined by Chothia and Lesk (Chothia and Lesk 1987). The International ImMunoGeneTics (IMGT) database (http://www_imgt_org) provides a standardized numbering and definition of antigen-binding sites. The correspondence between CDRs, HVs and IMGT delineations are described (Lefranc, Pommie et al. 2003). The term “CDR,” “HCDR1,” “HCDR2,” “HCDR3,” “LCDR1,” “LCDR2” and “LCDR3” as used herein includes CDRs defined by any of the methods described supra, Kabat, Chothia or IMGT, unless otherwise explicitly stated in the specification.

Immunoglobulins may be assigned to five major classes, IgA, IgD, IgE, IgG and IgM, depending on the heavy chain constant region amino acid sequence. IgA and IgG are further sub-classified as the isotypes IgA₁, IgA₂, IgG₁, IgG₂, IgG₃ and IgG₄. Antibody light chains of any vertebrate species may be assigned to one of two clearly distinct types, namely kappa (κ) and lambda (λ), based on the amino acid sequences of their constant regions.

“Antibody fragment,” “antigen-binding fragment,” or “antigen-binding portion” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (V_{H}), or a light chain variable region (V_{L}). Antibody fragments include well known F_{ab}, F_{(ab′)}2, F_{d} and Fᵥ fragments as well as domain antibodies (dAb) consisting of one V_{H} domain. V_{H} and V_{L} domains may be linked together via a synthetic linker to form various types of single chain antibody designs where the V_{H}/V_{L} domains may pair intramolecularly, or intermolecularly in those cases when the V_{H} and V_{L} domains are expressed by separate single chain antibody constructs, to form a monovalent antigen binding site, such as single chain Fv (scFv) or diabody; described for example in Int. Disclosure Publ. Nos. WO1998/44001, WO1988/01649, WO1994/13804, and WO1992/01047.

“Monoclonal antibody” refers to an antibody population with single amino acid composition in each heavy and each light chain, except for possible well-known alterations such as removal of C-terminal lysine from the antibody heavy chain. Monoclonal antibodies typically bind one antigenic epitope, except that the multispecific monoclonal antibodies bind to multiple such as two distinct antigenic epitopes. Monoclonal antibodies may have heterogeneous glycosylation within the antibody population. Monoclonal antibody may be monospecific or multi-specific, or monovalent, bivalent, or multivalent. A multispecific antibody is included in the term monoclonal antibody.

“Isolated antibody” refers to an antibody or antibody fragment that is substantially free of other antibodies having different antigenic specificities. “Isolated antibody” encompasses antibodies that are isolated to a higher purity, such as antibodies that are 80%, 81%, 82%, 83%, 84%, 85%, 86%, 87%, 88%, 89%, 90%, 91%, 92%, 93%, 94%, 95%, 96%, 97%, 98%, 99% or 100% pure.

“Humanized antibody” refers to an antibody in which the antigen binding sites are derived from non-human species and the variable region frameworks are derived from human immunoglobulin sequences. Humanized antibody may include substitutions in the framework so that the framework may not be an exact copy of expressed human immunoglobulin or human immunoglobulin germLine gene sequences.

“Human antibody” refers to an antibody having heavy and light chain variable regions in which both the framework and the antigen binding site are derived from sequences of human origin and is optimized to have minimal immune response when administered to a human subject. If the antibody contains a constant region or a portion of the constant region, the constant region also is derived from sequences of human origin.

“Anti-target” refers to an antibody or antibody domain that can bind to the specified target molecule such as EGFR (i.e., anti EGFR is an antibody or antibody domain that can bind to EGFR). The style “EGFR” refers to the EGFR protein or EGFR gene product. The style “EGFR” refers to the EGFR gene.

“cMET×EGFR×PD-L1/VEGF” refers to a multispecific antibody or antibody fragments that can bind to cMET, EGFR, and PD-L1 or VEGF. The process of making multispecific antibodies requires the recombinant modifications to parental mAb amino acid sequences. Although the amino acid sequences of the CH1, CL, and Fc domains of each parental mAb will not be the same, there is no significant difference in the binding between the cMET×EGFR and EGFR×cMET multispecific antibodies. The cMET×EGFR×PD-L1/VEGF multispecific can different structural isomers with PD-L1/VEGF that have distinct structure-function activity profiles.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated.

Conventional one and three-letter amino acid codes are used herein as shown in Table 1.

TABLE 1

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The polypeptides, nucleic acids, fusion proteins, and other compositions provided herein may encompass polypeptides, nucleic acids, fusion proteins, and the like that have a recited percent identity to an amino acid sequence or DNA sequence provided herein. The term “identity” refers to a relationship between the sequences of two or more polypeptide molecules or two or more nucleic acid molecules, as determined by aligning and comparing the sequences. “Percent identity,” “percent homology,” “sequence identity,” or “sequence homology” and the like mean the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. In calculating percent identity, the sequences being compared are typically aligned in a way that gives the largest match between the sequences.

The constant region sequences of the mammalian IgG heavy chain are designated in sequence as CH1-hinge-CH2-CH3. The “hinge,” “hinge region” or “hinge domain” of an IgG is generally defined as including Glu216 and terminating at Pro230 of human IgG1 according to the EU Index but functionally, the flexible portion of the chain may be considered to include additional residues termed the upper and lower hinge regions, such as from Glu216 to Gly237 and the lower hinge has been referred to as residues 233 to 239 of the Fc region where FcγR binding was generally attributed. Hinge regions of other IgG isotypes may be aligned with the IgG1 sequence by placing the first and last cysteine residues forming inter-heavy chain S—S bonds. Although boundaries may vary slightly, as numbered according to the EU Index, the CH1 domain is adjacent to the VH domain and amino terminal to the hinge region of an immunoglobulin heavy chain molecule and includes the first (most amino terminal) constant region of an immunoglobulin heavy chain, e.g., from about EU positions 118-215. The Fc domain extends from amino acid 231 to amino acid 447; the CH2 domain is from about Ala231 to Lys340 or Gly341 and the CH3 from about Gly341 or Gln342 to Lys447. The residues of the IgG heavy chain constant region of the CH1 region terminate at Lys. The Fc domain containing molecule comprises at least the CH2 and the CH3 domains of an antibody constant region, and therefore comprises at least a region from about Ala231 to Lys447 of IgG heavy chain constant region. The Fc domain containing molecule may optionally comprise at least a portion of the hinge region.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in proximity in 3-dimensional space through the folding of the protein molecule. Antibody “epitope” depends on the methodology used to identify the epitope.

A “leader sequence” as used herein includes any signal peptide that can be processed by a mammalian cell, including the human B2M leader. Such sequences are well-known in the art.

A “cleavable linker” is a peptide substrate cleavable by an enzyme. Operatively, the cleavable linker, upon being cleaved by the enzyme, allows for activation of the present shielded antibody with a masking domain. Preferably, the cleavable linker is selected so that activation occurs at the desired site of action, which can be a site in or near the target cells (e.g., carcinoma cells) or tissues. For example, the cleavable linker is a peptide substrate specific for an enzyme that is specifically or highly expressed in the site of action, such that the cleavage rate of the cleavable linker in the target site is greater than that in sites other than the target site.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage, and the like.

Furthermore, as used herein, a “polypeptide” refers to a protein that includes modifications, such as deletions, additions, and substitutions (generally conservative in nature as would be known to a person in the art) to the native sequence, if the protein maintains the desired activity. These modifications can be deliberate, as through site-directed mutagenesis, or can be accidental, such as through mutations of hosts that produce the proteins, or errors due to PCR amplification or other recombinant DNA methods.

The term “masking domain” or “shield” or “cap” in this disclosure refers to a protein domain that can be fused to an antibody and mask the antibody in binding to its antigen. The shielding domain can mask the antibody from recognizing its target epitope, so the antibody is kept as an inactive shielded antibody form. Upon the removal of the shielding domain, the variable domains of the antibody are exposed and can bind and exert actions to its target.

The term “recombinant,” as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide sequences with which it is associated in nature. The term “recombinant,” as used with respect to a protein or polypeptide, refers to a polypeptide produced by expression from a recombinant polynucleotide. The term “recombinant,” as used with respect to a host cell or a virus, refers to a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The terms “polynucleotide,” “oligonucleotide,” “nucleic acid” and “nucleic acid molecule” are used interchangeably herein to include a polymeric form of nucleotides, either ribonucleotides or deoxyribonucleotides. This term refers only to the primary structure of the molecule.

“Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems utilizing biological components capable of duplicating a vector. The vector polynucleotide may be DNA or RNA molecules, cDNA, or a hybrid of these, single stranded or double stranded.

“Expression vector” refers to a vector that can be utilized in a biological system or in a reconstituted biological system to direct the translation of a polypeptide encoded by a polynucleotide sequence present in the expression vector.

As used herein, the term “heterologous” used in reference to nucleic acid sequences, proteins, or polypeptides, means that these molecules are not naturally occurring in the cell from which the heterologous nucleic acid sequence, protein or polypeptide was derived. For example, the nucleic acid sequence coding for a human polypeptide that is inserted into a cell that is not a human cell is a heterologous nucleic acid sequence in that context. Whereas heterologous nucleic acids may be derived from different organism or animal species, such nucleic acid need not be derived from separate organism species to be heterologous. For example, in some instances, a synthetic nucleic acid sequence or a polypeptide encoded therefrom may be heterologous to a cell into which it is introduced in that the cell did not previously contain the synthetic nucleic acid. As such, a synthetic nucleic acid sequence or a polypeptide encoded therefrom may be considered heterologous to a human cell, e.g., even if one or more components of the synthetic nucleic acid sequence or a polypeptide encoded therefrom was originally derived from a human cell.

A “host cell,” as used herein, denotes an in vivo or in vitro eukaryotic cell or a cell from a multicellular organism (e.g., a cell line) cultured as a unicellular entity, which eukaryotic cells can be, or have been, used as recipients for a nucleic acid (e.g., an expression vector that comprises a nucleotide sequence encoding a multimeric polypeptide of the present disclosure), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified eukaryotic host cell is genetically modified by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

“Specific binding” or “specifically binds” or “binds” refers to an antibody binding to a specific antigen with greater affinity than for other antigens. Typically, the antibody “specifically binds” when the equilibrium dissociation constant (K_{D}) for binding is about 1×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The K_{D} may be measured using standard procedures.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

A “therapeutically effective amount” or “efficacious amount” refers to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

Before the present disclosure is further described, it is to be understood that this disclosure is not limited to embodiments described, as such may, of course, vary. It is also to be understood that the terminology used herein is for the purpose of describing embodiments only and is not intended to be limiting.

The following examples provide further details which are not intended to limit the scope of disclosure.

### Multispecific Antibody Formats

In some embodiments, the present disclosure provides a multispecific antibody that simultaneously targets two or more of human cMET, EGFR, and PD-L1/VEGF. In some embodiments, the multispecific antibody comprises one or two sets of light chains and zero, one, or two sets of heavy chains. The structure of the light chains and the heavy chains from the respective parental antibodies and various multispecific formats are shown in FIG. 5.

In some embodiments, the present disclosure provides for a combination of shields that can form intermolecular interactions to block Fab arm engagement to their respective epitopes. These intermolecular interactions can involve association of the regions of the heavy chain shield fusion with the regions of the light chain shield fusion.

In some embodiments, the present disclosure provides a multispecific antibody comprising: a human IgG1 heavy chain fusion that comprises from the N- to the C-terminus: signal sequence A-shield A-linker A-protease sequence A-linker B-IgG1 heavy chain; and a human IgG1 light chain fusion that comprises from the N- to the C-terminus, signal sequence B-shield B-linker B-protease sequence B-linker C-IgG1 light chain. In one embodiment, the human IgG1 heavy chain fusion comprises from the N- to the C-terminus: signal sequence A-shield A-linker A-protease sequence A-linker B-IgG1 heavy chain-SD; and the human IgG1 light chain fusion comprises from the N- to the C-terminus, signal sequence B-shield B-linker B-protease sequence B-linker C-IgG1 light chain-SD. SD refers to a single domain that can bind to PD-L1/VEGF (either PD-L1 or VEGF). The shield A can be the same or different from shield B. Linker A can be the same or different from linker B. Protease sequence B can be same or different from protease sequence A.

In some embodiments, the present disclosure provides a multispecific antibody comprising: an anti-EGFR arm (e.g., IgG1 heavy chain and/or IgG1 light chain) comprising a first variable domain that targets EGFR, an anti-cMET arm (e.g., IgG1 heavy chain and/or IgG1 light chain) comprising a second variable domain that targets cMET, and an anti-PD-L1 arm comprising a third variable domain that targets PD-L1 or an anti-VEGF arm comprising a third variable domain that targets VEGF.

In some embodiments, the multispecific antibody comprises a monovalent binding arm that can target EGFR comprising a human IgG1 heavy chain fusion comprising from the N- to the C-terminus, signal sequence A-shield A-linker A-protease sequence A-linker B-IgG1 heavy chain targeting EGFR-anti-PD-L1 or anti-VEGF; and a human IgG1 light chain fusion comprising from the N- to the C-terminus, signal sequence B-shield B-linker B-protease sequence B-linker C-IgG1 light chain targeting EGFR-anti-PD-L1 or anti-VEGF.

In some embodiments, the multispecific antibody comprises a monovalent binding arm that can target cMET comprising a human IgG1 heavy chain fusion comprising from the N- to the C-terminus, signal sequence A-shield A-linker A-protease sequence A-linker B-IgG1 heavy chain targeting cMET-anti-PD-L1 or anti-VEGF; and a human IgG1 light chain fusion comprising amino acid sequences from the N- to the C-terminus, the signal sequence B-shield B-linker B-protease sequence B-linker C-IgG1 light chain targeting cMET-anti-PD-L1 or anti-VEGF.

In some embodiments, the multispecific antibody comprises a monovalent or bivalent binding arm that can target EGFR comprising a human IgG1 heavy chain fusion comprising from the N- to the C-terminus, signal sequence A-shield A-linker A-protease sequence A-linker B-one, two, or more VHO that can bind EGFR-linker C-Fc-anti-PD-L1 or anti-VEGF, wherein the two or more VHOs are optionally connected with one or more linkers or spacers.

In some embodiments, the multispecific antibody comprises a monovalent or bivalent binding arm that can target EGFR comprising a human IgG1 heavy chain fusion comprising from the N- to the C-terminus, signal sequence A, one, two or more VHO that can bind EGFR-linker C-Fc-anti-PD-L1 or anti-VEGF, wherein the two or more VHOs are optionally connected with one or more linkers.

In some embodiments, the multispecific antibody comprises a monovalent binding arm that can target cMET comprising a human IgG1 heavy chain and light chain fusion with one single domain anti-PD-L1 or anti-VEGF.

The present disclosure provides a multispecific antibody that can be generated using well established point mutations in the CH1, CH2, and CH3 domains via controlled Fab arm exchange or via co-expression. In some embodiment, all constructs are symmetric so that there is no preference for the selection of point mutations of the respective parental antibodies.

### Leader Sequences

In certain embodiments, a leader peptide is chosen to drive the secretion of a multispecific antibody described in this disclosure into the cell culture supernatant as a secreted respective parental antibody protein. Any leader peptide for any known secreted proteins/peptides can be used.

As used herein, a “leader peptide” or “signal peptide” includes a short peptide, usually 16-30 amino acids in length, that is present at the N-terminus of most of newly synthesized proteins that are destined towards the secretory pathway. Although lead peptides are extremely heterogeneous in sequence, and many prokaryotic and eukaryotic lead peptides are functionally interchangeable even between different species, the efficiency of protein secretion may be strongly determined by the sequence of the lead/signal peptide.

In certain embodiments, the leader peptide is from a protein residing either inside certain organelles (such as the endoplasmic reticulum, Golgi, or endosomes), secreted from the cell, or inserted into most cellular membranes.

In certain embodiments, the leader peptide is from a eukaryotic protein.

In certain embodiments, the leader peptide is from a secreted protein, e.g., a protein secreted outside a cell.

In certain embodiments, the leader peptide is from a transmembrane protein.

In certain embodiments, the leader peptide contains a stretch of amino acids that is recognized and cleaved by a signal peptidase.

In certain embodiments, the leader peptide does not contain a cleavage recognition sequence of a signal peptidase.

In certain embodiments, the leader peptide is a signal peptide for tissue plasminogen activator (tPA), herpes simplex virus glycoprotein D (HSV gD), a growth hormone, a cytokine, a lipoprotein export signal, CD2, CD3δ, CD3ε, CD3γ, CD3ζ, CD4, CD8α, CD19, CD28, 4-1BB or GM-CSFR, or S. cerevisiae mating factor α-1 signal peptide.

In some embodiments, a leader sequence as described herein may be a mammalian CD4 or CD8 leader sequence, including but not limited to, e.g., a human CD4 or CD8 leader sequence, a non-human primate CD4 or CD8 leader sequence, a rodent CD4 or CD8 leader sequence, and the like. In some embodiments, a CD4 or CD8 leader comprises an amino acid sequence having at least 75%, at least 80%, at least 85%, at least 90%, at least 95%, at least 98%, at least 99%, or 100%, amino acid sequence identity with the human CD4 or CD8 leader sequences.

### Anti-EGFR and Anti-cMET Antibodies, and Multispecific Antibodies

In some embodiments, the disclosure provides for therapeutic cMET, EGFR, and PD-L1/VEGF antibodies and antigen-binding fragments. In some embodiments, shields are attached to the respective Fab domains of the cMET, EGFR, and PD-L1/VEGF antibodies and antigen-binding fragments via protease-cleavage linker sequences to make shielded cMET×EGFR×PD-L1/VEGF multispecific antibodies. The cMET, EGFR, and PD-L1/VEGF targets of such therapeutic antibodies have differential expression levels in pathological sites and normal tissues. The shielded cMET×EGFR×PD-L1/VEGF multispecific antibody remains inactive in normal tissues due to the inhibitory effects of the masking domains on the CDR binding domains. The masking domains are cleaved off by proteases in the disease sites and the shielded cMET×EGFR×PD-L1/VEGF multispecific antibody is converted to the active cMET×EGFR×PD-L1/VEGF multispecific antibody.

In some embodiments, the therapeutic antibodies and fragments applicable for a shielded cMET×EGFR×PD-L1/VEGF multispecific antibody design of the present disclosure encompass full length antibody comprising two heavy chains and two light chains. The antibodies can be human or humanized antibodies. Humanized antibodies include chimeric antibodies and CDR-grafted antibodies. Chimeric antibodies are antibodies that include a non-human antibody variable region linked to a human constant region. CDR-grafted antibodies are antibodies that include the CDRs from a non-human “donor” antibody linked to the framework region from a human “recipient” antibody. Exemplary human or humanized antibodies include IgG, IgM, IgE, IgA, and IgD antibodies. The present antibodies can be of any class (IgG, IgM, IgE, IgA, IgD, etc.) or isotype. For example, a human antibody can comprise an IgG Fc domain, such as at least one of isotypes, IgG1, IgG2, IgG3, or IgG4.

In some embodiments, the present disclosure provides human antibody heavy and light chain sequences that form the CDR binding regions that bind to cMET and EGFR, respectively.

In one aspect, the present disclosure provides an anti-EGFR antibody or an antigen-binding fragment thereof. In some embodiments, the present disclosure provides an anti-EGFR antibody or an antigen-binding fragment thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NOs: 95, 96, and 97;
SEQ ID NOs: 95, 96, and 98;
SEQ ID NOs: 95, 96, and 105;
SEQ ID NOs: 102, 100, and 101; and
SEQ ID NOs: 102, 103, and 104; respectively.

In some embodiments, the disclosure provides an anti-EGFR antibody or antigen binding fragment thereof comprising at least one antibody single domain selected from SEQ ID NOs: 5-12 or antigen binding fragment thereof. In some embodiments, the anti-EGFR antibody or antigen binding fragment thereof comprises tandem antibody single domain heavy chains selected from SEQ ID NOs: 13-18 or antigen binding fragment thereof, wherein two EGFR-binding VHO sequences are linked via a linker.

In some embodiments, the disclosure provides an anti-EGFR antibody or antigen binding fragment thereof comprising at least one antibody single domain having at least 85% (e.g., 85%, 90%, 95%, 96%, 97%, 98%, 99%, or 100%) identity to any one of SEQ ID NOs: 5-12 or antigen binding fragment thereof. In some embodiments, the anti-EGFR antibody or antigen binding fragment thereof comprises tandem antibody single domain heavy chains having at least 85% (e.g., 85%, 90%, 95%, 96%, 97%, 98%, 99%, or 100%) identity to any one of SEQ ID NOs: 13-18 or antigen binding fragment thereof.

In some embodiments, the disclosure provides an anti-EGFR antibody or antigen binding fragment thereof that binds one or more epitopes on human EGFR recognized by an anti-EGFR antibody or antigen binding fragment thereof comprising at least one antibody single domain selected from SEQ ID NOs: 5-12 or comprising tandem antibody single domain heavy chains selected from SEQ ID NOs: 13-18.

In some embodiments, the disclosure provides an anti-EGFR antibody or antigen binding fragment thereof comprising human antibody heavy chain SEQ ID NO: 1 and human antibody light chain SEQ ID NO: 2; or human antibody heavy chain SEQ ID NO: 3 and human antibody light chain SEQ ID NO: 4.

As non-limiting examples, the disclosure provides anti-EGFR heavy and light chain variable region amino acid sequences set forth as SEQ ID NOs: 1-18 with certain CDRs indicated in Table 2.

TABLE 2
Anti-EGFR Antibodies

| | Variable region amino acid | |
|---|---|---|
| Description | sequence (SED ID NO) | CDRs (SEQ ID NOs) |
| Matuzumab | QVQLVQSGAEVKKPGASVKVSCKASG | |
| VH | YTFTSHWMHWVRQAPGQGLEWIGEFN | |
| | PSNGRTNYNEKFKSKATMTVDTSTNT | |
| | AYMELSSLRSEDTAVYYCASRDYDYD | |
| | GRYFDYWGQGTLVTVSS (SEQ ID | |
| | NO: 1) | |
| Matuzumab | DIQMTQSPSSLSASVGDRVTITCSAS | |
| VL | SSVTYMYWYQQKPGKAPKLLIYDTSN | |
| | LASGVPSRFSGSGSGTDYTFTISSLQ | |
| | PEDIATYYCQQWSSHIFTFGQGTKVE | |
| | IK (SEQ ID NO: 2) | |
| Cetuximab | QVQLKQSGPGLVQPSQSLSITCTVSG | |
| VH | FSLTNYGVHWVRQSPGKGLEWLGVIW | |
| | SGGNTDYNTPFTSRLSINKDNSKSQV | |
| | FFKMNSLQSNDTAIYYCARALTYYDY | |
| | EFAYWGQGTLVTVSA (SEQ ID | |
| | NO: 3) | |
| Cetuximab | DILLTQSPVILSVSPGERVSFSCRAS | |
| VL | QSIGTNIHWYQQRTNGSPRLLIKYAS | |
| | ESISGIPSRFSGSGSGTDFTLSINSV | |
| | ESEDIADYYCQQNNNWPTTFGAGTKL | |
| | ELK(SEQ ID NO: 4) | |
| 7D VH1 | EVQLVESGGGVVRPGGSLRLSCAASG | HCDR1: SYGMG (SEQ ID NO: |
| | RTSRSYGMGWFRQAPGKEREFVSGIS | 95) |
| | WRGDSTGYADSVKGRFTISRDNAKNS | HCDR2: GISWRGDSTGYADSVKG |
| | VDLQMNSLRAEDTALYYCAAAAGSAW | (SEQ ID NO: 96) |
| | YGTLYEYDYWGQGTQVTVSS (SEQ | HCDR3: AAGSAWYGTLYEYDY |
| | ID NO: 5) | (SEQ ID NO: 97) |
| 7D VH2 | EVKLEESGGGSVQTGGSLRLTCAASG | HCDR1: SYGMG (SEQ ID NO: |
| | RTSRSYGMGWFRQAPGKEREFVSGIS | 95) |
| | WRGDSTGYADSVKGRFTISRDNAKNT | HCDR2: GISWRGDSTGYADSVKG |
| | VDLQMNSLKPEDTAIYYCAAAAGSAW | (SEQ ID NO: 96) |
| | YGTLYEYDYWGQGTQVTVSS (SEQ | HCDR3: AAGSAWYGTLYEYDY |
| | ID NO: 6) | (SEQ ID NO: 97) |
| 7D VH3 | QVKLEESGGGSVQTGGSLRLTCAASG | HCDR1: SYGMG (SEQ ID NO: |
| | RTSRSYGMGWFRQAPGKEREFVSGIS | 95) |
| | WRGDSTGYADSVKGRFTISRDNAKNT | HCDR2: GISWRGDSTGYADSVKG |
| | VDLQMNSLKPEDTAIYYCAAAAGWAW | (SEQ ID NO: 96) |
| | YGTLYEYDYWGQGTQVTVSS (SEQ | HCDR3: AAGWAWYGTLYEYDY |
| | ID NO: 7) | (SEQ ID NO: 98) |
| 7D VH4 | QVKLEESGGGSVQTGGSLRLTCAASG | HCDR1: SYGMG (SEQ ID NO: |
| | RTSRSYGMGWFRQAPGKEREFVSGIS | 95) |
| | WRGDSTGYADSVKGRFTISRDNAKNT | HCDR2: GISWRGDSTGYADSVKG |
| | VDLQMNSLKPEDTAIYYCAAAAGSAW | (SEQ ID NO: 96) |
| | YGTLYDYDYWGQGTQVTVSS (SEQ | HCDR3: AAGSAWYGTLYDYDY |
| | ID NO: 8) | (SEQ ID NO: 105) |
| 7D VH5 | QVKLEESGGGSVQTGGSLRLTCAASG | HCDR1: SYGMG (SEQ ID NO: |
| | RTSRSYGMGWFRQAPGKEREFVSGIS | 95) |
| | WRGDSTGYADSVKGRFTISRDNAKNT | HCDR2: GISWRGDSTGYADSVKG |
| | VDLQMNSLKPEDTAIYYCAAAAGSAW | (SEQ ID NO: 96) |
| | YGTLYEYDYWGEGTQVTVSS (SEQ | HCDR3: AAGSAWYGTLYEYDY |
| | ID NO: 9) | (SEQ ID NO: 97) |
| 7D VH6 | QVKLEESGGGSVQTGGSLRLTCAASG | HCDR1: SYGMG (SEQ ID NO: |
| | RTSRSYGMGWFRQAPGKEREFVSGIS | 95) |
| | WRGDSTGYADSVKGRFTISRDNAKNT | HCDR2: GISWRGDSTGYADSVKG |
| | VDLQMNSLKPEDTAIYYCAAAAGWAW | (SEQ ID NO: 96) |
| | YGTLYEYDYWGEGTQVTVSS (SEQ | HCDR3: AAGWAWYGTLYEYDY |
| | ID NO: 10) | (SEQ ID NO: 98) |
| EGA1 | QVQLQESGGGLVQPGGSLRLSCAASG | HCDR1: SYAMG (SEQ ID NO: |
| | RTFSSYAMGWFRQAPGKQREFVAAIR | 102) |
| | WSGGYTYYTDSYKGRFTISRDNAKTT | HCDR2: IRWSGGYTYYTDSYKG |
| | VYLQMNSLKPEDTAVYYCAATYLSSD | (SEQ ID NO: 100) |
| | YSRYALPQRPLDYDYWGQGTQVTVSS | HCDR3: TYLSSDYSRYALPQRPL |
| | (SEQ ID NO: 11) | DYDY (SEQ ID NO: 101) |
| 9G8 | EVQLVESGGGLVQAGGSLRLSCAASG | HCDR1: SYAMG (SEQ ID |
| | RTFSSYAMGWFRQAPGKEREFVVAIN | NO: 102) |
| | WSSGSTYYADSVKGRFTISRDNAKNT | HCDR2: AINWSSGSTYYADSVK |
| | MYLQMNSLKPEDTAVYYCAAGYQINS | GRF (SEQ ID NO: 103) |
| | GNYNFKDYEYDYWGQGTQVTVSS | HCDR3: GYQINSGNYNFKDYEYD |
| | (SEQ ID NO: 12) | Y (SEQ ID NO: 104) |
| 7D_VHH6- | QVKLEESGGGSVQTGGSLRLTCAASG | |
| (G4S)2- | RTSRSYGMGWFRQAPGKEREFVSGIS | |
| EGA1 | WRGDSTGYADSVKGRFTISRDNAKNT | |
| | VDLQMNSLKPEDTAIYYCAAAAGWAW | |
| | YGTLYEYDYWGEGTQVTVSSGGGGSG | |
| | GGGSQVQLQESGGGLVQPGGSLRLSC | |
| | AASGRTFSSYAMGWFRQAPGKQREFV | |
| | AAIRWSGGYTYYTDSVKGRFTISRDN | |
| | AKTTVYLQMNSLKPEDTAVYYCAATY | |
| | LSSDYSRYALPQRPLDYDYWGQGTQV | |
| | TVSS (SEQ ID NO: 13) | |
| 7D_VHH6- | QVKLEESGGGSVQTGGSLRLTCAASG | |
| (G4S)2- | RTSRSYGMGWFRQAPGKEREFVSGIS | |
| 9G8 | WRGDSTGYADSVKGRFTISRDNAKNT | |
| | VDLQMNSLKPEDTAIYYCAAAAGWAW | |
| | YGTLYEYDYWGEGTQVTVSSGGGGSG | |
| | GGGSEVQLVESGGGLVQAGGSLRLSC | |
| | AASGRTFSSYAMGWFRQAPGKEREFV | |
| | VAINWSSGSTYYADSVKGRFTISRDN | |
| | AKNTMYLQMNSLKPEDTAVYYCAAGY | |
| | QINSGNYNFKDYEYDYWGQGTQVTVS | |
| | S (SEQ ID NO: 14) | |
| EGA1- | QVQLQESGGGLVQPGGSLRLSCAASG | |
| (G4S)2- | RTFSSYAMGWFRQAPGKQREFVAAIR | |
| 7D_VHH6 | WSGGYTYYTDSVKGRFTISRDNAKTT | |
| | VYLQMNSLKPEDTAVYYCAATYLSSD | |
| | YSRYALPQRPLDYDYWGQGTQVTVSS | |
| | GGGGSGGGGSQVKLEESGGGSVQTGG | |
| | SLRLTCAASGRTSRSYGMGWFRQAPG | |
| | KEREFVSGISWRGDSTGYADSVKGRF | |
| | TISRDNAKNTVDLQMNSLKPEDTAIY | |
| | YCAAAAGWAWYGTLYEYDYWGEGTQV | |
| | TVSS (SEQ ID NO: 15) | |
| 9G8-(G4S) | EVQLVESGGGLVQAGGSLRLSCAASG | |
| 2-7D_VHH6 | RTFSSYAMGWFRQAPGKEREFVVAIN | |
| | WSSGSTYYADSVKGRFTISRDNAKNT | |
| | MYLQMNSLKPEDTAVYYCAAGYQINS | |
| | GNYNFKDYEYDYWGQGTQVTVSSGGG | |
| | GSGGGGSQVKLEESGGGSVQTGGSLR | |
| | LTCAASGRTSRSYGMGWFRQAPGKER | |
| | EFVSGISWRGDSTGYADSVKGRFTIS | |
| | RDNAKNTVDLQMNSLKPEDTAIYYCA | |
| | AAAGWAWYGTLYEYDYWGEGTQVTVS | |
| | S (SEQ ID NO: 16) | |
| 7D_VHH6- | QVKLEESGGGSVQTGGSLRLTCAASG | |
| (G4S)3- | RTSRSYGMGWFRQAPGKEREFVSGIS | |
| BGA1 | WRGDSTGYADSVKGRFTISRDNAKNT | |
| | VDLQMNSLKPEDTAIYYCAAAAGWAW | |
| | YGTLYEYDYWGEGTQVTVSSGGGGSG | |
| | GGGSGGGGSQVQLQESGGGLVQPGGS | |
| | LRLSCAASGRTFSSYAMGWFRQAPGK | |
| | QREFVAAIRWSGGYTYYTDSVKGRFT | |
| | ISRDNAKTTVYLQMNSLKPEDTAVYY | |
| | CAATYLSSDYSRYALPQRPLDYDYWG | |
| | QGTQVTVSS (SEQ ID NO: 17) | |
| 7D_VHH6- | QVKLEESGGGSVQTGGSLRLTCAASG | |
| (G4S)4- | RTSRSYGMGWFRQAPGKEREFVSGIS | |
| EGA1 | WRGDSTGYADSVKGRFTISRDNAKNT | |
| | VDLQMNSLKPEDTAIYYCAAAAGWAW | |
| | YGTLYEYDYWGEGTQVTVSSGGGGSG | |
| | GGGSGGGGSGGGGSQVQLQESGGGLV | |
| | QPGGSLRLSCAASGRTFSSYAMGWFR | |
| | QAPGKQREFVAAIRWSGGYTYYTDSV | |
| | KGRFTISRDNAKTTVYLQMNSLKPED | |
| | TAVYYCAATYLSSDYSRYALPQRPLD | |
| | YDYWGQGTQVTVSS (SEQ ID NO: | |
| | 18) | |

In some embodiments, a multispecific antibody disclosed herein comprises an anti-EGFR antibody or an antigen-binding fragment thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NOs: 95, 96, and 97;
SEQ ID NOs: 95, 96, and 98;
SEQ ID NOs: 95, 96, and 105;
SEQ ID NOs: 102, 100, and 101; and
SEQ ID NOs: 102, 103, and 104; respectively.

In some embodiments, the anti-EGFR arm of a multispecific antibody disclosed herein comprises a human antibody heavy chain SEQ ID NO: 1 and human antibody light chain SEQ ID NO: 2; human antibody heavy chain SEQ ID NO: 3 and human antibody light chain SEQ ID NO: 4. In some embodiments, a multispecific antibody of the disclosure comprises an EGFR binding VHO sequence selected from SEQ ID NOs: 5-12 that is linked to an Fc using a linker selected from SEQ ID NO: 19-22. In some embodiments, a multispecific antibody of the disclosure comprises an EGFR binding VHO sequence selected from SEQ ID NOs: 13-18 that is linked to an Fc using a linker selected from SEQ ID NO: 19-22. The selection of the linker sequence noted in SEQ ID NO: 20 allows for a preferable developability profile.

TABLE 3

| SEQ | | |
|---|---|---|
| ID | | |
| NO: | Description | Amino acid sequence |
| 19 | Linker 1 | GGGGS |
| 20 | Linker 2 | GGGGSGGGGS |
| 21 | Linker 3 | GGGGSGGGGSGGGGS |
| 22 | Linker 4 | GGGGSGGGGSGGGGSGGGGS |

In another aspect, the present disclosure provides an anti-cMET antibody or an antigen-binding fragment thereof. In some embodiments, the present disclosure provides an anti-cMET antibody or an antigen-binding fragment thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NOs: 106, 107, and 133;
SEQ ID NOs: 111, 112, and 113;
SEQ ID NOs: 111, 114, and 113;
SEQ ID NOs: 99, 118, and 119;
SEQ ID NOs: 99, 120, and 119;
SEQ ID NOs: 99, 121, and 119; and
SEQ ID NOs: 99, 122, and 119; respectively;
   and comprising a light chain variable region comprising three CDRs, designated as LCDR1, LCDR2, and LCDR3, wherein the LCDR1, LCDR2, and LCDR3 are selected from:
SEQ ID NOs: 108, 109, and 110;
SEQ ID NOs: 115, 116, and 117; and
SEQ ID NOs: 123, 124, and 125; respectively.

In some embodiments, the present disclosure provides an anti-cMET antibody or an antigen-binding fragment thereof comprising a human antibody heavy chain sequence selected from SEQ ID NOs: 23, 24, 27-29, and 33-37, and a human antibody light chain sequence selected from SEQ ID NOs: 25, 26, 30-32, and 38-40. In some embodiments, the disclosure provides an anti-cMET antibody or an antigen-binding fragment thereof comprising at least one cMET binding VHO sequence selected from SEQ ID NOs: 41-44.

In some embodiments, the present disclosure provides an anti-cMET antibody or an antigen-binding fragment thereof comprising a human antibody heavy chain sequence having at least 85% (e.g., 85%, 90%, 95%, 96%, 97%, 98%, 99%, or 100%) identity to any one of SEQ ID NOs: 23, 24, 27-29, and 33-37, and a human antibody light chain sequence having at least 85% identity to any one of SEQ ID NOs: 25, 26, 30-32, and 38-40. In some embodiments, the disclosure provides an anti-cMET antibody or an antigen-binding fragment thereof comprising at least one cMET binding VHO sequence having at least 85% (e.g., 85%, 90%, 95%, 96%, 97%, 98%, 99%, or 100%) identity to any one of SEQ ID NOs: 41-44.

In some embodiments, the disclosure provides an anti-cMET antibody or antigen binding fragment thereof that binds one or more epitopes on cMET recognized by an anti-cMET antibody or antigen binding fragment thereof comprising an antibody heavy chain sequence selected from SEQ ID NOs: 23, 24, 27-29, and 33-37, and an antibody light chain sequence selected from SEQ ID NOs: 25, 26, 30-32, and 38-40. In some embodiments, the disclosure provides an anti-cMET antibody or antigen binding fragment thereof that binds one or more epitopes on cMET recognized by an anti-cMET antibody or antigen binding fragment thereof comprising at least one cMET binding VHO sequence selected from SEQ ID NOs: 41-44.

In some embodiments, the present disclosure provides an anti-cMET antibody or an antigen-binding fragment thereof comprising a human antibody heavy chain and a human antibody light chain selected from human antibody heavy chain SEQ ID NO: 23 and human antibody light chain SEQ ID NO: 25; human antibody heavy chain SEQ ID NO: 23 and human antibody light chain SEQ ID NO: 26; human antibody heavy chain SEQ ID NO: 24 and human antibody light chain SEQ ID NO: 25; human antibody heavy chain SEQ ID NO: 24 and human antibody light chain SEQ ID NO: 26; human antibody heavy chain SEQ ID NO: 27 and human antibody light chain SEQ ID NO: 30; human antibody heavy chain SEQ ID NO: 27 and human antibody light chain SEQ ID NO: 31; human antibody heavy chain SEQ ID NO: 27 and human antibody light chain SEQ ID NO: 32; human antibody heavy chain SEQ ID NO: 28 and human antibody light chain SEQ ID NO: 30; human antibody heavy chain SEQ ID NO: 28 and human antibody light chain SEQ ID NO: 31; human antibody heavy chain SEQ ID NO: 28 and human antibody light chain SEQ ID NO: 32; human antibody heavy chain SEQ ID NO: 29 and human antibody light chain SEQ ID NO: 30; human antibody heavy chain SEQ ID NO: 29 and human antibody light chain SEQ ID NO: 31; human antibody heavy chain SEQ ID NO: 29 and human antibody light chain SEQ ID NO: 32; human antibody heavy chain SEQ ID NO: 33 and human antibody light chain SEQ ID NO: 38; human antibody heavy chain SEQ ID NO: 33 and human antibody light chain SEQ ID NO: 39; human antibody heavy chain SEQ ID NO: 33 and human antibody light chain SEQ ID NO: 40; human antibody heavy chain SEQ ID NO: 34 and human antibody light chain SEQ ID NO: 38; human antibody heavy chain SEQ ID NO: 34 and human antibody light chain SEQ ID NO: 39; human antibody heavy chain SEQ ID NO: 34 and human antibody light chain SEQ ID NO: 40; human antibody heavy chain SEQ ID NO: 35 and human antibody light chain SEQ ID NO: 38; human antibody heavy chain SEQ ID NO: 35 and human antibody light chain SEQ ID NO: 39; human antibody heavy chain SEQ ID NO: 35 and human antibody light chain SEQ ID NO: 40; human antibody heavy chain SEQ ID NO: 36 and human antibody light chain SEQ ID NO: 38; human antibody heavy chain SEQ ID NO: 36 and human antibody light chain SEQ ID NO: 39; human antibody heavy chain SEQ ID NO: 36 and human antibody light chain SEQ ID NO: 40; human antibody heavy chain SEQ ID NO: 37 and human antibody light chain SEQ ID NO: 38; human antibody heavy chain SEQ ID NO: 37 and human antibody light chain SEQ ID NO: 39; human antibody heavy chain SEQ ID NO: 37 and human antibody light chain SEQ ID NO: 40.

As non-limiting examples, the disclosure provides for anti-cMET heavy and light chain variable region amino acid sequences set forth as SEQ ID NOs: 23-44 and certain CDRs indicated in Table 4.

TABLE 4
Anti-cMET Antibodies

| Description | Amino Acid Sequence (SEQ ID NO) | CDRs (SEQ ID NOs) |
|---|---|---|
| cMET EVH0 | QVQLVQSGAEVKKPGASVKVSCKASG | |
| | YTFTDYYMHWVRQAPGQGLEWMGR | |
| | VNPNRRGTTYNQKFEGRVTMTTDTST | |
| | STAYMELRSLRSDDTAVYYCARANWL | |
| | DYWGQGTTVTVSS (SEQ ID NO: 23) | |
| cMET EVH1 | QVQLVQSGAEVKKPGASVKVSCKASG | HCDR1: DYYMH (SEQ ID NO: 106) |
| | YTFTDYYMHWVRQAPGQGLEWMGR | HCDR2: |
| | VNPNRGGTTYAQKFQGRVTMTTDTS | RVNPNRGGTTYAQKFQG (SEQ |
| | ISTAYMELSRLRSDDTAVYYCARANW | ID NO: 107) |
| | LDYWGQGTTVTVSS (SEQ ID NO: 24) | HCDR3: ANWLDY (SEQ ID NO: |
| | | 133) |
| cMET EVL0 | DIQMTQSPSSLSASVGDRVTITCSVSSS | |
| | VSSIYLHWYQQKPGKAPKLLIYSTSNL | |
| | ASGVPSRFSGSGSGTDFTLTISSLQPED | |
| | FATYYCQVYSGYPLTFGGGTKVEIK | |
| | (SEQ ID NO: 25) | |
| cMET EVL1 | DIQMTQSPSSLSASVGDRVTITCRVSQS | LCDR1: RVSQSVSSIYLH (SEQ ID |
| | VSSIYLHWYQQKPGKAPKLLIYSTSNL | NO: 108) |
| | QSGVPSRFSGSGSGTDFTLTISSLQPED | LCDR2: STSNLQS (SEQ ID NO: |
| | FATYYCQVYSGYPLTFGGGTKVEIK | 109) |
| | (SEQ ID NO: 26) | LCDR3: QVYSGYPLT (SEQ ID |
| | | NO: 110) |
| cMET TVH0 | QVQLVQSGAEVKKPGASVKVSCKASG | |
| | YIFTAYTMHWVRQAPGQGLEWMGWI | |
| | KPNNGLANYAQKFQGRVTMTRDTSIS | |
| | TAYMELSRLRSDDTAVYYCARSEITTE | |
| | FDYWGQGTLVTVSS (SEQ ID NO: 27) | |
| cMET TVH1 | QVQLVQSGAEVKKPGSSVKVSCKASG | HCDR1: AYTMH (SEQ ID NO: 111) |
| | YIFTAYTMHWVRQAPGQGLEWMGGI | HCDR2: |
| | KPNNGLANYAQKFQGRVTITADESTS | GIKPNNGLANYAQKFQG (SEQ |
| | TAYMELSSLRSEDTAVYYCARSEITTE | ID NO: 112) |
| | FDYWGQGTLVTVSS (SEQ ID NO: 28) | HCDR3: SEITTEFDY (SEQ ID NO: |
| | | 113) |
| cMET TVH2 | QVQLQESGPGLVKPSGTLSLTCAVSGY | HCDR1: AYTMH (SEQ ID NO: 111) |
| | IFTAYTMHWVRQPPGKGLEWIGGIKP | HCDR2: GIKPNNGLANYNPSLKS |
| | NNGLANYNPSLKSRATLSVDKSKNQA | (SEQ ID NO: 114) |
| | SMKLSSVTAADTAVYYCARSEITTEF | HCDR3: SEITTEFDY (SEQ ID NO: |
| | DYWGQGTLVTVSS (SEQ ID NO: 29) | 113) |
| cMET TVL0 | DIVMTQSPDSLAVSLGERATINCKSSES | |
| | VDSYANSFLHWYQQKPGQPPKLLIYR | |
| | ASTRESGVPDRFSGSGSGTDFTLTISSL | |
| | QAEDVAVYYCQQSKEDPLTFGGGTKV | |
| | EIK (SEQ ID NO: 30) | |
| cMET TVL1 | DIVMTQTPLSSPVTLGQPASISCRASES | LCDR1: RASESVDSYANSFMH |
| | VDSYANSFMHWLQQRPGQPPRLLIYR | (SEQ ID NO: 115) |
| | ASNLESGVPDRFSGSGAGTDFTLKISR | LCDR2: RASNLES (SEQ ID NO: |
| | VEAEDVGVYYCQQSKEDPLTFGGGT | 116) |
| | KVEIK (SEQ ID NO: 31) | LCDR3: QQSKEDPLT (SEQ ID |
| | | NO: 117) |
| cMET TVL2 | DIQMTQSPSSLSASVGDRVTITCRASES | LCDR1: RASESVDSYANSFMH |
| | VDSYANSFMHWYQQKPGKAPKLLIY | (SEQ ID NO: 115) |
| | RASNLESGVPSRFSGSGSGTDFTLTISS | LCDR2: RASNLES (SEQ ID NO: |
| | LQPEDFATYYCQQSKEDPLTFGGGTK | 116) |
| | VEIK (SEQ ID NO: 32) | LCDR3: QQSKEDPLT (SEQ ID |
| | | NO: 117) |
| cMET OVH0 | EVQLVESGGGLVQPGGSLRLSCAASG | |
| | YTFTSYWLHWVRQAPGKGLEWVGMI | |
| | DPSNSDTRFNPNFKDRFTISADTSKNTA | |
| | YLQMNSLRAEDTAVYYCATYRSYVTP | |
| | LDYWGQGTLVTVSS (SEQ ID NO: 33) | |
| cMET OVH1 | QVQLVQSGAEVKKPGSSVKMSCKASG | HCDR1: SYWLH (SEQ ID NO: 99) |
| | YTFTSYWLHWVRQAPGQGLEWIGMI | HCDR2: MIDPSNSDTRFAQKFQG |
| | DPSNSDTRFAQKFQGRATLTADESTS | (SEQ ID NO: 118) |
| | TAYMELSSLRSEDTAVYYCATYGSYV | HCDR3: YGSYVSPLDY (SEQ ID |
| | SPLDYWGQGTTVTVSS (SEQ ID NO: | NO: 119) |
| | 34) | |
| cMET OVH2 | QVQLQESGPGLVKPSDTLSLTCAASGY | HCDR1: SYWLH (SEQ ID NO: 99) |
| | TFTSYWLHWVRQPPGKGLEWIGMIDP | HCDR2: MIDPSNSDTRENPSLKS |
| | SNSDTRFNPSLKSRVTMSVDTSKNQA | (SEQ ID NO: 120) |
| | SLKLSSVTAVDTAVYYCATYGSYVSP | HCDR3: YGSYVSPLDY (SEQ ID |
| | LDYWGQGTTVTVSS (SEQ ID NO: 35) | NO: 119) |
| cMET OVH3 | EVQLVESGGGLVQPGGSLRLSCAASG | HCDR1: SYWLH (SEQ ID NO: 99) |
| | YTFTSYWLHWVRQAPGKGLEWVGMI | HCDR2: MIDPSNSDTRYAASVKG |
| | DPSNSDTRYAASVKGRFTISVDDSKNS | (SEQ ID NO: 121) |
| | LYLQMNSLKTEDTAVYYCATYGSYVS | HCDR3: YGSYVSPLDY (SEQ ID |
| | PLDYWGQGTTVTVSS (SEQ ID NO: 36) | NO: 119) |
| cMET OVH4 | EVQLVESGGGLVKPGGSVRMSCAASG | HCDR1: SYWLH (SEQ ID NO: 99) |
| | YTFTSYWLHWVRQAPGKGLEWIGMI | HCDR2: MIDPSNSDTRYAAPFKG |
| | DPSNSDTRYAAPFKGRATLSVDDSKN | (SEQ ID NO: 122) |
| | TAYMQLNSLKTEDTAVYYCATYGSY | HCDR3: YGSYVSPLDY (SEQ ID |
| | VSPLDYWGQGTTVTVSS (SEQ ID NO: | NO: 119) |
| | 37) | |
| cMET OVL0 | DIQMTQSPSSLSASVGDRVTITCKSSQS | |
| | LLYTSSQKNYLAWYQQKPGKAPKLLI | |
| | YWASTRESGVPSRFSGSGSGTDFTLTIS | |
| | SLQPEDFATYYCQQYYAYPWTFGQGT | |
| | KVEIK (SEQ ID NO: 38) | |
| cMET OVL1 | DIQMTQSPSTLSASVGDRVTITCRSSQS | LCDR1: RSSQSLLYTSSQKNYLA |
| | LLYTSSQKNYLAWYQQKPGKAPKLLI | (SEQ ID NO: 123) |
| | YWASTRESGVPSRFSGSGSGTEFTLTIS | LCDR2: WASTRES (SEQ ID NO: |
| | SVQPDDFATYYCQQYYAYPWTFGQG | 124) |
| | TKLEIK (SEQ ID NO: 39) | LCDR3: QQYYAYPWT (SEQ ID |
| | | NO: 125) |
| cMET OVL2 | DIVMTQTPPSLPVNPGEPASVSCRSSQS | LCDR1: RSSQSLLYTSSQKNYLA |
| | LLYTSSQKNYLAWYLQKPGQSPQLLI | (SEQ ID NO: 123) |
| | YWASTRESGVPDRFSGSGSGSDFTLKI | LCDR2: WASTRES (SEQ ID NO: |
| | SWVEAEDLGVYYCQQYYAYPWTFGQ | 124) |
| | GTKLEIK (SEQ ID NO: 40) | LCDR3: QQYYAYPWT (SEQ ID |
| | | NO: 125) |
| cMET_4E9v | DVQLVESGGGLVQPGGSLRLSCAASG | |
| VHH | FILDYYAIGWFRQAPGKEREGVLCIDA | |
| | SDDITYYADSVKGRFTISRDNSKNTVY | |
| | LQMNSLRPEDTAVYYCATPIGLSSSCL | |
| | LEYDYDYWGQGTLVTVSS (SEQ ID | |
| | NO: 41) | |
| cMET_4E9z | EVQLVESGGGLVQPGGSLRLSCAASGF | |
| VHH | ILDYYAIGWFRQAPGKEREGVLCIDAS | |
| | DDITYYADSVKGRFTISRDNSKNTVYL | |
| | QMNSLRAEDTAVYYCATPIGLSSSCLL | |
| | EYDYDYWGQGTLVTVSS (SEQ ID NO: | |
| | 42) | |
| cMET_33H10 | EVQLVESGGGLVQAGGSLRLSCAASG | |
| VHH | FTFDDYAIGWFRQAPGEEREGVSSISST | |
| | YGLTYYADSVKGRFTISSSNAKNTVYL | |
| | QMNNLKPEDTAVYYCAATPIERLGLD | |
| | AYEYDYWGQGTQVTVSS (SEQ ID NO: | |
| | 43) | |
| cMET_33H10z | EVQLVESGGGLVQPGGSLRLSCAASGF | |
| VHH | TEDDYAIGWFRQAPGEEREGVSSISST | |
| | YGLTYYADSVKGRFTISSDNSKNTVYL | |
| | QMNSLRAEDTAVYYCAATPIERLGLD | |
| | AYEYDYWGQGTQVTVSS (SEQ ID NO: | |
| | 44) | |

In some embodiments, a multispecific antibody as disclosed herein comprises an anti-cMET antibody or an antigen-binding fragment thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NOs: 106, 107, and 133;
SEQ ID NOs: 111, 112, and 113;
SEQ ID NOs: 111, 114, and 113;
SEQ ID NOs: 99, 118, and 119;
SEQ ID NOs: 99, 120, and 119;
SEQ ID NOs: 99, 121, and 119; and
SEQ ID NOs: 99, 122, and 119; respectively;
   and comprising a light chain variable region comprising three CDRs, designated as LCDR1, LCDR2, and LCDR3, wherein the LCDR1, LCDR2, and LCDR3 are selected from:
SEQ ID NOs: 108, 109, and 110;
SEQ ID NOs: 115, 116, and 117; and
SEQ ID NOs: 123, 124, and 125; respectively.

In some embodiments, the anti-cMET arm of a multispecific antibody as disclosed herein comprises: human antibody heavy chain SEQ ID NO: 23 and human antibody light chain SEQ ID NO: 25; human antibody heavy chain SEQ ID NO: 23 and human antibody light chain SEQ ID NO: 26; human antibody heavy chain SEQ ID NO: 24 and human antibody light chain SEQ ID NO: 25; human antibody heavy chain SEQ ID NO: 24 and human antibody light chain SEQ ID NO: 26; human antibody heavy chain SEQ ID NO: 27 and human antibody light chain SEQ ID NO: 30; human antibody heavy chain SEQ ID NO: 27 and human antibody light chain SEQ ID NO: 31; human antibody heavy chain SEQ ID NO: 27 and human antibody light chain SEQ ID NO: 32; human antibody heavy chain SEQ ID NO: 28 and human antibody light chain SEQ ID NO: 30; human antibody heavy chain SEQ ID NO: 28 and human antibody light chain SEQ ID NO: 31; human antibody heavy chain SEQ ID NO: 28 and human antibody light chain SEQ ID NO: 32; human antibody heavy chain SEQ ID NO: 29 and human antibody light chain SEQ ID NO: 30; human antibody heavy chain SEQ ID NO: 29 and human antibody light chain SEQ ID NO: 31; human antibody heavy chain SEQ ID NO: 29 and human antibody light chain SEQ ID NO: 32; human antibody heavy chain SEQ ID NO: 33 and human antibody light chain SEQ ID NO: 38; human antibody heavy chain SEQ ID NO: 33 and human antibody light chain SEQ ID NO: 39; human antibody heavy chain SEQ ID NO: 33 and human antibody light chain SEQ ID NO: 40; human antibody heavy chain SEQ ID NO: 34 and human antibody light chain SEQ ID NO: 38; human antibody heavy chain SEQ ID NO: 34 and human antibody light chain SEQ ID NO: 39; human antibody heavy chain SEQ ID NO: 34 and human antibody light chain SEQ ID NO: 40; human antibody heavy chain SEQ ID NO: 35 and human antibody light chain SEQ ID NO: 38; human antibody heavy chain SEQ ID NO: 35 and human antibody light chain SEQ ID NO: 39; human antibody heavy chain SEQ ID NO: 35 and human antibody light chain SEQ ID NO: 40; human antibody heavy chain SEQ ID NO: 36 and human antibody light chain SEQ ID NO: 38; human antibody heavy chain SEQ ID NO: 36 and human antibody light chain SEQ ID NO: 39; human antibody heavy chain SEQ ID NO: 36 and human antibody light chain SEQ ID NO:40; human antibody heavy chain SEQ ID NO: 37 and human antibody light chain SEQ ID NO: 38; human antibody heavy chain SEQ ID NO: 37 and human antibody light chain SEQ ID NO: 39; or human antibody heavy chain SEQ ID NO: 37 and human antibody light chain SEQ ID NO: 40, respectively.

In some embodiments, a multispecific antibody of the disclosure comprises an cMET binding VHO sequence selected from SEQ ID NOs: 41-44 that is linked to the Fc using a linker selected from SEQ ID NOs: 19-22.

### Shields

In some embodiments of the disclosure, a therapeutic multispecific cMET x EGFR antibody comprises an anti-cMET antibody arm comprising a masking domain and an anti-EGFR antibody arm comprising a masking domain. Long term administration of anti-cMET or anti-EGFR biologics drugs pose a great risk factor for patients. Thus, the conversion of anti-cMET and/or anti-EGFR antibody arms into a shielded arm with masking domains may increase the safety profile and therapeutic window of the respective antibody arms.

A shield or masking domain is a sequence that can block multispecific antibody CDRs from binding to cMET and EGFR. As non-limiting examples, the disclosure provides for the shield or masking peptide sequence set forth as SEQ ID NO:s 45-51. In some embodiments, SEQ ID NO: 45 is paired with SEQ ID NO: 48; SEQ ID NO: 46 is paired with SEQ ID NO: 48; SEQ ID NO: 47 is paired with SEQ ID NO: 48; SEQ ID NO: 50 is paired with SEQ ID NO: 51; and SEQ ID NO: 49 can pair with itself as either N terminal heavy chain or N terminal light chain fusions.

TABLE 5

| SEQ | | |
|---|---|---|
| ID | | |
| NO: | Description | Amino acid sequence |
| 45 | FI1 | EIDQCIVDDITYNVQDTFHKKHEEGHMLNCTCFGQGRGRWKC |
| | | SPVDQCQDSETGTFYQIGDSWEKYVHGVRYQCYCYGRGIGE |
| | | WHCQPLQTYPSS |
| 46 | FI2 | EIDQCIVDDITYNVQDTFHKKHEEGHMLNCTCFGQGRGRWKC |
| | | EPVDQCQDSETGTFYQIGDSWEKYVHGVRYQCYCYGRGIGE |
| | | WHCQPLQTYPSS |
| 47 | FI3 | EIDQCIVDDITYNVQDTFHKKHEEGHMLQCTCFGQGRGRWKC |
| | | DPVDQCQDSETGTFYQIGDSWEKYVHGVRYQCYCYGRGIGE |
| | | WHCQPLQTYPSS |
| 48 | CO | EVGQRGVVGLPGQRGERGFPGLPGY |
| 49 | IgG1 hinge | EPKSCDKTHTCPPCP |
| 50 | Human IGF2 A chain | GIVEECCFRSCDLALLETYCA |
| 51 | Human IGF2 B chain | AYRPSETLCGGELVDTLQFVCGDRGFYF |

In some embodiments, the present disclosure provides a heavy chain variable region, which can be used as a shielding domain, comprising three Complementarity Determining Regions (CDRs), designated as HCDR1, HCDR2, and HCDR3, wherein the HCDR1, HCDR2, and HCDR3 are selected from:
SEQ ID NOs: 134, 135, and 136;
SEQ ID NOs: 134, 135, and 137;
SEQ ID NOs: 134, 138, and 136;
SEQ ID NOs: 134, 138, and 137;
SEQ ID NOs: 139, 135, and 136;
SEQ ID NOs: 139, 138, and 137; and
SEQ ID NOs: 140, 141, and 136; respectively;

As non-limiting examples, the disclosure provides for shielding domain amino acid sequences set forth as SEQ ID NOs: 52-61 for either the EGFR VHO mAb SEQ ID NOs: 5-12 or 13-18 and the cMET VHO mAb SEQ ID NOs: 41-44 or the cMET mAb SEQ ID NOs: 23-40.

TABLE 6

| Description | Amino acid sequence (SEQ ID NO) | CDRs (SEQ ID NOs) |
|---|---|---|
| K VHO1 | QVQLQESGGGLVQAGGSLRLSCAVSGNT | HCDR1: RYATG (SEQ ID NO: 134) |
| | ISRYATGWFRQTPGNEREFVAAIRWING | HCDR2: AIRWINGNTYYADSVEG (SEQ |
| | NTYYADSVEGRFTISRDSGKNTVYLQMN | ID NO: 135) |
| | NLQPEDTAVYYCASRFLPYASSNAYHEA | HCDR3: RFLPYASSNAYHEALYNYDY |
| | LYNYDYWGQGTQVTVSS (SEQ ID NO: | (SEQ ID NO: 136) |
| | 52) | |
| K VHO2 | QVQLQESGGGLVQAGGSLRLSCAVSGNT | HCDR1: RYATG (SEQ ID NO: 134) |
| | ISRYATGWFRQTPGNEREFVAAIRWING | HCDR2: AIRWTNGNTYYADSVEG (SEQ |
| | NTYYADSVEGRFTISRDSGKNTVYLQMN | ID NO: 135) |
| | NLQPEDTAVYYCASRFLPYASSNAYHES | HCDR3: RFLPYASSNAYHESLYNYDY |
| | LYNYDYWGQGTQVTVSS (SEQ ID NO: | (SEQ ID NO: 137) |
| | 53) | |
| K VHO3 | QVQLQESGGGLVQAGGSLRLSCAVSGNT | HCDR1: RYATG (SEQ ID NO: 134) |
| | ISRYATGWFRQTPGNEREFVAAIRWENG | HCDR2: AIRWENGNTYYADSVEG (SEQ |
| | NTYYADSVEGRFTISRDSGKNTVYLQMN | ID NO: 138) |
| | NLQPEDTAVYYCASRFLPYASSNAYHEA | HCDR3: RFLPYASSNAYHEALYNYDY |
| | LYNYDYWGQGTQVTVSS (SEQ ID NO: | (SEQ ID NO: 136) |
| | 54) | |
| K VHO4 | QVQLQESGGGLVQAGGSLRLSCAVSGNT | HCDR1: RYATG (SEQ ID NO: 134) |
| | ISRYATGWFRQTPGNEREFVAAIRWENG | HCDR2: AIRWENGNTYYADSVEG (SEQ |
| | NTYYADSVEGRFTISRDSGKNTVYLQMN | ID NO: 138) |
| | NLQPEDTAVYYCASRFLPYASSNAYHES | HCDR3: RFLPYASSNAYHESLYNYDY |
| | LYNYDYWGQGTQVTVSS (SEQ ID NO: | (SEQ ID NO: 137) |
| | 55) | |
| CH1 | QVQLQESGGGLVQAGGSLRLSCAVSGNT | HCDR1: GNTISRYATG (SEQ ID NO: 139) |
| VHO1 | ISRYATGWFRQTPGNEREFVAAIRWING | HCDR2: AIRWTNGNTYYADSVEG (SEQ |
| | NTYYADSVEGRFTISRDSGKNTVYLQRF | ID NO: 135) |
| | LPYASSNAYHEALYNYDYWGQGTQVTV | HCDR3: RFLPYASSNAYHEALYNYDY |
| | SS (SEQ ID NO: 56) | (SEQ ID NO: 136) |
| CH1 | QVQLQESGGGLVQAGGSLRLSCAVSGNT | HCDR1: GNTISRYATG (SEQ ID NO: 139) |
| VHO2 | ISRYATGWFRQTPGNEREFVAAIRWENG | HCDR2: AIRWENGNTYYADSVEG (SEQ |
| | NTYYADSVEGRFTISRDSGKNTVYLQRF | ID NO: 138) |
| | LPYASSNAYHESLYNYDYWGQGTQVTV | HCDR3: RFLPYASSNAYHESLYNYDY |
| | SS (SEQ ID NO: 57) | (SEQ ID NO: 137) |
| CH1 | QVQLQESGGGLVQAGGSLRLSCAVSGNT | HCDR1: GNTLSRYAMG (SEQ ID NO: |
| VHO3 | LSRYAMGWFRQAPGNEREFVAAIRWNN | 140) |
| | GNTHYADSVKGRFTISRDSAKNTVYLQ | HCDR2: AIRWNNGNTHYADSVKG |
| | MNNLQPEDTAVYYCASRFLPYASSNAY | (SEQ ID NO: 141) |
| | HEALYNYDYWGQGTQVTVSS (SEQ ID | HCDR3: RFLPYASSNAYHEALYNYDY |
| | NO: 58) | (SEQ ID NO: 136) |
| CH1 | EVQLVESGGGLVQPGGSLRLSCAASGNT | HCDR1: GNTLSRYAMG (SEQ ID NO: |
| VHO4 | LSRYAMGWFRQAPGKEREFVAAIRWNN | 140 |
| | GNTHYVDSVKGRFTISRDNAKNSVYLQ | HCDR2: AIRWNNGNTHYVDSVKG |
| | MNSLRAEDTAVYYCASRFLPYASSNAY | (SEQ ID NO: 141) |
| | HEALYNYDYWGQGTLVTVSS (SEQ ID | HCDR3: RFLPYASSNAYHEALYNYDY |
| | NO: 59) | (SEQ ID NO: 136) |
| CH1 | QVTLKESGPVLVKPTETLTLTCTVSGNTL | HCDR1: GNTLSRYAMG (SEQ ID NO: |
| VHO5 | SRYAMGWFRQAPGKEREFVAAIRWNNG | 140) |
| | NTHYSTSLKSRLTISKDTSKSQVVLTMTN | HCDR2: AIRWNNGNTHYSTSLKS (SEQ |
| | MDPVDTATYYCASRFLPYASSNAYHEA | ID NO: 141) |
| | LYNYDYWGQGTLVTVSS (SEQ ID NO: | HCDR3: RFLPYASSNAYHEALYNYDY |
| | 60) | (SEQ ID NO: 136) |
| CH1 | QVQLVQSGAEVKKPGASVKVSCKASGN | HCDR1: GNTLSRYAMG (SEQ ID NO: |
| VHO6 | TLSRYAMGWFRQAPGQEREFVAAIRWN | 140) |
| | NGNTHYAQKFQGRVTMTRDTSTSTVYM | HCDR2: AIRWNNGNTHYAQKFQG (SEQ |
| | ELSSLRSEDTAVYYCASRFLPYASSNAY | ID NO: 141) |
| | HEALYNYDYWGQGTLVTVSS (SEQ ID | HCDR3: RFLPYASSNAYHEALYNYDY |
| | NO: 61) | (SEQ ID NO: 136) |

As non-limiting examples, the shielding domains can be fused to the N or C terminal ends of the anti-EGFR and anti-cMET binding arms using the linkers listed in SEQ ID NO: 19-22.

### Protease-Cleavable Linker

The protease-cleavable linker, linking the shielding domain to an antibody heavy or light chain, is a peptide substrate cleavable by a protease. The sequence comprises one or more protease substrate sequence and optional linker spacer sequences. The shielding sequences exist as pairs of sequences that can be fused to either the heavy chain or light chain. For each of the two Fab arm domains of the antibody, a shielding sequence is fused to the N-terminus of the antibody heavy chain via one protease-cleavable linker and the complement sequence is fused to the N-terminus of the antibody light chain via another protease-cleavable linker. Alternatively, the linkers can be used to join single domain anti-EGFR, anti-cMET, anti-VEGF, and anti-PD-L1 molecules together.

Many disease tissues, including tumor microenvironment and inflammation site, are abundant with various types of proteases whose overexpression correlate with the disease progression. In disease tissues, the protease-cleavable linker sequences of a shielded antibody are recognized by appropriate type of proteases that releases the shield from the antibody chains. In some embodiment, the protease may cleave two protease-cleavable linkers or one of the two protease-cleavable linker sequences, so the shielding domain is inactive. In either case, the shielding domain would not be able to interfere or block the binding of the Fab arm to its target antigen. As a result, the shielded antibody is converted into active antibody to bind and exert its functional activity to its target.

In some embodiments, the protease-cleavable linker sequences linking the two shielding domains to the two Fab domains in a shielded antibody comprise the same sequences to be cleaved by the same type of protease.

In some embodiments, the protease-cleavable linker sequences linking the two masking domains and the two Fab domains in a shielded antibody comprise different sequences with substrate sequences cleaved by different types of proteases.

Among the family of matrix metalloproteinases (MMPs), MMP2 and MMP9 are up regulated in many types of cancers, including breast, colorectal, pancreatic, gastric, and lung cancers. Besides, the expression and activity of MMP2 and MMP9 also correlates to the progression of many autoimmune disorders and inflammatory diseases, including rheumatoid arthritis, psoriasis, multiple sclerosis, chronic obstructed pulmonary disease, inflammatory bowel disease and osteoporosis (Lin, Lu et al. 2020). The disclosure provides for a protease-cleavable linker sequence comprising a substrate peptide sequence cleaved by MMP2 and MMP9. As non-limiting examples, the disclosure provides for the MMP2, and MMP9 cleavable substrate peptide sequences set forth as SEQ ID NOs: 62-66. As non-limiting examples, the disclosure provides for the MMP3 cleavable substrate peptide sequences set forth as SEQ ID NO: 67.

The urokinase plasminogen activator (uPA) has been reported to be overexpressed in many types of cancer, especially the breast cancer (Banys-Paluchowski, Witzel et al. 2019). uPA is a serine protease that can catalyze the conversion of plasminogen to plasmin which can degrade the basement membrane or extracellular matrix. The matrix degradation can facilitate tumor cells migration and invasion into the surrounding tissue. The disclosure provides for the protease-cleavable linker sequence comprising substrate peptide sequence cleaved by uPA. As non-limiting examples, the disclosure provides for the uPA-cleavable substrate peptide sequence set forth as SEQ ID NOs: 68 and 69.

TABLE 7

| SEQ | | |
|---|---|---|
| ID | | |
| NO: | Description | Amino acid sequence |
| 62 | Substrate sequence for MMP2 and MMP9 | GPLGVR |
| 63 | Substrate sequence for MMP2 and MMP9 | PLGLAR |
| 64 | Substrate sequence for MMP2 and MMP9 | PLGLAG |
| 65 | Substrate sequence for MMP2 and MMP9 | IPVSLRSG |
| 66 | Substrate sequence for MMP2 and MMP9 | GPLGMLSQ |
| 67 | Substrate sequence for MMP3 | RPKPVEVWRK |
| 68 | Substrate sequence for uPA | LSGRSDNH |
| 69 | Substrate sequence for uPA | TGRGPSWV |

The protease-cleavable linker of the present disclosure can include one or more linker peptides interposed between, e.g., shielding sequence and protease substrate peptide sequence, and/or between protease substrate peptide sequence and antibody chains.

### Linkers

Suitable linkers (also referred to as “spacers”) can be readily selected and can be of any of several suitable lengths, such as from 1 amino acid to 30 amino acids (e.g., any specific integer between 1 and 30, or from 1 amino acid (e.g., Gly) to about 20 amino acids, from 2-15, 3-12, 4-10, 5-9, 6-8, or 7-8 amino acids). Exemplary linkers are set forth in Table 3.

Exemplary linkers include glycine polymers (G)ₙ, glycine-serine polymers (including, for example, (GS)ₙ, (GSGGS)ₙ and (GGGS)ₙ, where n is an integer of at least one, e.g., 1, 2, 3, 4, 5, 6, 7, 8, 9, 10, 11, 12, 13, 14, 15, 16, 17, 18, 19, 20), glycine-alanine polymers, alanine-serine polymers, alanine-proline, immunoglobulin isotype and subtype hinge that can comprise IgG₁, IgG₂, IgG₃, IgG₄, IgA, IgE, IgM, and other flexible linkers known in the art. Both Gly and Ser are relatively unstructured, and therefore can serve as a neutral tether between components.

In certain embodiments, the linker is a Glycine polymer. Glycine accesses significantly more phi-psi space than even alanine and is much less restricted than residues with longer side chains (Scheraga 2008). Exemplary linkers can comprise amino acid sequences including, but not limited to: GGS; GGSG; GGSGG; GGGGS; GSGSG; GSGGG; GGGSG; GSSSG, and the like.

In certain embodiments, the linker is an Alanine-Proline polymer. Exemplary linkers can comprise amino acid sequences including, but not limited to (AP)ₙ, where n is an integer of at least one, e.g., 1, 2, 3, 4, 5, 6, 7, 8, 9, 10, 11, 12, 13, 14, 15, 16, 17, 18, 19, 20.

In certain embodiments, the linker is a rigid linker (Chen, Zaro et al. 2013). Exemplary rigid linkers can comprise amino acid sequences including, but not limited to, proline-rich sequence, (XP)ₙ, with X designating any amino acid, preferably Ala, Lys, or Glu, where n is an integer of at least one, e.g., 1, 2, 3, 4, 5, 6, 7, 8, 9, 10, 11, 12, 13, 14, 15, 16, 17, 18, 19, 20. Exemplary rigid linkers can also comprise amino acid sequences including, but not limited to, alpha helix-forming linkers with the sequence of (EAAAK)ₙ, where n is an integer of at least one, e.g., 1, 2, 3, 4, 5, 6, 7, 8, 9, 10, 11, 12, 13, 14, 15, 16, 17, 18, 19, 20.

### Anti-PD-L1 and Anti-VEGF Antibodies

In certain embodiments, an immunomodulatory domain of the present disclosure is a PD-L1 polypeptide. In some cases, a PD-L1 polypeptide of a multimeric polypeptide of the present disclosure comprises an amino acid sequence having at least 75%, at least 80%, at least 85%, at least 90%, at least 95%, at least 98%, at least 99%, or 100%, amino acid sequence identity to amino acids 19-290 of a PD-L1 amino acid sequence as SEQ ID NO: 70.

(SEQ ID NO: 70)
FTVTVPKDLYVVYGSNMTIECKFPVEKQLDLAALIVYWEMEDKNIIQFV
HGEEDLKVQHSSYRQRARLLKDQLSLGNAALQITDVKLQDAGVYRCMIS
YGGADYKRITVKVNAPYNKINQRILVVDPVTSEHELTCQAEGYPKAEVI
WTSSDHQVLSGKTTTTNSKREEKLFNVTSTLRINTTTNEIFYCTFRRLD
PEENHTAELVIPELPLAHPPNERTHLVILGAILLCLGVALTFIFRLRKG
RMMDVKKCGIQDTNSKKQSDTHLEET

In certain embodiments, suitable immunomodulatory domains of the present disclosure include a PD-L1 peptide, the Ig variable domain or scFv format of an anti-PD-L1. In some cases, a single chain Fv polypeptide of anti-PD-L1 antibody of a multimeric polypeptide of the present disclosure comprises an amino acid sequence having at least 75%, at least 80%, at least 85%, at least 90%, at least 95%, at least 98%, at least 99%, or 100%, amino acid sequence identity to a single chain Fv polypeptide of anti-PD-L1 antibody as SEQ ID NO: 71 or SEQ ID NO: 72.

In some embodiments, the present disclosure provides an anti-VEGF antibody or an antigen-binding fragment thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NOs: 129, 130, and 131; and
SEQ ID NOs: 129, 132, and 131; respectively;
   and comprising a light chain variable region comprising three CDRs, designated as LCDR1, LCDR2, and LCDR3, wherein the LCDR1, LCDR2, and LCDR3 are: SEQ ID NOs: 126, 127, and 128, respectively.

In some cases, a single chain Fv polypeptide of anti-VEGF antibody of a multimeric polypeptide of the present disclosure comprises an amino acid sequence having at least 75%, at least 80%, at least 85%, at least 90%, at least 95%, at least 98%, at least 99%, or 100%, amino acid sequence identity to a single chain Fv polypeptide of anti-VEGF antibody as noted in SEQ ID NO: 73, SEQ ID NO: 74, SEQ ID NO: 75, or SEQ ID NO: 76.

In some embodiments, a multispecific antibody as disclosed herein comprises an anti-VEGF antibody or an antigen-binding fragment thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NOs: 129, 130, and 131;
SEQ ID NOs: 129, 132, and 131; respectively;
   and comprising a light chain variable region comprising three CDRs, designated as LCDR1, LCDR2, and LCDR3, wherein the LCDR1, LCDR2, and LCDR3 are: SEQ ID NOs: 126, 127, and 128, respectively.

In some embodiments, the present disclosure provides control bispecific Ab Fv sequences for amivantamab: EGFR binding arms are noted with heavy chain Fv SEQ ID NO: 79 and light chain Fv SEQ ID NO: 80; and cMET binding arms are noted with heavy chain Fv SEQ ID NO: 77 and light chain Fv SEQ ID NO: 78. The null control Ab Fv sequences for the anti-gp120 mAb v12 comprise: heavy chain Fv SEQ ID NO: 82 and light chain as noted in SEQ ID NO: 81.

TABLE 8

| Name | Variable region sequences (SEQ ID NOs) | CDRs (SEQ ID NOs) |
|---|---|---|
| AT PD- | EVQLVESGGGLVQPGGSLRLSCAASGFTFSDSWIHWV | |
| L1 scFv | RQAPGKGLEWVAWISPYGGSTYYADSVKGRFTISADT | |
| | SKNTAYLQMNSLRAEDTAVYYCARRHWPGGFDYWG | |
| | QGTLVTVSSGGGGSGGGGSGGGGSDIQMTQSPSSLSA | |
| | SVGDRVTITCRASQDVSTAVAWYQQKPGKAPKLLIYS | |
| | ASFLYSGVPSRFSGSGSGTDFTLTISSLQPEDFATYYCQ | |
| | QYLYHPATFGQGTKVEIK (SEQ ID NO: 71) | |
| San | EIVLTQSPATLSLSPGERATLSCRASQSVSSYLAWYQQ | |
| PDL1 | KPGQAPRLLIYDASRATGIPARFSGSGSGTDFTLTISS | |
| scFv | LEPEDFAVYYCQQSSNWPRTFGQGTKVEIKGGGGSGG | |
| | GGSGGGGSQVQLVESGGGVVQPGRSLRLDCKASGITF | |
| | SNSGMHWVRQAPGKGLEWVAVIWYDGSKRYYADSV | |
| | KGRFTISRDNSKNTLFLQMNSLRAEDTAVYYCATNDD | |
| | YWGQGTLVTVSS (SEQ ID NO: 72) | |
| VEGF | DIQMTQSPSSLSASVGDRVTITCRASQDISNYLNWYQ | LCDR1: RASQDISNYLN (SEQ |
| VL1- | QKPGKAPKVLIYFTSSLHSGVPSRFSGSGSGTDFTLTIS | ID NO: 126) |
| VH1 | SLQPEDFATYYCQQYSTVPWTFGQGTKLEIKGGGGS | LCDR2: FTSSLHS (SEQ ID NO: |
| scFv | GGGGSGGGGSQVQLVQSGSELKKPGASVKVSCKASG | 127) |
| | YTFTNYGMNWVRQAPGQGLEWMGWINTYTGEPTY | LCDR3: QQYSTVPWT (SEQ ID |
| | AQGFTGRFVFSLDTSVSTAYLQICSLKAEDTAVYFCA | NO: 128) |
| | KYPHYYGSSHWYFDVWGQGTLVTVSS (SEQ ID NO: | HCDR1: NYGMN (SEQ ID NO: |
| | 73) | 129) |
| | | HCDR2: |
| | | WINTYTGEPTYAQGFTG |
| | | (SEQ ID NO: 130) |
| | | HCDR3: YPHYYGSSHWYFDV |
| | | (SEQ ID NO: 131) |
| VEGF | DIQMTQSPSSLSASVGDRVTITCRASQDISNYLNWYQ | LCDR1: RASQDISNYLN (SEQ |
| VL1- | QKPGKAPKVLIYFTSSLHSGVPSRFSGSGSGTDFTLTIS | ID NO: 126) |
| VH2 | SLQPEDFATYYCQQYSTVPWTFGQGTKLEIKGGGGS | LCDR2: FTSSLHS (SEQ ID NO: |
| scFv | GGGGSGGGGSQVQLVQSGAEVKKPGSSVKVSCKASG | 127) |
| | YTFTNYGMNWVRQAPGQGLEWMGWINTYTGEPTY | LCDR3: QQYSTVPWT (SEQ ID |
| | AQKFQGRFTFTADESTSTAYMELSSLRSEDTAVYYCA | NO: 128) |
| | KYPHYYGSSHWYFDVWGQGTLVTVSS (SEQ ID NO: | HCDR1: NYGMN (SEQ ID NO: |
| | 74) | 129) |
| | | HCDR2: |
| | | WINTYTGEPTYAQKFQG |
| | | (SEQ ID NO: 132) |
| | | HCDR3: YPHYYGSSHWYFDV |
| | | (SEQ ID NO: 131) |
| VEGF | QVQLVQSGSELKKPGASVKVSCKASGYTFTNYGMN | HCDR1: NYGMN (SEQ ID NO: |
| VH1- | WVRQAPGQGLEWMGWINTYTGEPTYAQGFTGRFV | 129) |
| VL1 | FSLDTSVSTAYLQICSLKAEDTAVYFCAKYPHYYGSS | HCDR2: |
| scFv | HWYFDVWGQGTLVTVSSGGGGSGGGGSGGGGS | WINTYTGEPTYAQGFTG |
| | DIQMTQSPSSLSASVGDRVTITCRASQDISNYLNWYQ | (SEQ ID NO: 130) |
| | QKPGKAPKVLIYFTSSLHSGVPSRFSGSGSGTDFTLTIS | HCDR3: YPHYYGSSHWYFDV |
| | SLQPEDFATYYCQQYSTVPWTFGQGTKLEIK (SEQ ID | (SEQ ID NO: 131) |
| | NO: 75) | LCDR1: RASQDISNYLN (SEQ |
| | | ID NO: 126) |
| | | LCDR2: FTSSLHS (SEQ ID NO: |
| | | 127) |
| | | LCDR3: QQYSTVPWT (SEQ ID |
| | | NO: 128) |
| VEGF | QVQLVQSGAEVKKPGSSVKVSCKASGYTFTNYGMN | HCDR1: NYGMN (SEQ ID NO: |
| VH2- | WVRQAPGQGLEWMGWINTYTGEPTYAQKFQGRFT | 129) |
| VL1 | FTADESTSTAYMELSSLRSEDTAVYYCAKYPHYYGSS | HCDR2: |
| scFv | HWYFDVWGQGTLVTVSSGGGGSGGGGSGGGGS | WINTYTGEPTYAQKFQG |
| | DIQMTQSPSSLSASVGDRVTITCRASQDISNYLNWYQ | (SEQ ID NO: 132) |
| | QKPGKAPKVLIYFTSSLHSGVPSRFSGSGSGTDFTLTIS | HCDR3: YPHYYGSSHWYFDV |
| | SLQPEDFATYYCQQYSTVPWTFGQGTKLEIK (SEQ ID | (SEQ ID NO: 131) |
| | NO: 76) | LCDR1: RASQDISNYLN (SEQ |
| | | ID NO: 126) |
| | | LCDR2: FTSSLHS (SEQ ID NO: |
| | | 127) |
| | | LCDR3: QQYSTVPWT (SEQ ID |
| | | NO: 128) |
| amivanta | QVQLVQSGAEVKKPGASVKVSCETSGYTFTSYGISWV | |
| mab | RQAPGHGLEWMGWISAYNGYTNYAQKLQGRVTMTT | |
| cMET | DTSTSTAYMELRSLRSDDTAVYYCARDLRGTNYFDY | |
| heavy | WGQGTLVTVSS (SEQ ID NO: 77) | |
| chain Fv | | |
| amivanta | DIQMTQSPSSVSASVGDRVTITCRASQGISNWLAWFQ | |
| mab | HKPGKAPKLLIYAASSLLSGVPSRFSGSGSGTDFTLTIS | |
| cMET | SLQPEDFATYYCQQANSFPITFGQGTRLEIK (SEQ ID | |
| light | NO: 78) | |
| chain Fv | | |
| amivanta | QVQLVESGGGVVQPGRSLRLSCAASGFTFSTYGMHW | |
| mab | VRQAPGKGLEWVAVIWDDGSYKYYGDSVKGRFTISR | |
| EGFR | DNSKNTLYLQMNSLRAEDTAVYYCARDGITMVRGV | |
| heavy | MKDYFDYWGQGTLVTVSS (SEQ ID NO: 79) | |
| chain Fv | | |
| amivanta | AIQLTQSPSSLSASVGDRVTITCRASQDISSALVWYQQ | |
| mab | KPGKAPKLLIYDASSLESGVPSRFSGSESGTDFTLTISSL | |
| EGFR | QPEDFATYYCQQFNSYPLTFGGGTKVEIK (SEQ ID | |
| light | NO: 80) | |
| chain Fv | | |
| gp120 | EIVLTQSPGTLSLSPGERATFSCRSSHSIRSRRVAWYQH | |
| light | KPGQAPRL VIHGVSNRASGISDRFSGSGSGTDFTLTITR | |
| chain Fv | VEPEDFALYYCQVYGASSYTFGQGTKLERK (SEQ ID | |
| | NO: 81) | |
| gp120 | QVQLVQSGAEVKKPGASVKVSCQASGYRFSNFVIHW | |
| heavy | VRQAPGQRFEWMGWINPYNGNKEFSAKFQDRVTFTA | |
| chain Fv | DTSANTAYMELRSLRSADTAVYYCARVGPYSWDDSP | |
| | QDNYYMDVWGKGTTVIVSS (SEQ ID NO: 82) | |

### IgG Fc

In some embodiments, a multispecific antibody such as a shielded cMET x EGFR multispecific antibody may comprise a modified Fc region, wherein the modified Fc region comprises at least one amino acid modification relative to a native Fc region. In some embodiments, a multispecific antibody such as a shielded cMET×EGFR multispecific antibody is provided with a modified Fc region where a naturally occurring Fc region is modified to extend the half-life of the antibody when compared to the parental native antibody in a biological environment, for example, the serum half-life or a half-life measured by an in vitro assay. Exemplary mutations that may be made singularly or in combination are T250Q, M252Y, I253A, S254T, T256E, P257I, T307A, D376V, E380A, M428L, H433K, N434S, N434A, N434H, N434F, H435A and H435R mutations.

In certain embodiments, the extension of half-life can be realized by engineering the M252Y/S254T/T256E mutations in IgG1 Fc residue numbering according to the EU Index (Dall'Acqua, Kiener et al. 2006).

In certain embodiments, the extension of half-life can also be realized by engineering the M428L/N434S mutations in IgG1 Fc (Zalevsky, Chamberlain et al. 2010).

In certain embodiments, the extension of half-life can also be realized by engineering the T250Q/M428L mutations in IgG1 Fc (Hinton, Xiong et al. 2006).

In certain embodiments, the extension of half-life can also be realized by engineering the N434A mutations in IgG1 Fc (Shields, Namenuk et al. 2001).

In certain embodiments, the extension of half-life can also be realized by engineering the T307A/E380A/N434A mutations in IgG1 Fc (Petkova, Akilesh et al. 2006).

The effectiveness of Fc engineering on the extension of antibody half-life can be evaluated in PK studies in mice relative to antibodies with native IgG Fc.

In some embodiments, a shielded cMET×EGFR multispecific antibody is provided with a modified Fc region where a naturally occurring Fc region is modified to enhance the antibody resistance to proteolytic degradation by a protease that cleaves the wild-type antibody between or at residues 222-237 (EU numbering).

In certain embodiments, the resistance to proteolytic degradation can be realized by engineering E233P mutations with G236 deleted in the hinge region when compared to a parental native antibody, residue numbering according to the EU Index (Kinder, Greenplate et al. 2013).

In instances where effector functionality is not desired, the antibodies of the disclosure may further be engineered to introduce at least one mutation in the antibody Fc that reduces binding of the antibody to an activating Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be mutated to reduce binding of an antibody to the activating FcγR and subsequently to reduce effector functions are those described for example in (Xu, Alegre et al. 2000) (Vafa, Gilliland et al. 2014) (Bolt, Routledge et al. 1993, Shields, Namenuk et al. 2001, Chu, Vostiar et al. 2008). Fc mutations with minimal ADCC, ADCP, CDC, and/or Fc mediated cellular activation have been described also as sigma mutations for IgG1, IgG2 and IgG4 (Tam, McCarthy et al. 2017). Exemplary mutations that may be made singularly or in combination are K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S mutations on IgG₁, IgG₂, IgG₃ or IgG₄.

Exemplary combination mutations that may be made to reduce ADCC are L234A/L235A on IgG₁, V234A/G237A/P238S/H268A/V309L/A330S/P331S on IgG₂, F234A/L235A on IgG₄, S228P/F234A/L235A on IgG₄, N297A on IgG₁, IgG₂, IgG₃ or IgG₄, V234A/G237A on IgG₂, K214T/E233P/L234V/L235A/G236 deleted/A327G/P331A/D365E/L358M on IgG₁, H268Q/V309L/A330S/P331S on IgG₂, S267E/L328F on IgG₁, L234F/L235E/D265A on IgG₁, L234A/L235A/G237A/P238S/H268A/A330S/P331S on IgG₁, S228P/F234A/L235A/G237A/P238S on IgG₄, and S228P/F234A/L235A/G236-deleted/G237A/P238S on IgG₄. Hybrid IgG_{2/4} Fc domains may also be used, such as Fc with residues 117-260 from IgG₂ and residues 261-447 from IgG₄.

In some embodiments, a shielded cMET×EGFR multispecific antibody is provided with a modified Fc region where a naturally occurring Fc region is modified to facilitate the generation of multispecific antibody by Fc heterodimerization.

In certain embodiments, the Fc heterodimerization can be realized by engineering F405L and K409R mutations on two parental antibodies and the generation of multispecific antibody in a process known as Fab arm exchange (Labrijn, Meesters et al. 2014).

In certain embodiments, the Fc heterodimerization can also be realized by Fc mutations to facilitate a Knob-in-Hole strategy (see, e.g., Intl. Publ. No. WO 2006/028936). An amino acid with a small side chain (hole) is introduced into one Fc domain and an amino acid with a large side chain (knob) is introduced into the other Fc domain. After co-expression of the two heavy chains, a heterodimer is formed as a result of the preferential interaction of the heavy chain with a “hole” with the heavy chain with a “knob” (Ridgway, Presta et al. 1996). Exemplary Fc mutation pairs forming a knob and a hole are: T366Y/F405A, T366W/F405W, F405W/Y407A, T394W/Y407T, T394S/Y407A, T366W/T394S, F405W/T394S and T366W/T366S/L368A/Y407V. Instead of co-expression, the controlled Fab arm exchange can be applied to generate multispecific antibodies from separate transfections and purification of the corresponding parental antibodies.

In certain embodiments, the Fc heterodimerization can also be realized by Fc mutations to facilitate an electrostatically-matched interactions strategy (Gunasekaran, Pentony et al. 2010). Mutations can be engineered to generate positively charged residues at one Fc domain and negatively charged residues at the other Fc domain as described in US Patent Publ. No. US2010/0015133; US Patent Publ. No. US2009/0182127; US Patent Publ. No. US2010/028637 or US Patent Publ. No. US2011/0123532. Heavy chain heterodimerization can be formed by electrostatically matched interactions between two mutated Fc.

In some embodiments, a shielded cMET×EGFR×PD-L1/VEGF multispecific antibody is provided with a modified Fc region where a naturally occurring Fc region is modified to facilitate the multimerization of the antibody upon interaction with cell surface receptors, although such engineered antibody exists as monomer in solution. The Fc mutations that facilitate antibody multimerization include, but not limited to, E345R mutation, E430G mutation, E345R/E430G mutations, and E345R/E430G/Y440R mutations as described in (Diebolder, Beurskens et al. 2014). Such mutations may also include, but not limited to, T437R mutation, T437R/K248E mutations, and T437R/K338A mutations as described in (Zhang, Armstrong et al. 2017).

Antibodies of the disclosure further comprising conservative modifications are within the scope of the disclosure. “Conservative modifications” refer to amino acid modifications that do not significantly affect or alter the binding characteristics of the antibody containing the amino acid sequences. Conservative modifications include amino acid substitutions, additions, and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis. Amino acid substitutions to the antibodies of the disclosure may be made by known methods for example by PCR mutagenesis (U.S. Disclosure No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be tested for their characteristics using assays described herein.

The antibodies of the disclosure may be post-translationally modified by processes such as glycosylation, isomerization, deglycosylation and/or non-naturally occurring covalent modification such as the addition of polyethylene glycol moieties (pegylation) and lipidation. Such modifications may occur in vivo or in vitro. For example, the antibodies of the disclosure may be conjugated to polyethylene glycol (PEGylated) to improve their pharmacokinetic profiles. Conjugation may be carried out by techniques known to those skilled in the art. Conjugation of therapeutic antibodies with PEG has been shown to enhance pharmacodynamics while not interfering with function.

Antibodies of the disclosure may be modified to improve stability, selectivity, cross-reactivity, affinity, immunogenicity and/or other desirable biological or biophysical property. Stability of an antibody is influenced by a number of factors, including (1) core packing of individual domains that affects their intrinsic stability, (2) protein/protein interface interactions that have impact upon the HC and LC pairing, (3) burial of polar and charged residues, (4) H-bonding network for polar and charged residues; and (5) surface charge and polar residue distribution among other intra- and inter-molecular forces (Worn and Pluckthun 2001). Potential structure destabilizing residues may be identified based upon the crystal structure of an antibody or by molecular modelling in certain cases, and the effect of the residues on antibody stability may be tested by generating and evaluating variants harboring mutations in the identified residues. One of the ways to increase antibody stability is to raise the thermal transition midpoint (Tₘ) as measured by differential scanning calorimetry (DSC). In general, the protein Tₘ is correlated with its stability and inversely correlated with its susceptibility to unfolding and denaturation in solution and the degradation processes that depend on the tendency of the protein to unfold. Several studies have found correlation between the ranking of the physical stability of formulations measured as thermal stability by DSC and physical stability measured by other methods. Formulation studies suggest that a Fab Tₘ has implication for long-term physical stability of a corresponding mAb.

Antibodies of the disclosure may have amino acid substitutions in the Fc region that improve manufacturing and drug stability. An example for IgG1 is H224S (or H224Q) in the hinge 221-DKTHTC-226 (EU numbering) which blocks radically induced cleavage; and for IgG₄, the S228P mutation that blocks half-antibody exchange.

Trispecific cMet×EGFR×VEGF Antibodies

The amino acid sequences of certain components to make a cMet×EGFR x VEGF molecule are exemplified in the table below.

TABLE 9

| SEQ | | |
|---|---|---|
| ID | | |
| NO: | Description | Amino acid sequence |
| 83 | 7D_VHH6-(G4S)2- | QVKLEESGGGSVQTGGSLRLTCAASGRTSRSYGMGWFRQAPGKEREFVSGI |
| | EGA1-Fc_IgG1 | SWRGDSTGYADSVKGRFTISRDNAKNTVDLQMNSLKPEDTAIYYCAAAAG |
| | knob_LPLIL | WAWYGTLYEYDYWGEGTQVTVSSGGGGSGGGGSQVQLQESGGGLVQPG |
| | | GSLRLSCAASGRTFSSYAMGWFRQAPGKQREFVAAIRWSGGYTYYTDSVK |
| | | GRFTISRDNAKTTVYLQMNSLKPEDTAVYYCAATYLSSDYSRYALPQRPLD |
| | | YDYWGQGTQVTVSSGGGGSDKTHTCPPCPAPELLGGPSVFLLPPKPKDTLM |
| | | ISRTPEVTCVVVDVSHEDPEVKFNWYVDGVEVHNAKTKPPEEQYNSTLRV |
| | | VSILTVLHQDWLNGKEYKCKVSNKALPAPIEKTISKAKGQPREPQVYTLPPC |
| | | REEMTKNQVSLWCLVKGFYPSDIAVEWESNGQPENNYKTTPLVLDSDGSFF |
| | | LYSKLTVDKSRWQQGNVFSCSVMHEALHNHYTQKSLSLSPGK |
| 84 | E_VH1- | QVQLVQSGAEVKKPGASVKVSCKASGYTFTDYYMHWVRQAPGQGLEWM |
| | IgG1_hole_LPLIL | GRVNPNRGGTTYAQKFQGRVTMTTDTSISTAYMELSRLRSDDTAVYYCAR |
| | | ANWLDYWGQGTTVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLLPPKPKDTLMISR |
| | | TPEVTCVVVDVSHEDPEVKFNWYVDGVEVHNAKTKPPEEQYNSTLRVVSI |
| | | LTVLHQDWLNGKEYKCKVSNKALPAPIEKTISKAKGQPREPQVCTLPPSRE |
| | | EMTKNQVSLSCAVKGFYPSDIAVEWESNGQPENNYKTTPLVLDSDGSFFLV |
| | | SKLTVDKSRWQQGNVFSCSVMHEALHNHYTQKSLSLSPGK |
| 85 | E_VL1 kappa LC | DIQMTQSPSSLSASVGDRVTITCRVSQSVSSIYLHWYQQKPGKAPKLLIYSTS |
| | | NLQSGVPSRFSGSGSGTDFTLTISSLQPEDFATYYCQVYSGYPLTFGGGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| 86 | 7D_VHH6-(G4S)2- | QVKLEESGGGSVQTGGSLRLTCAASGRTSRSYGMGWFRQAPGKEREFVSGI |
| | EGA1- | SWRGDSTGYADSVKGRFTISRDNAKNTVDLQMNSLKPEDTAIYYCAAAAG |
| | Fc_IgG1_knob_LPLIL- | WAWYGTLYEYDYWGEGTQVTVSSGGGGSGGGGSQVQLQESGGGLVQPG |
| | cleavable linker- | GSLRLSCAASGRTFSSYAMGWFRQAPGKQREFVAAIRWSGGYTYYTDSVK |
| | scFv_VEGF | GRFTISRDNAKTTVYLQMNSLKPEDTAVYYCAATYLSSDYSRYALPQRPLD |
| | | YDYWGQGTQVTVSSGGGGSDKTHTCPPCPAPELLGGPSVFLLPPKPKDTLM |
| | | ISRTPEVTCVVVDVSHEDPEVKFNWYVDGVEVHNAKTKPPEEQYNSTLRV |
| | | VSILTVLHQDWLNGKEYKCKVSNKALPAPIEKTISKAKGQPREPQVYTLPPC |
| | | REEMTKNQVSLWCLVKGFYPSDIAVEWESNGQPENNYKTTPLVLDSDGSFF |
| | | LYSKLTVDKSRWQQGNVFSCSVMHEALHNHYTQKSLSLSPGKGGGGSPLG |
| | | LAGGGGGSDIQMTQSPSSLSASVGDRVTITCRASQDISNYLNWYQQKPGKA |
| | | PKVLIYFTSSLHSGVPSRFSGSGSGTDFTLTISSLQPEDFATYYCQQYSTVPW |
| | | TFGQGTKLEIKGGSEGKSSGSGSESKSTGGSEQVQLVQSGAEVKKPGSSVK |
| | | VSCKASGYTFTNYGMNWVRQAPGQGLEWMGWINTYTGEPTYAQKFQGR |
| | | VTITADESTSTAYMELSSLRSEDTAVYYCAKYPHYYGSSHWYFDVWGQGT |
| | | LVTVSS |
| 87 | E_VH1- | QVQLVQSGAEVKKPGASVKVSCKASGYTFTDYYMHWVRQAPGQGLEWM |
| | IgG1_hole_LPLIL- | GRVNPNRGGTTYAQKFQGRVTMTTDTSISTAYMELSRLRSDDTAVYYCAR |
| | cleavable linker- | ANWLDYWGQGTTVTVSSASTKGPSVFPLAPSSKSTSGGTAALGCLVKDYFP |
| | scFv_VEGF | EPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLLPPKPKDTLMISR |
| | | TPEVTCVVVDVSHEDPEVKFNWYVDGVEVHNAKTKPPEEQYNSTLRVVSI |
| | | LTVLHQDWLNGKEYKCKVSNKALPAPIEKTISKAKGQPREPQVCTLPPSRE |
| | | EMTKNQVSLSCAVKGFYPSDIAVEWESNGQPENNYKTTPLVLDSDGSFFLV |
| | | SKLTVDKSRWQQGNVFSCSVMHEALHNHYTQKSLSLSPGKGGGGSPLGLA |
| | | GGGGGSDIQMTQSPSSLSASVGDRVTITCRASQDISNYLNWYQQKPGKAPK |
| | | VLIYFTSSLHSGVPSRFSGSGSGTDFTLTISSLQPEDFATYYCQQYSTVPWTF |
| | | GQGTKLEIKGGSEGKSSGSGSESKSTGGSEQVQLVQSGAEVKKPGSSVKVS |
| | | CKASGYTFTNYGMNWVRQAPGQGLEWMGWINTYTGEPTYAQKFQGRVTI |
| | | TADESTSTAYMELSSLRSEDTAVYYCAKYPHYYGSSHWYFDVWGQGTLVT |
| | | VSS |
| 88 | 7D_VHH6-(G4S)2- | QVKLEESGGGSVQTGGSLRLTCAASGRTSRSYGMGWFRQAPGKEREFVSGI |
| | EGA1- | SWRGDSTGYADSVKGRFTISRDNAKNTVDLQMNSLKPEDTAIYYCAAAAG |
| | Fc_IgG1_knob_LPLIL- | WAWYGTLYEYDYWGEGTQVTVSSGGGGSGGGGSQVQLQESGGGLVQPG |
| | cleavable linker- | GSLRLSCAASGRTFSSYAMGWFRQAPGKQREFVAAIRWSGGYTYYTDSVK |
| | scFv_VEGF_CC | GRFTISRDNAKTTVYLQMNSLKPEDTAVYYCAATYLSSDYSRYALPQRPLD |
| | | YDYWGQGTQVTVSSGGGGSDKTHTCPPCPAPELLGGPSVFLLPPKPKDTLM |
| | | ISRTPEVTCVVVDVSHEDPEVKFNWYVDGVEVHNAKTKPPEEQYNSTLRV |
| | | VSILTVLHQDWLNGKEYKCKVSNKALPAPIEKTISKAKGQPREPQVYTLPPC |
| | | REEMTKNQVSLWCLVKGFYPSDIAVEWESNGQPENNYKTTPLVLDSDGSFF |
| | | LYSKLTVDKSRWQQGNVFSCSVMHEALHNHYTQKSLSLSPGKGGGGSPLG |
| | | LAGGGGGSDIQMTQSPSSLSASVGDRVTITCRASQDISNYLNWYQQKPGKA |
| | | PKVLIYFTSSLHSGVPSRFSGSGSGTDFTLTISSLQPEDFATYYCQQYSTVPW |
| | | TFGCGTKLEIKGGSEGKSSGSGSESKSTGGSEQVQLVQSGAEVKKPGSSVK |
| | | VSCKASGYTFTNYGMNWVRQAPGQCLEWMGWINTYTGEPTYAQKFQGR |
| | | VTITADESTSTAYMELSSLRSEDTAVYYCAKYPHYYGSSHWYFDVWGQGT |
| | | LVTVSS |
| 89 | E_VH1- | QVQLVQSGAEVKKPGASVKVSCKASGYTFTDYYMHWVRQAPGQGLEWM |
| | IgG1_hole_LPLIL- | GRVNPNRGGTTYAQKFQGRVTMTTDTSISTAYMELSRLRSDDTAVYYCAR |
| | cleavable linker- | ANWLDYWGQGTTVTVSSASTKGPSVFPLAPSSKSTSGGTAALGCLVKDYFP |
| | scFv_VEGF_CC | EPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLLPPKPKDTLMISR |
| | | TPEVTCVVVDVSHEDPEVKFNWYVDGVEVHNAKTKPPEEQYNSTLRVVSI |
| | | LTVLHQDWLNGKEYKCKVSNKALPAPIEKTISKAKGQPREPQVCTLPPSRE |
| | | EMTKNQVSLSCAVKGFYPSDIAVEWESNGQPENNYKTTPLVLDSDGSFFLV |
| | | SKLTVDKSRWQQGNVFSCSVMHEALHNHYTQKSLSLSPGKGGGGSPLGLA |
| | | GGGGGSDIQMTQSPSSLSASVGDRVTITCRASQDISNYLNWYQQKPGKAPK |
| | | VLIYFTSSLHSGVPSRFSGSGSGTDFTLTISSLQPEDFATYYCQQYSTVPWTF |
| | | GCGTKLEIKGGSEGKSSGSGSESKSTGGSEQVQLVQSGAEVKKPGSSVKVS |
| | | CKASGYTFTNYGMNWVRQAPGQCLEWMGWINTYTGEPTYAQKFQGRVTI |
| | | TADESTSTAYMELSSLRSEDTAVYYCAKYPHYYGSSHWYFDVWGQGTLVT |
| | | VSS |
| 90 | 7D_VHH6-(G4S)2- | QVKLEESGGGSVQTGGSLRLTCAASGRTSRSYGMGWFRQAPGKEREFVSGI |
| | EGA1- | SWRGDSTGYADSVKGRFTISRDNAKNTVDLQMNSLKPEDTAIYYCAAAAG |
| | Fc_IgG1_knob_LPLIL- | WAWYGTLYEYDYWGEGTQVTVSSGGGGSGGGGSQVQLQESGGGLVQPG |
| | (G4S)4-scFv_VEGF | GSLRLSCAASGRTFSSYAMGWFRQAPGKQREFVAAIRWSGGYTYYTDSVK |
| | | GRFTISRDNAKTTVYLQMNSLKPEDTAVYYCAATYLSSDYSRYALPQRPLD |
| | | YDYWGQGTQVTVSSGGGGSDKTHTCPPCPAPELLGGPSVFLLPPKPKDTLM |
| | | ISRTPEVTCVVVDVSHEDPEVKFNWYVDGVEVHNAKTKPPEEQYNSTLRV |
| | | VSILTVLHQDWLNGKEYKCKVSNKALPAPIEKTISKAKGQPREPQVYTLPPC |
| | | REEMTKNQVSLWCLVKGFYPSDIAVEWESNGQPENNYKTTPLVLDSDGSFF |
| | | LYSKLTVDKSRWQQGNVFSCSVMHEALHNHYTQKSLSLSPGKGGGGSGG |
| | | GGSGGGGSGGGGSDIQMTQSPSSLSASVGDRVTITCRASQDISNYLNWYQQ |
| | | KPGKAPKVLIYFTSSLHSGVPSRFSGSGSGTDFTLTISSLQPEDFATYYCQQY |
| | | STVPWTFGQGTKLEIKGGSEGKSSGSGSESKSTGGSEQVQLVQSGAEVKKP |
| | | GSSVKVSCKASGYTFTNYGMNWVRQAPGQGLEWMGWINTYTGEPTYAQ |
| | | KFQGRVTITADESTSTAYMELSSLRSEDTAVYYCAKYPHYYGSSHWYFDV |
| | | WGQGTLVTVSS |
| 91 | E_VH1- | QVQLVQSGAEVKKPGASVKVSCKASGYTFTDYYMHWVRQAPGQGLEWM |
| | IgG1_hole_LPLIL- | GRVNPNRGGTTYAQKFQGRVTMTTDTSISTAYMELSRLRSDDTAVYYCAR |
| | (G4S)4-scFv_VEGF | ANWLDYWGQGTTVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLLPPKPKDTLMISR |
| | | TPEVTCVVVDVSHEDPEVKFNWYVDGVEVHNAKTKPPEEQYNSTLRVVSI |
| | | LTVLHQDWLNGKEYKCKVSNKALPAPIEKTISKAKGQPREPQVCTLPPSRE |
| | | EMTKNQVSLSCAVKGFYPSDIAVEWESNGQPENNYKTTPLVLDSDGSFFLV |
| | | SKLTVDKSRWQQGNVFSCSVMHEALHNHYTQKSLSLSPGKGGGGSGGGGS |
| | | GGGGSGGGGSDIQMTQSPSSLSASVGDRVTITCRASQDISNYLNWYQQKPG |
| | | KAPKVLIYFTSSLHSGVPSRFSGSGSGTDFTLTISSLQPEDFATYYCQQYSTV |
| | | PWTFGQGTKLEIKGGSEGKSSGSGSESKSTGGSEQVQLVQSGAEVKKPGSS |
| | | VKVSCKASGYTFTNYGMNWVRQAPGQGLEWMGWINTYTGEPTYAQKFQ |
| | | GRVTITADESTSTAYMELSSLRSEDTAVYYCAKYPHYYGSSHWYFDVWGQ |
| | | GTLVTVSS |
| 92 | 7D_VHH6-(G4S)2- | QVKLEESGGGSVQTGGSLRLTCAASGRTSRSYGMGWFRQAPGKEREFVSGI |
| | EGA1- | SWRGDSTGYADSVKGRFTISRDNAKNTVDLQMNSLKPEDTAIYYCAAAAG |
| | Fc_IgG1_knob_LPLIL- | WAWYGTLYEYDYWGEGTQVTVSSGGGGSGGGGSQVQLQESGGGLVQPG |
| | (G4S)4- | GSLRLSCAASGRTFSSYAMGWFRQAPGKQREFVAAIRWSGGYTYYTDSVK |
| | scFv_VEGF_CC | GRFTISRDNAKTTVYLQMNSLKPEDTAVYYCAATYLSSDYSRYALPQRPLD |
| | | YDYWGQGTQVTVSSGGGGSDKTHTCPPCPAPELLGGPSVFLLPPKPKDTLM |
| | | ISRTPEVTCVVVDVSHEDPEVKFNWYVDGVEVHNAKTKPPEEQYNSTLRV |
| | | VSILTVLHQDWLNGKEYKCKVSNKALPAPIEKTISKAKGQPREPQVYTLPPC |
| | | REEMTKNQVSLWCLVKGFYPSDIAVEWESNGQPENNYKTTPLVLDSDGSFF |
| | | LYSKLTVDKSRWQQGNVFSCSVMHEALHNHYTQKSLSLSPGKGGGGSGG |
| | | GGGSGGGGSGGGGSDIQMTQSPSSLSASVGDRVTITCRASQDISNYLNWYQ |
| | | QKPGKAPKVLIYFTSSLHSGVPSRFSGSGSGTDFTLTISSLQPEDFATYYCQQ |
| | | YSTVPWTFGCGTKLEIKGGSEGKSSGSGSESKSTGGSEQVQLVQSGAEVKK |
| | | PGSSVKVSCKASGYTFTNYGMNWVRQAPGQCLEWMGWINTYTGEPTYAQ |
| | | KFQGRVTITADESTSTAYMELSSLRSEDTAVYYCAKYPHYYGSSHWYFDV |
| | | WGQGTLVTVSS |
| 93 | E_VH1- | QVQLVQSGAEVKKPGASVKVSCKASGYTFTDYYMHWVRQAPGQGLEWM |
| | IgG1_hole_LPLIL- | GRVNPNRGGTTYAQKFQGRVTMTTDTSISTAYMELSRLRSDDTAVYYCAR |
| | (G4S)4- | ANWLDYWGQGTTVTVSSASTKGPSVFPLAPSSKSTSGGTAALGCLVKDYFP |
| | scFv_VEGF_CC | EPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLLPPKPKDTLMISR |
| | | TPEVTCVVVDVSHEDPEVKFNWYVDGVEVHNAKTKPPEEQYNSTLRVVSI |
| | | LTVLHQDWLNGKEYKCKVSNKALPAPIEKTISKAKGQPREPQVCTLPPSRE |
| | | EMTKNQVSLSCAVKGFYPSDIAVEWESNGQPENNYKTTPLVLDSDGSFFLV |
| | | SKLTVDKSRWQQGNVFSCSVMHEALHNHYTQKSLSLSPGKGGGGSGGGGS |
| | | GGGGSGGGGSDIQMTQSPSSLSASVGDRVTITCRASQDISNYLNWYQQKPG |
| | | KAPKVLIYFTSSLHSGVPSRFSGSGSGTDFTLTISSLQPEDFATYYCQQYSTV |
| | | PWTFGCGTKLEIKGGSEGKSSGSGSESKSTGGSEQVQLVQSGAEVKKPGSS |
| | | VKVSCKASGYTFTNYGMNWVRQAPGQCLEWMGWINTYTGEPTYAQKFQ |
| | | GRVTITADESTSTAYMELSSLRSEDTAVYYCAKYPHYYGSSHWYFDVWGQ |
| | | GTLVTVSS |
| 94 | E_VH1- | QVQLVQSGAEVKKPGASVKVSCKASGYTFTDYYMHWVRQAPGQGLEWM |
| | IgG1_hole_LPLIL- | GRVNPNRGGTTYAQKFQGRVTMTTDTSISTAYMELSRLRSDDTAVYYCAR |
| | (G4S)4-scFv_PD-L1 | ANWLDYWGQGTTVTVSSASTKGPSVFPLAPSSKSTSGGTAALGCLVKDYFP |
| | | EPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLLPPKPKDTLMISR |
| | | TPEVTCVVVDVSHEDPEVKFNWYVDGVEVHNAKTKPPEEQYNSTLRVVSI |
| | | LTVLHQDWLNGKEYKCKVSNKALPAPIEKTISKAKGQPREPQVCTLPPSRE |
| | | EMTKNQVSLSCAVKGFYPSDIAVEWESNGQPENNYKTTPLVLDSDGSFFLV |
| | | SKLTVDKSRWQQGNVFSCSVMHEALHNHYTQKSLSLSPGKGGGGSGGGGS |
| | | GGGGSGGGGSDIQMTQSPSSLSASVGDRVTITCRASQDVSTAVAWYQQKP |
| | | GKAPKLLIYSASFLYSGVPSRFSGSGSGTDFTLTISSLQPEDFATYYCQQYLY |
| | | HPATFGCGTKVEIKGGSEGKSSGSGSESKSTGGSEVQLVESGGGLVQPGGSL |
| | | RLSCAASGFTFSDSWIHWVRQAPGKCLEWVAWISPYGGSTYYADSVKGRF |
| | | TISADTSKNTAYLQMNSLRAEDTAVYYCARRHWPGGFDYWGQGTLVTVS |
| | | S |

The amino acid sequence composition of certain multispecific agents are highlighted in the table below. The TAVO412 A to TAVO412H sequences are prepared via co-expression of the open reading frames as noted in the table below.

TABLE 10

| Multispecific Antibody and | Amino acid sequences of complete |
|---|---|
| description | TAVO412 constructs |
| TAVO-412A | SEQ ID NO: 83 + SEQ ID NO: 84 + |
| | SEQ ID NO: 85 |
| TAVO-412B (bivalent scFv with | SEQ ID NO: 86 + SEQ ID NO: 87 + |
| cleavable linker) | SEQ ID NO: 85 |
| TAVO-412C (bivalent scFv with | SEQ ID NO: 90 + SEQ ID NO: 91 + |
| non-cleavable linker) | SEQ ID NO: 85 |
| TAVO-412D (monovalent scFv | SEQ ID NO: 86 + SEQ ID NO: 84 + |
| with cleavable linker) | SEQ ID NO: 85 |
| TAVO-412E (monovalent scFv | SEQ ID NO: 90 + SEQ ID NO: 84 + |
| with non-cleavable linker) | SEQ ID NO: 85 |
| TAVO-412F (monovalent scFv | SEQ ID NO: 83 + SEQ ID NO: 87 + |
| with cleavable linker) | SEQ ID NO: 85 |
| TAVO-412G (monovalent scFv | SEQ ID NO: 83 + SEQ ID NO: 91 + |
| with non-cleavable linker) | SEQ ID NO: 85 |
| TAVO-412H | SEQ ID NO: 83 + SEQ ID NO: 85 + |
| | SEQ ID NO: 94 |

### Expression and Purification of Antibodies

In some embodiments, an antibody such as a multispecific antibody of the present disclosure can be encoded by a single nucleic acid (e.g., a single nucleic acid comprising nucleotide sequences that encode the light and heavy chain polypeptides of the multispecific antibody), or by two or more separate nucleic acids, each of which encode a different part of the parental antibody.

The nucleic acids described herein can be inserted into vectors, e.g., nucleic acid expression vectors and/or targeting vectors. Such vectors can be used in various ways, e.g., for the expression of a shielded antibody with a masking domain described herein in a cell or transgenic animal. Vectors are typically selected to be functional in the host cell in which the vector will be used. A nucleic acid molecule encoding a shielded antibody with a masking domain described herein may be amplified/expressed in prokaryotic, yeast, insect (baculovirus systems) and/or eukaryotic host cells. Selection of the host cell will depend in part on whether the multispecific antibody described herein is to be post-translationally modified (e.g., glycosylated and/or phosphorylated). If so, yeast, insect, or mammalian host cells are preferable. Expression vectors typically contain one or more of the following components: a promoter, one or more enhancer sequences, an origin of replication, a transcriptional termination sequence, a complete intron sequence containing a donor and acceptor splice site, a leader sequence for secretion, a ribosome binding site, a polyadenylation sequence, a polylinker region for inserting the nucleic acid encoding the polypeptide to be expressed, and a selectable marker element.

In most cases, a leader or signal sequence is engineered at the N-terminus of the shielded cMET×EGFR×PD-L1/VEGF multispecific antibody described herein to guide its secretion. The secretion of the shielded cMET×EGFR×PD-L1/VEGF multispecific antibody from a host cell will result in the removal of the signal peptide from the antibody. Thus, the mature the shielded cMET×EGFR×PD-L1/VEGF multispecific antibody will lack any leader or signal sequence. In some cases, such as where glycosylation is desired in a eukaryotic host cell expression system, one may manipulate the various pre-sequences to improve glycosylation or yield. For example, one may alter the peptidase cleavage site of a signal peptide, or add pre-sequences, which also may affect glycosylation.

The disclosure further provides a cell (e.g., an isolated or purified cell) comprising a nucleic acid or vector of the disclosure. The cell can be any type of cell capable of being transformed with the nucleic acid or vector of the disclosure to produce a polypeptide encoded thereby. To express the shielded cMET×EGFR×PD-L1/VEGF multispecific antibody described herein, DNAs encoding partial or full-length light and heavy chains, obtained as described above, are inserted into expression vectors such that the genes are operatively linked to transcriptional and translational control sequences.

Methods of introducing nucleic acids and vectors into isolated cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation.

After introducing the nucleic acid or vector of the disclosure into a cell, the cell is cultured under conditions suitable for expression of the encoded sequence. The antibody, antigen binding fragment, or portion of the antibody then can be isolated from the cell.

In certain embodiments, two or more vectors that together encode a shielded cMET×EGFR×PD-L1/VEGF multispecific antibody described herein, can be introduced into the cell.

In certain embodiments, purification of a shielded cMET×EGFR×PD-L1/VEGF multispecific antibody described herein which has been secreted into the cell media, can be accomplished using a variety of techniques including affinity, immunoaffinity or ion exchange chromatography, molecular sieve chromatography, preparative gel electrophoresis or isoelectric focusing, chromatofocusing, and high-pressure liquid chromatography. For example, antibodies comprising a Fc region may be purified by affinity chromatography with Protein A, which selectively binds the Fc region.

Modified forms of a shielded cMET×EGFR×PD-L1/VEGF multispecific antibody may be prepared with affinity tags, such as hexahistidine or other small peptide such as FLAG (Eastman Kodak Co., New Haven, Conn.) or Myc (Invitrogen) at either its carboxyl or amino terminus and purified by a one-step affinity column. For example, Poly histidine binds with great affinity and specificity to nickel, thus an affinity column of nickel (such as the Qiagen® nickel columns) can be used for purification of Poly histidine-tagged selective binding agents. In some instances, more than one purification step may be employed.

### Effects of Multispecific Antibodies on Binding and Functional Activity

In some embodiments, masking domains on a shielded cMET×EGFR×PD-L1/VEGF multispecific antibody can inhibit or block the capability of the Fab arms to bind to the respective antigens, cMET, EGFR, and PD-L1/VEGF. The masking domains may reduce the maximum binding capacity of the shielded multispecific antibody in binding to the respective antigens. The masking domains may also reduce the binding affinity of the shielded multispecific antibody in binding to the respective antigens.

When the masking domains are cleaved off by proteases, the shielded antibody is converted to an active multispecific antibody with the restoration of the capability of the antibody in binding to its targets. The removal of masking domains from the shielded multispecific antibody can be realized by in vitro protease cutting assay using recombinant or purified protease. The removal of the masking domains from the shielded multispecific antibody can also be realized in vivo by proteases overexpressed in disease site. The removal of the masking domains can be assessed by comparing the molecular weight of heavy chain and light chain of shielded antibodies with the masking domain to the active antibody without the masking domain by SDS-PAGE, IEX, or HIC analyses.

In vitro and cell-based assays are well described in the art for use in determining a shielded antibody, active antibody, and converted antibody after protease cleavage in binding to its antigen. For example, the binding of an antibody may be determined by ELISA by immobilizing a recombinant or purified antigen, sequestering the antibody with the immobilized antigen and determining the amount of bound antibody. This can also be performed using a Biacore® instrument for kinetic analysis of binding interactions. For cell-based binding assay, the binding of an antibody may be determined by flow cytometry by incubating the antibody with cells expressing antigens on cell surface and determining the amount of antibody bound to cell surface antigen.

### Pharmaceutical Compositions

In some embodiments, an antibody of the disclosure, e.g., a shielded cMET x EGFR×PD-L1/VEGF multispecific antibody, for use according to the present disclosure can be formulated in compositions, especially pharmaceutical compositions, for use in the methods herein. Such a composition comprises a therapeutically or prophylactically effective amount of a multispecific antibody described in this disclosure in mixture with a suitable carrier, e.g., a pharmaceutically acceptable agent. Typically, the multispecific antibody described in this disclosure are sufficiently purified for administration to an animal before formulation in a pharmaceutical composition.

Pharmaceutically acceptable agents include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

The composition can be in a liquid form or in a lyophilized or freeze-dried form and may include one or more lyoprotectants, excipients, surfactants, high molecular weight structural additives and/or bulking agents.

Compositions can be suitable for parenteral administration. Exemplary compositions are suitable for injection or infusion into an animal by any route available to a skilled person, such as intraarticular, subcutaneous, intravenous, intramuscular, intraperitoneal, intracerebral (intraparenchymal), intracerebroventricular, intramuscular, intraocular, intraarterial, intralesional, intrarectal, transdermal, oral, and inhaled routes.

Pharmaceutical compositions described herein can be formulated for controlled or sustained delivery in a manner that provides local concentration of the product (e.g., bolus, depot effect) sustained release and/or increased stability or half-life in a particular local environment.

### Methods of Use

In some embodiments, an antibody of the disclosure, e.g., a shielded cMET x EGFR×PD-L1/VEGF multispecific antibody described herein, is useful for the treatment of gastric, lung, pancreatic, colorectal, and/or other cancers. In contrast to corresponding therapeutic antibodies, a shielded cMET×EGFR×PD-L1/VEGF multispecific antibody may have comparable efficacy in treating these diseases due to the conversion of the shielded antibody to an active antibody specifically in disease sites by the removal of the shielding domain by proteases overexpressed in disease sites. However, the shielded antibody may have reduced systematic toxicity due to the masking of the antibody activity by the shielding domain in normal tissues that lack enough proteases needed to cleave off the masking domain. In short, the shielded multispecific antibody described herein may be efficacious as the corresponding therapeutic antibody in treating diseases but with much improved safety profile. Due to the improved safety profile, increased levels of dosing comprising the shielded multispecific antibodies may be administered to the patient with improved treatment efficacy.

In some embodiments, the disclosure provides for a method of treating cancer in a subject, comprising administering to the subject a therapeutically effective amount of a shielded cMET×EGFR×PD-L1/VEGF multispecific antibody. The disclosure also provides for use of a shielded multispecific provided herein in a method of treating cancer; and for use of a shielded cMET×EGFR×PD-L1/VEGF multispecific antibodies provided herein in the manufacture of a medicament for use in cancer. Exemplary cancers include but are not limited to non-small cell lung cancer, female breast cancer, pancreatic cancer, colorectal cancer, and peritoneum cancer.

All combinations of the various elements described herein are within the scope of the disclosure unless otherwise indicated herein or otherwise clearly contradicted by context.

This disclosure will be better understood from the following Experimental Details. However, one skilled in the art will readily appreciate that the specific methods and results discussed are merely illustrative of the disclosure.

### Example 1: Expression and Purification of the Anti-EGFR and Anti-cMET Antibodies

An anti-cMET antibody and an anti-EGFR antibody were generated. They were employed to evaluate a multispecific antibody. Heavy chain and light chain constructs expressing anti-EGFR, anti-cMET, anti-cMET×anti-VEGF and anti-EGFR×anti-VEGF parental mAbs were prepared. Plasmids encoding heavy chains and light chains of these anti-cMET, anti-VEGF, and anti-EGFR antibodies were co-transfected into Expi293F cells following the transfection kit instructions (Thermo Scientific). Cells were spun down on day 5 post transfection, and the supernatant were passed through a 0.2 μm filter. The purification of the expressed antibodies from the supernatants were achieved by affinity chromatography over protein A agarose columns (GE Healthcare Life Sciences). The purified antibodies were buffer exchanged into DPBS, pH 7.2 by dialysis, and protein concentrations were determined by UV absorbance at 280 nm. The cMet×EGFR×VEGF Abs in a human IgG₁ backbone with knob in hole mutations were expressed in Chinese hamster ovary (CHO) cell line, purified by standard Protein A affinity capture followed by iron exchange chromatography. The proteins were monomeric in SEC and pure via SDS-PAGE.

### Example 2: Expression and Purification of Shielded Anti-EGFR and Anti-cMET Parental Antibodies

An anti-cMET and an anti-EGFR antibody were employed to evaluate a shielded multispecific antibody. Heavy chain and light chain constructs expressing shielded anti-cMET and anti-EGFR parental mAbs were prepared. Plasmids encoding heavy chains and light chains of these shielded anti-cMET, and anti-EGFR antibodies were co-transfected into Expi293F cells following the transfection kit instructions (Thermo Scientific). Cells were spun down on day 5 post transfection, and the supernatants were passed through a 0.2 μm filter. The purification of expressed antibodies from the supernatants were achieved by affinity chromatography over protein A agarose columns (GE Healthcare Life Sciences). The purified antibodies were buffer exchanged into DPBS, pH 7.2 by dialysis, and protein concentrations were determined by UV absorbance at 280 nm.

Example 3: Digestion of Shielded Antibody Comprising Masking Domain with Proteases

In vitro protease cutting assays are set up to evaluate whether the shielding domain can be removed from masked Abs by proteases. For MMP2, recombinant human MMP2 is activated by incubating with p-amino phenylmercuric acetate (APMA) according to manufacturer's instruction (R&D Systems). Ten mg of masked Abs are incubated with 50 ng of activated MMP2 overnight at 37° C. The digestions of the masked mAbs are evaluated by SDS-PAGE under reduced condition. It is observed that the molecular weight of heavy chain and light chain for the digested masked Abs are slightly smaller relative to the corresponding undigested pro-antibodies. Upon protease treatment, the molecular weights of the uncapped mAbs are closer to that of the unshielded bispecific Abs and parental unshielded mAbs.

### Example 4: Binding of EGFR and Blocking of EGF by EGFR Hit Antibodies

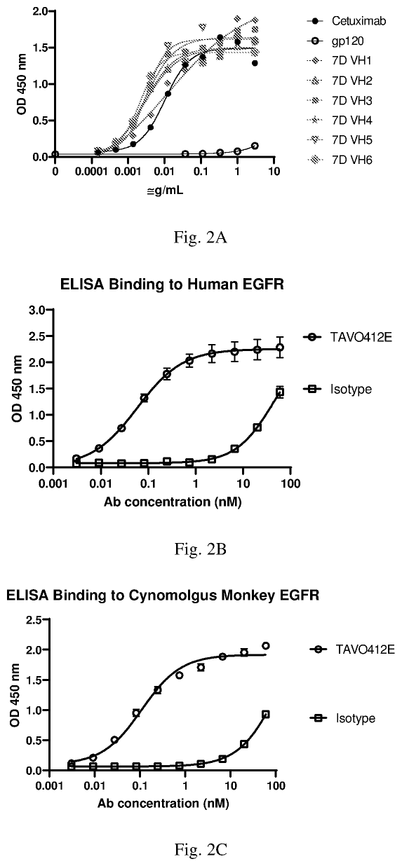

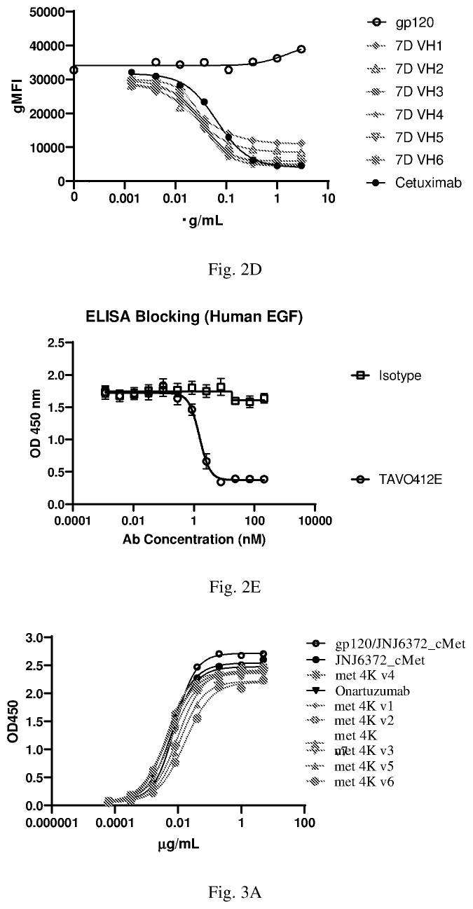

ELISA-based binding assay was employed to evaluate the binding to EGFR by the anti-EGFR mAbs as shown in FIG. 2A. In this assay, human EGFR was coated on the plate and then the EGFR VHO mAbs were added. After washing, the presence of EGFR was detected by an HRP-conjugated anti-His secondary antibody (BioLegend). Results show that the anti-EGFR VHO mAbs (7D VH1-7D VH6) and cetuximab as a positive control can bind EGFR. TAVO412E, an anti-cMET, anti-EGFR, anti-VEGF trispecific antibody, can bind to the human EGFR with an EC50 value of 0.059 nM and to cynomolgus monkey EGFR with an EC50 value of 0.109 nM (FIG. 2B, 2C). The EGF ligand-EGFR blocking assay results are shown in FIG. 2D using the assay format that is illustrated in FIG. 3E. The receptor used was EGFR and the ligand used was EGF at 1 μg/mL. Likewise, these same molecules could block EGF from binding EGFR. TAVO412E could block human EGF from binding to EGFR with an IC50 value of 1.5 nM.

Example 5: Binding of cMET and Blocking of HGF Binding by cMET Hit Antibodies

ELISA-based binding assay was employed to evaluate the binding of anti-cMET mAbs to cMET as shown in FIGS. 3A, 3B, and 3C. Results showed the anti-cMET mAbs bound to cMET. TAVO412E bound to human cMET with an EC50 value of 0.234 nM, to cynomolgus monkey cMET with an EC50 of 0.59 nM. These anti-cMET mAbs also blocked HGF binding to cMET using a protocol as illustrated in FIG. 3E with the data generated as shown in FIGS. 3F and 3G. In this assay, human HGF was coated on the plate and then the cMET mAbs were added. After washing, the presence of cMET was detected by an HRP-conjugated anti-His secondary antibody (BioLegend). The receptor used was cMET and the ligand used was HGF. TAVO412E could block human HGF from binding to cMET with and IC50 value of 8.0 nM.

### Example 6: Binding of VEGF and Blocking of VEGF by TAVO412E

ELISA-based binding assay was employed to evaluate the binding to VEGF by the TAVO412E. In this assay, human VEGF165 was coated on the plate and then the TAVO412E dilutions were added. After washing the non-specific binding, the presence of bound TAVO412E was detected by an HRP-conjugated anti-Fc secondary antibody (BioLegend). TAVO412E bound to human VEGF165 with an EC50 value of 0.085 nM (FIG. 4A) and to cynomolgus monkey VEGF165 with and EC50 value of 0.346 nM (FIG. 4B). Likewise, TAVO412E could block VEGF from binding VEGFR with an IC50 value of 14.8 nM (FIG. 4C).

### Example 7: Design of Trispecific Antibody TAVO412

FIG. 5 shows structural designs for an anti-cMET×anti-EGFR×anti-VEGF multispecific antibody. The anti-cMET×anti-EGFR multispecific antibody as illustrated in FIG. 5 has the EGFR binding arms in black, the cMET binding arms in dark grey, and the VEGF binding arms in light grey as indicated in the figure. FIG. 5A shows that the EGFR binding arms can have a valency of one or two VHO domains. The cMET binding arm can have a valency of one Fab domain. The VEGF binding arm can have a valency of 1-2 domains. The EGFR VHO domains can be on the same heavy chain as N-terminal and C-terminal fusions of the Fc, as tandem Fc fusion molecules on the Fc, or as C terminal fusions on the cMET heavy chain. FIG. 5B shows the EGFR binding arms can have a valency of one or two VHO domains. The cMET binding arm can have a valency of one or two VHO domains on a Fc domain. The cMET VHO domains can be on the same heavy chain as N-terminal fusions of the Fc, as tandem fusion molecules on the Fc, or as N terminal fusions on the EGFR VHO heavy chain fusion molecules.

TAVO412E is a humanized antibody of the IgG1 subclass and is composed of 1 heavy chain with and 1 light chain (kappa) and one chain with 2 nanobody domains fused to a IgG1 Fc with a carboxy terminal single chain Fv. The 3 chains are stabilized by multiple disulfide bonds. TAVO412E is a glycoprotein with the constant region of each heavy chain having a single N-linked glycan site. In order to make the heterodimeric Fc, the clinically validated knob in hole technology was employed for TAVO412. The EGFR-VEGF binding arms have an Fc with the knob mutation T366W and the cMET arms have an Fc with the hole mutations Y407V, L368A, T366S. The heavy chains formed a heterodimer Fc using the knob in hole mutations. To enhance Fc effector function, both the heavy chains have the following clinically validated mutations: F243L, R292P, Y300L, V305I, P396L. TAVO412 has an EGFR arm with tandem anti-EGFR VHO domains, an anti-cMET Fab, and an anti-VEGF scFv domain as shown in a box in FIG. 5A. The linker used to optimize the anti-VEGF scFv was selected for the better activity and stability. The number of G4S linkers to connect the anti-EGFR arms and the anti-VEGF arms were optimized for better stability and developability.

### Example 8. TAVO412E Binding to the Fc Gamma Receptors and C1q

TAVO412E was engineered to have an enhanced ADCC by including the following Fc engineering F243L, R292P, Y300L, V305I, P396L. The Fc-mediated effector functions of antibodies, which include antibody-dependent cellular cytotoxicity (ADCC), antibody-dependent cellular phagocytosis (ADCP), and complement-dependent cytotoxicity (CDC), have been shown to be crucial for the therapeutic efficacy of most clinically approved anti-cancer antibodies. Most of these effector functions are induced via the constant (Fc) region of the antibody, which can interact with complement proteins and specialized Fc-receptors. Surrogate assays that can represent such activity can be shown in TAVO412E binding to CD16A, CD32A, CD64, and C1q. The binding activities of TAVO412 to recombinant CD16A, CD32A, CD64 and purified human C1q were evaluated using ELISA (TAVO412-009). TAVO412 has mutations that enhance the Fc effector function. The antitumor effect of TAVO412 to a considerable extent depends on Fc effector function by binding to complement component 1q (C1q), Fc gamma receptor IIIa (FcγRIIIa or CD16A), and/or Fc gamma receptor I (FcγRI or CD64). To measure the binding to human CD16a, CD32a, or CD64 biotinylated recombinant proteins were added to a plate precoated with streptavidin. Test antibodies were serially diluted and added to the plate. After a 1-hour incubation, the binding was detected by adding HRP-conjugated goat anti-human Fc antibody. The result of color reaction was measured at 450 nm. For the binding to C1q, ELISA plate was coated with serial diluted test antibodies and then added with human C1q. Binding was detected by using the sheep anti-human C1q-HRP labeled antibody, followed by a color reaction. The binding curves of CD16a, CD32a, C1q, and CD64 are shown in FIG. 6. TAVO412E bound to CD16a with an EC50 value of 0.46 nM (FIG. 6A), CD32a with an EC50 value of 2.91 nM (FIG. 6B), CD64 with an EC50 value of 0.16 nM (FIG. 6C), and C1q with an EC50 value of 0.16 nM ((FIG. 6D). Overall, TAVO412E had a better binding to CD16a, CD32A and C1q than the human IgG₁ isotype.

### Example 9. Inhibition of EGF Binding to EGFR in H292 Cells

FIG. 7A shows the assay format of a FACS based assay that was used to characterize the ligand blocking of H292 cells. The anti-cMET×anti-EGFR multispecific antibody was added to compete with 0.2 μg/mL EGF from binding to the cells. The EGF was detected using a AF488 nm labeled rabbit anti-EGF antibody. FIG. 7B shows the gMFI was measured to determine the levels of EGF binding in the presence of the competing mAbs. In this assay, the competitor cMET antibodies did not block EGF binding to the H292 cell lines. The EGFR antibodies could block EGF binding to EGFR. FIG. 8 shows inhibition of EGF binding to EGFR in HCC827 cells. FIG. 8A shows the assay format of a FACS based assay that was used to characterize the ligand blocking of HCC827 cells. The anti-cMET×anti-EGFR multispecific antibody was added to compete with 1 μg/mL EGF from binding to the cells. The EGF was detected using a AF488 nm labeled rabbit anti-EGF antibody. FIG. 8B shows the gMFI was measured to determine the levels of EGF binding in the presence of the competing mAbs. In this assay, the competitor cMET antibodies did not block EGF binding to the HCC827 cell lines. The EGFR antibodies blocked EGF from binding to EGFR on HCC827 cell lines. In this EGFR-EGF blocking experiment, the EC50 values in units of ng/ml for the EGFR×cMet hits were 7D VH6×TV4˜0.63 nM; 7D VH6×EV1˜0.63 nM; 7D VH4×TV4˜0.93 nM; 7D VH4×EV1˜0.81 nM; cetuximab×gp120˜0.60 nM; cetuximab ˜0.33 nM; 7D VH4-Fc ˜0.18 nM; and 7D VH6-Fc ˜0.23 nM.

### Example 10. Inhibition of EGFR Phosphorylation in H1975 Cells Using Western Blot

As shown in FIG. 9, the cMET×EGFR BsAb can inhibit EGFR phosphorylation in NCI-H1975 cells using Western blot. The H1975 cells were seeded to a 12-well plate at 2×10⁵ cells/well. The NCI-H1975 cells have a L858R T790M EGFR and cMET WT genotype. After starvation in non-FBS medium for 18 h at 37° C., the 33.3 nM antibody was added for 1 h and then 500 ng/mL EGF ligand treatment of 30 min. The cells were collected, lysed by cell extraction buffer supplied with phosphatase and protease inhibitors. In FIG. 9A, the top panel have Western blot lanes corresponding to (1) Medium only; (2) EGF only; (3) 7D VH4-Fc; (4) gp120; (5) 7D VH6-Fc; (6) EV1 (SEQ ID NO: 22 and SEQ ID NO: 24); (7) TV4; (8) 7D VH4×EV1; (9) 7D VH4×TV4; (10) 7D VH4×EV1; (11) 7D VH6×TV4; (12) cetuximab×gp120. The integrated values were normalized to the b-actin levels in each lane. All of the 4 candidate BsAbs could inhibit EGFR phosphorylation and had the similar inhibition effect with one armed cetuximab×gp120. In FIG. 9B, the top panel has Western blot lanes corresponding to (1) Medium only; (2) EGF only; (3) 7D VH4×EV1; (4) 7D VH4×TV1; (5) 7D VH6×EV1; (6) 7D VH6×TV4; (7) cetuximab×gp120; (8) gp120; (9) 7D VH4×gp120; (10) 7D VH6×gp120. The integrated values were normalized to the β-actin levels in each lane. All of our 4 candidate BsAbs could inhibit EGFR phosphorylation and had the similar inhibition effect with one armed cetuximab x gp120.

In FIG. 9C, the H292 and HCC827 cells were seeded to a 12 well plate at the density of 2×10⁵ cells per well. The HCC827 cells have a deletion E746 and A750 in EGFR and WT cMET. The H292 cells have a WT EGFR and WT cMET. After starvation with non-FBS medium and incubated 18 h for 37° C., the 33.3 nM antibody was added and incubated for 1 h and then 500 ng/mL EGF ligand treatment of 30 min. The cells were collected, lysed by cell extraction buffer containing phosphatase and protease inhibitors. In FIG. 9C, the results for H292 cells with Western blot lanes corresponding to (1) Medium only; (2) EGF only; (3) 7D VH4×EV1; (4) 7D VH4×TV1; (5) 7D VH6×EV1; (6) 7D VH6×TV4; (7) cetuximab×gp120; (8) gp120; (9) 7D VH4×gp120; (10) 7D VH6×gp120. In FIG. 9D, the results for HCC827 cells with Western blot lanes corresponding to (1) Medium only; (2) EGF only; (3) 7D VH4×EV1; (4) 7D VH4×TV1; (5) 7D VH6×EV1; (6) 7D VH6×TV4; (7) cetuximab×gp120; (8) gp120; (9) 7D VH4×gp120; (10) 7D VH6×gp120. The integrated values were normalized to the β-actin levels in each lane. All of 4 candidate BsAbs could inhibit EGFR phosphorylation and had the similar inhibitory effect with one armed cetuximab×gp120 in H292 cells, but not for HCC827 cells.

### Example 11. Demonstration of TAVO412E Utility in a Non-Small Cell Lung Cancer Cell Line HCC827

FIG. 10 demonstrated TAVO412E cell binding, blocking of EGF from binding to EGFR on HCC827 cells, and blocking of HGF from binding to cMET on HCC827 cells. In the cell binding experiments, the HCC827 cells were seeded into a 96-well-plate at 50,000 cells per well. Serial dilutions of antibody were added and incubated for 1h in the dark at 4° C. After washing, Alexa Fluor 647 Fcγ fragment specific goat anti-human IgG was used for detection on a Beckman flow cytometer at 638 nm of excitation and 660 nm of emission. In the blocking of EGF from binding to EGFR on HCC827 cells experiments, the cells were harvested and plated into 96-well-plate at 50,000 cells/well. Serial dilutions of antibody were added and incubated for 1h in the dark at 4° C. After washing, cells were incubated with EGF for 1h in the dark at 4° C. Rabbit polyclonal anti-human EGF and Alexa Fluor 488 anti-rabbit IgG1 were used for detection on a Beckman flow cytometer at 488 nm of excitation and 525 nm of emission. In the blocking of HGF from binding to cMET on HCC827 experiments, the cells were harvested and plated into 96-well-plate at 50,000 cells/well. Serial antibody dilutions were added and incubated for 0.5 h in the dark at 4° C. 0.7 μg/mL of biotinylated HGF was added and incubated for 0.5 h in the dark at 4° C. following washing. Streptavidin-AF488 was used to detect biotinylated HGF on a Beckman flow cytometer at 488 nm of excitation and 525 nm of emission. In FIG. 10A-C, the y axes represented the gMFI that reflected the binding levels on HCC827 cells, and the x axes represented the concentration of the test reagents. FIG. 10A shows that TAVO412E had an EC50 value for binding to HCC827 cells of 1.04 nM. The isotype mAb had no binding to HCC827 cells. FIG. 10B shows that TAVO412E had an IC50 value for blocking the binding of EGF to EGFR on HCC827 cells of 2.56 nM. The isotype mAb had no blocking of EGF binding to EGFR on HCC827 cells. FIG. 10C shows that TAVO412E had an IC50 value for blocking the binding of HGF to cMET on HCC827 cells of 0.28 nM. The isotype mAb had no blocking of HGF binding to cMET on HCC827 cells.

Example 12. TAVO412E Inhibition of the Phosphorylation of EGFR and cMET in H292 and HCC827 Cells.

In this assay, either H292 or HCC827 cells were seeded into a 96 well plate at the density of 40,000 cells per well and incubated overnight. Cells were starved in serum-free medium for 24 h. Serial antibody dilutions were added into the plate and incubated for 1 h at 37° C., then ligands (HGF or HGF+EGF) were added for a 15 min incubation at 37° C. Cells were lysed with lysis buffer with phosphor-inhibitor, and the cell lysis was transferred to a 384-well plate and incubated with HTRF antibodies for 4 h at RT. The plate was read on a Decan Spark plate reader at 320/615, 320/665. The phosphorylation ratio percentage (%) was determined for each drug/concentration, and a dose response curve was generated. In FIG. 11A-D, the y axes are shown as values of percent EGFR phosphorylation as noted in the control mAb and the x axes are concentrations of the test articles. FIG. 11A shows TAVO412E inhibited EGFR phosphorylation in H292 cells in the presence of EGF with an IC50 value of 0.79 nM. The isotype mAb did not inhibit EGFR phosphorylation. FIG. 11B shows TAVO412E inhibited EGFR phosphorylation in H292 cells in the presence of EGF and HGF with an IC50 value of 0.78 nM. The isotype mAb did not inhibit EGFR phosphorylation. FIG. 11C shows TAVO412E inhibited cMET phosphorylation in HCC827 cells in the presence of HGF with an IC50 value of 1.41 nM. The isotype mAb did not inhibit cMET phosphorylation. FIG. 11D shows TAVO412E inhibited cMET phosphorylation in HCC827 cells in the presence of HGF and EGF with an IC50 value of 1.99 nM. The isotype mAb did not inhibit cMET phosphorylation.

### Example 13. TAVO412E Inhibition of the Proliferation of HCC827 Cells

HCC827 cells were seeded at 10,000 cells per well in a 96-well plate and incubated overnight. Then the cells were starved in serum-free medium followed and incubated for 24 h. The next day, serial antibody dilutions were added into the plate. After 3-day incubation, PrestoBlue reagent was added for cell viability detection using Tecan Spark microplate reader at 560 nm and 590 nm. Surviving rate was calculated as (Fluorescence of test antibody-Fluorescence of medium control)/(Fluorescence of non-treated cell control-Fluorescence of medium control). FIG. 12A shows TAVO412E inhibited the proliferation of HCC827 cells with an IC50 value of 1.76 nM. The isotype mAb did not inhibit the proliferation of HCC827 cells. FIG. 12B shows TAVO412E inhibited the proliferation of HCC827 cells in the presence of EGF and HGF with an IC50 value of 1.39 nM. The isotype mAb did not inhibit the proliferation of HCC827 cells.

Example 14. In Vitro Inhibition of cMET Phosphorylation in H1975, HCC827, H292 Cells Using Western Blot

In FIG. 13, the cells were seeded to a 12-well plate at 2×10⁵ cells/well. After starvation in non-FBS medium for 18 h at 37° C., the cells were incubated with 33.3 nM antibody for 1 h and then were treated with 500 ng/mL HGF ligand treatment of 30 min. The cells were collected, lysed by cell extraction buffer supplied with phosphatase and protease inhibitors. FIG. 13A shows the results for HCl-H1975; FIG. 13B Results for HCC827; FIG. 13C shows the results for H292 cells. The Western blot lanes corresponded to (1) Medium only; (2) HGF only; (3) 7D VH4×EV1; (4) 7D VH4×TV1; (5) 7D VH6×EV1; (6) 7D VH6×TV4; (7) cetuximab×gp120; (8) gp120; (9) 7D VH4×gp120; (10) 7D VH6×gp120. The 4 BsAbs displayed more significant inhibition effect than their monovalent parental Abs in HCC827, H292, and NCI-H1975 cells.

### Example 15. In Vitro Fc Effector Function of TAVO412E on HCC827 Cells

For the reporter assay as shown in FIGS. 14A and 14B, the target cells (2×10⁴ cells per well) and Jurkat-CD16A-V158 or Jurkat-CD32A-H131 reporter cells (2×10⁵ cells per well) were harvested and co-cultured at a E: T ratio of 10:1 in a 96-well plate. Serial dilutions of a test antibody were dispensed into the plate to incubate at 37° C. for 6 hours. After incubation, the cell supernatants were transferred to a white wall plate and Bio-Lite reagent was added to each well. The luminescence was measured using a Decan Spark®. To determine ADCC activity in HCC827 cells as shown in FIG. 14C, the target cells were seeded into a round bottom 96-well plate at 1×10⁴ cells per well and cultured with the serial dilutions of test antibodies at 37° C. for 15 minutes first, then the frozen PBMCs were recovered and added to the plate at a E: T ratio of 50:1. The plates were centrifuged to ensure the contact between effector and target cells and incubated at 37° C. for 4 h. After centrifugation, the cell supernatants were transferred to a new flat bottom plate. LDH kit was used to test cell lysis. The absorbance was read at 492 nm and 650 nm using a Decan Spark®. ADCC % was calculated as (Experimental release−Spontaneous release)/(Maximal release −Spontaneous release). To determine ADCP-Macrophage killing activity in NSCLC Cancer cell lines as shown in FIG. 14D, the monocytes were isolated from PBMCs and were induced with the cytokines of 25 ng/ml of MCSF and 50 ng/ml of IFNγ to differentiate into the macrophages. The target cells were harvested and stained with CSFE. The differentiated macrophages (1×10⁵ cells per well) and the labelled target cells (5×10⁴ cells per well) were co-cultured at E: T ratio of 2:1 in a 96-well plate, the serial dilutions of test antibody were added and incubated. After a 24 h incubation, the cells were collected and stained with Alexa647-labeled CD14 and CD11b antibodies for 30 min. After washing, cells were measured on a Beckman flow cytometry at 638 nm and 660 nm. Percent killing was determined using the equation as ((average % FITC+AF647-of [lowest mAb] for each antibody)-% FITC+AF647-sample)/(average % FITC+AF647-of [lowest mAb] for each antibody). For the CDC experiments as shown in FIG. 14E, the cells were harvested and seeded in a 96-well plate at the optimized cell density in basic medium. The cells were incubated with the serial antibody dilutions were added and incubated for 1 h at RT. The rabbit serum was aliquoted to the plate and incubated at 37° C. for 1 h. After incubation, the cell supernatants were transferred to a new plate and LDH kit was used to detect cell lysis. The absorbance values were read on a Decan Spark® at 492 nm and 650 nm. The lysis % was calculated by dividing the absorbance value of the sample by that of the control. The dose-response curve was generated by GraphPad Prism 9.3.1. FIG. 14A shows TAVO412E had ADCC reporter activity on HCC827 cells with an IC50 value of 0.2 nM. The isotype mAb did not have ADCP reporter activity of HCC827 cells. FIG. 14B shows TAVO412E had ADCP reporter activity on HCC827 cells with an IC50 value of ˜1 nM. The isotype mAb did not have ADCP reporter activity of HCC827 cells. FIG. 14C shows TAVO412E had ADCC killing activity on HCC827 cells with an IC50 value of 0.12 nM. The isotype mAb did not have ADCP reporter activity of HCC827 cells. FIG. 14D shows TAVO412E induced ADCP killing activity on HCC827 cells with an IC50 value of 0.16 nM. The isotype mAb did not have ADCP reporter activity of HCC827 cells. FIG. 14E shows TAVO412E had CDC killing activity on HCC827 cells with an IC50 value of 3.76 nM. The isotype mAb did not have ADCP reporter activity of HCC827 cells.

### Example 16. In Vivo Anti-Tumor Activity of TAVO412E on the Non-Small Cell Lung Cancer Cell Line H1975

The following guidelines were used in the xenograft models experiments. All the protocols and amendment(s) or procedures involving the care and use of animals were reviewed and approved by the GenePharma Institutional Animal Care and Use Committee (IACUC) prior to conducting the studies. Tumor cells were transplanted subcutaneously into female Balb/c nude mice and treatments were started when the mean tumor volume reached 100-200 mm³. Testing antibodies were injected i.p. at a twice weekly dosing regimen. Tumor growth and body weight was measured twice weekly until endpoint and tumor volume was determined as length×width²×0.5. Tumor growth inhibition (TGI) was calculated as TGI %=1−T/C, where T and C were the mean tumor volume of the treated and control groups on the last day, respectively.

The following protocol were used for the in vivo receptor degradation experiments. Mice bearing established tumors were treated with two doses of vehicle control. Twenty-four hours post the 2^{nd} dose, tumors were harvested and flash frozen in liquid nitrogen. Tumors were lysed in ice-cold RIPA buffer containing protease and phosphatase inhibitor cocktail using homogenizer. Lysates were cleared by centrifugation, and protein concentrations were determined by BCA Protein Assay. Protein samples were resolved by SDS-PAGE and transferred to PVDF membranes. Membranes were blocked in 5% BSA blocking buffer for 1 hour at room temperature and incubated with the appropriate primary antibodies overnight at 4° C. ECL detection was performed by incubating the membrane and ECL regents. e-BLOT WB IMAGER was used to acquire image. The western blot images were analyzed with image J software. Average total protein relative to loading control (GAPDH) was graphed and statistical analysis performed using GraphPad Prism version 9.3.1.

FIG. 15A shows H1975 tumor growth inhibition at day 13 of 42% at 1 mg/kg, 76% at 3 mg/kg, and 94% at 10 mg/kg. TAVO412E had a dose dependent tumor growth inhibition in H1975 cells. FIG. 15B shows that TAVO412E induced degradation of EGFR in the tumors in the in vivo H1975 xenograft model as well as reduction of EGFR phosphorylation. FIG. 15C shows that TAVO412E induced degradation of cMET in the tumors in the in vivo H1975 xenograft model as well as reduction of cMET phosphorylation. FIG. 15D shows the bar graph representation of the results for the control isotype mAb and TAVO412E in FIGS. 15B and C. TAVO412E decreased the levels of the total and phosphorylated forms of cMET and EGFR in the in vivo H1975 xenograft model experiment.

### Example 17. In Vivo Anti-Tumor Activity of TAVO412E on the Non-Small Cell Lung Cancer Cell Line HCC827

The xenograft and receptor degradation protocols are outlined in Example 16. FIG. 16A shows HCC827 tumor growth inhibition at day 13 of 45% at 1 mg/kg, 79% at 3 mg/kg, and 94% at 10 mg/kg. TAVO412E had a dose dependent tumor growth inhibition in HCC827 cells. FIG. 16B shows that TAVO412E induced degradation of EGFR and cMET in the tumors in the in vivo HCC827 xenograft model experiment. FIG. 16C shows the bar graph representation of the results for the control isotype mAb and TAVO412E in FIG. 16B. TAVO412E decreased the levels of the total forms of cMET and EGFR in the in vivo HCC827 xenograft model experiment.

### Example 18. In Vitro Anti-Tumor Activity of TAVO412E on the Triple Negative Breast Cancer Cell Line MDA-MB-468

FIG. 17A shows TAVO412E binding to MDA-MB-468 with an EC50 value for binding of 1.11 nM. TAVO412E had a dose dependent tumor growth inhibition in H1975 cells. FIG. 17B shows TAVO412E inhibited human EGFR phosphorylation in MDA-MB-468 cells in the presence of human EGF with an IC50 value of 9.08 nM. The isotype mAb did not inhibit human EGFR phosphorylation. FIG. 17C shows TAVO412E inhibited human EGFR phosphorylation in MDA-MB-468 cells in the presence of human EGF and human HGF with an IC50 value of 8.50 nM. The isotype mAb did not inhibit EGFR phosphorylation.

### Example 19. In Vitro Anti-Tumor Activity of TAVO412E on the Triple Negative Breast Cancer Cell Line MDA-MB-231

For the ADCC experiments, frozen PBMCs were recovered and cultured overnight. The next day, the target cells were seeded into a round bottom 96-well plate and cultured with the serial dilutions of test antibodies at 37° C. for 15 minutes first, then the PBMCs were added at a E: T ratio of 50:1. The plates were centrifuged to ensure the contact between effector and target cells and incubated at 37° C. for 4 h. After centrifugation, the cell supernatants were transferred to a new flat bottom plate. LDH kit was used to test cell lysis. The absorbance was read at 492 nm and 650 nm using a Decan Spark®. ADCC % was calculated as (Experimental release-Spontaneous release)/(Maximal release-Spontaneous release). For the ADCP experiments, the monocytes were isolated from PBMCs and were treated with the cytokines of MCSF and IFNγ to differentiate into the macrophages. The target cells were harvested and stained with CSFE. The differentiated macrophages and the labelled target cells were co-cultured at E: T ratio of 2:1, the serial dilutions of test antibody were added and incubated. After a 24 h incubation, the cells were collected and stained with Alexa647-labeled CD14 and CD11b antibodies for 30 min. After washing, cells were measured on a Beckman flow cytometry at 638 nm and 660 nm. Percent killing was determined using the equation as ((average % FITC+AF647-of [lowest mAb] for each antibody)-% FITC+AF647-sample)/(average % FITC+AF647-of [lowest mAb] for each antibody). The results of three independent measurements were then collected, graphed, and processed in GraphPad Prism 9.3.1. For the CDC experiments, the cells were harvested and seeded in a 96-well plate at the optimized cell density in basic medium. The serial antibody dilutions were added and incubated for 1 h at RT. The rabbit serum was aliquoted to the plate and incubated at 37° C. for 1-4 h. After incubation, the cell supernatants were transferred to a new plate and LDH kit was used to test cell lysis. The absorbance values were read on a Decan Spark® at 492 nm and 650 nm. The lysis % was calculated by dividing the absorbance value of the sample by that of the control. The dose-response curve was generated by GraphPad Prism 9.3.1.

FIG. 18A shows TAVO412E bound to MDA-MB-231 with an EC50 value for binding of 0.37 nM. FIG. 18B shows TAVO412E had ADCP reporter activity on MDA-MB-231 cells with an EC50 value of 0.087 nM. The isotype mAb did not have ADCP reporter assay response. FIG. 18C shows TAVO412E had ADCP killing of MDA-MB-231 cells with an EC50 value of 0.156 nM. The isotype mAb did not have an ADCP killing response. FIG. 18D shows TAVO412E had ADCC reporter activity on MDA-MB-231 cells with an EC50 value of 0.18 nM. The isotype mAb did not have ADCP reporter assay response. FIG. 18E shows TAVO412E had ADCC killing of MDA-MB-231 cells with an EC50 value of 0.13 nM. The isotype mAb did not have an ADCC killing response. FIG. 18F shows TAVO412E had CDC killing of MDA-MB-231 cells with an EC50 value of 1.22 nM. The isotype mAb did not have a CDC killing response.

### Example 20. In Vivo Anti-Tumor Activity of TAVO412E on the Triple Negative Breast Cancer Cell Line MDA-MB-231

The xenograft and receptor degradation protocols are outlined in Example 16. FIG. 19A shows MDA-MB-231 tumor growth inhibition at day 20 of 62% at 10 mg/kg dosing. FIG. 19B shows that TAVO412E induced degradation of EGFR and cMET in the tumors in the in vivo MDA-MB-231 xenograft model experiment. FIG. 19C shows the bar graph representation of the results for the control isotype mAb and TAVO412E in FIG. 19B. TAVO412E decreased the levels of the total forms of cMET and EGFR in the in vivo MDA-MB-231 xenograft model experiment.

Example 21. In Vitro TAVO412E Utility in Gastric Cancer Cell Lines SNU-5 and MKN-45 as Shown by Cell Binding, Blocking of HGF from Binding to cMET on MKN45 Cells, and Proliferation Inhibition of SNU-5 Cells

For the HGF blocking in MKN45 experiments, cells were harvested and plated into 96-well-plate at 50,000 cells/well. Serial dilutions of HGF and 0.1 μg/mL of test antibody were added in sequence and incubated for 1h in the dark at 4° C. After washing, Alexa Fluor 488 anti-rabbit IgG1 were used to detect the test antibody on a Beckman flow cytometer at 488 nm of excitation and 525 nm of emission. In the proliferation inhibition study, 3k SNU-5 cells/well with no starvation were put into the 96 well plate. The cells were treated with test articles 6 days and cell proliferation/survival was measured using Alamar blue. FIG. 20A shows that TAVO412E had an EC50 value for binding to MKN45 cells of 1.78 nM. The isotype mAb had no binding to MKN45 cells. FIG. 20B shows that TAVO412E had an IC50 value for blocking the binding of HGF to cMET on MKN45 cells of 0.28 nM. The isotype mAb had no blocking of HGF binding to cMET on MKN45 cells. FIG. 20C shows that TAVO412E had an EC50 value for binding to SNU-5 cells of 1.99 nM. The isotype mAb had binding to SNU-5 cells. FIG. 20D shows that TAVO412E had an IC50 value for the inhibition of proliferation of SNU-5 cells of 2.66 nM. The isotype mAb had no inhibition of proliferation of SNU-5 cells.

### Example 22. In Vitro Anti-Tumor Activity of TAVO412E on the Gastric Cancer Cell Line SNU-5

The experimental protocols have been described for FIG. 18. FIG. 21A shows TAVO412E had ADCC reporter activity on SNU-5 cells with an EC50 value of 0.18 nM. The isotype mAb did not have ADCC reporter assay response. FIG. 21B shows TAVO412E had ADCP reporter activity on SNU-5 cells with an EC50 value of 0.20 nM. The isotype mAb did not have ADCP reporter assay response. FIG. 21C shows TAVO412E had CDC killing of SNU-5 cells with an EC50 value of 1.19 nM. The isotype mAb did not have a CDC killing response.

### Example 23. In Vivo Anti-Tumor Activity of TAVO412E on the Gastric Cancer Cell Line MKN45

The xenograft and receptor degradation protocols are outlined in Example 16. FIG. 22A shows MKN-45 tumor growth inhibition at day 21 of 70% at 3 mg/kg dosing. FIG. 22B shows that TAVO412E induced degradation of EGFR and cMET in the tumors in the in vivo MKN45 xenograft model experiment. FIG. 22C shows the bar graph representation of the results for the control isotype mAb and TAVO412E in FIG. 22B. TAVO412E decreased the levels of the total forms of cMET and EGFR in the tumors excised from the in vivo MKN45 xenograft model experiment.

### Example 24. In Vitro TAVO412E Utility in Pancreatic Ductal Adenocarcinoma Cancer Cell Line BxPC-3 as Shown by Cell Binding, ADCC Reporter Assay, and ADCP Reporter Assay

FIG. 23A shows that TAVO412E had an EC50 value for binding to BxPC-3 cells of 0.90 nM. The isotype mAb had no binding to BxPC-3 cells. FIG. 23B shows that TAVO412E had an EC50 value for ADCC reporter assay on BxPC-3 cells of 0.20 nM. The isotype mAb had no ADCC reporter assay activation on BxPC-3 cells. FIG. 23C shows that TAVO412E had an EC50 value for ADCP reporter assay on BxPC-3 cells of 0.65 nM. The isotype mAb had no ADCP reporter assay activation on BxPC-3 cells.

Example 25. In Vitro Inhibition of the Phosphorylation of EGFR and cMET in BxPC-3 Cells

The experiment was done analogously as what was described for FIG. 11. In FIG. 24A-D, the y axes are shown as values of percent receptor phosphorylation as noted in the control mAb and the x axes were concentrations of the test articles. FIG. 24A shows TAVO412E inhibited EGFR phosphorylation in BxPC-3 cells in the presence of recombinant human EGF with an IC50 value of 3.45 nM. The isotype mAb did not inhibit EGFR phosphorylation. FIG. 24B shows TAVO412E inhibited cMET phosphorylation in BxPC-3 cells in the presence of recombinant human HGF with an IC50 value of 1.18 nM. The isotype mAb did not inhibit cMET phosphorylation. FIG. 24C shows TAVO412E inhibited EGFR phosphorylation in BxPC-3 cells in the presence of recombinant human EGF and recombinant human HGF with an IC50 value of 1.13 nM. The isotype mAb did not inhibit EGFR phosphorylation. FIG. 24D shows TAVO412E inhibiting cMET phosphorylation in BxPC-3 cells in the presence of recombinant human EGF and recombinant human HGF with an IC50 value of 0.44 nM. The isotype mAb did not inhibit cMET phosphorylation.

### Example 26. In Vivo Anti-Tumor Activity of TAVO412E on the Pancreatic Ductal Adenocarcinoma Cancer Cell Line BxPC-3

The xenograft and receptor degradation protocols are outlined in Example 16. FIG. 25A shows TAVO412 treatment results in BxPC-3 tumor growth inhibition of 80% at 10 mg/kg dosing at day 34. FIG. 25B shows that TAVO412E induced degradation of EGFR and cMET in the tumors in the in vivo BxPC-3 xenograft model experiment. FIG. 25C shows the bar graph representation of the results for the control isotype mAb and TAVO412E in FIG. 25B. TAVO412E decreased the levels of the total forms of cMET and EGFR in the tumors excised from the in vivo BxPC-3 xenograft model experiment.

### Example 27. Anti-Tumor Activity of TAVO412E on the Liver Cancer Cell Line HCC9810 In Vitro, Triple Negative Breast Cancer Cell Line HCC70 In Vivo, and Head and Neck Cancer Cell Line FaDu In Vivo

The xenograft and receptor degradation protocols are outlined in Example 16. FIG. 26A shows TAVO412E had ADCC activity on HCC9810 cell line with an EC50 value of 0.098 nM. FIG. 26B shows TAVO412 treatment resulted in HCC-70 tumor growth inhibition of 26% at 10 mg/kg dosing at day 21. FIG. 26C shows TAVO412 treatment resulted in FaDu tumor growth inhibition of 95% at 10 mg/kg dosing at day 21.

### Example 28. TAVO412E In Vitro Anti-Tumor Activity in Head and Neck Esophageal Squamous Cell Carcinoma Cancer Cell Line KYSE-150 as Shown by Cell Binding, ADCC Reporter Assay, and ADCC Killing Assay

FIG. 27A shows that TAVO412E had an EC50 value for binding to KYSE-150 cells of 0.39 nM. The isotype mAb had no binding to KYSE-150 cells. FIG. 27B shows that TAVO412E had an EC50 value for ADCC reporter assay on KYSE-150 cells of 0.15 nM. The isotype mAb had no ADCC reporter assay activation on KYSE-150 cells. FIG. 27C shows that TAVO412E had an EC50 value for ADCC killing assay on KYSE-150 cells of 0.038 nM. The isotype mAb had no ADCC killing response on KYSE-150 cells.

### Example 29. TAVO412 Anti-Tumor In Vitro Activity in Mesothelioma Cancer Cell Line NCI-H226 as Shown by Cell Binding, ADCC Reporter Assay, and ADCC Killing Assay.

FIG. 28A shows that TAVO412E had an EC50 value for binding to NCI-H226 cells of 0.78 nM. The isotype mAb had no binding to NCI-H226 cells. FIG. 28B shows that TAVO412E had an EC50 value for ADCC reporter assay on NCI-H226 cells of 0.17 nM. The isotype mAb had no ADCC reporter assay activation on NCI-H226 cells. FIG. 28C shows that TAVO412E had an EC50 value for ADCC killing assay on NCI-H226 cells of 0.025 nM. The isotype mAb had no ADCC killing response on NCI-H226 cells.

### Example 30. TAVO412 Anti-Tumor In Vitro Activity in Colorectal Cancer Cell Line HT-29 as Shown by Cell Binding, ADCC Reporter Assay, and ADCC Killing Assay

FIG. 29A shows that TAVO412E had an EC50 value for binding to HT-29 cells of 0.23 nM. The isotype mAb had no binding to HT-29 cells. FIG. 28B shows that TAVO412E had an EC50 value for ADCC reporter assay on HT-29 cells of 0.078 nM. The isotype mAb had no ADCC reporter assay activation on HT-29 cells. FIG. 27C shows that TAVO412E had an EC50 value for ADCC killing assay on HT-29 cells of 0.023 nM. The isotype mAb had no ADCC killing response on HT-29 cells.

### REFERENCES

Banys-Paluchowski, M., I. Witzel, B. Aktas, P. A. Fasching, A. Hartkopf, W. Janni, S. Kasimir-Bauer, K. Pantel, G. Schon, B. Rack, S. Riethdorf, E. F. Solomayer, T. Fehm and V. Muller (2019). “The prognostic relevance of urokinase-type plasminogen activator (uPA) in the blood of patients with metastatic breast cancer.” Sci Rep 9 (1): 2318.
Bean, J., C. Brennan, J. Y. Shih, G. Riely, A. Viale, L. Wang, D. Chitale, N. Motoi, J. Szoke, S. Broderick, M. Balak, W. C. Chang, C. J. Yu, A. Gazdar, H. Pass, V. Rusch, W. Gerald, S. F. Huang, P. C. Yang, V. Miller, M. Ladanyi, C. H. Yang and W. Pao (2007). “MET amplification occurs with or without T790M mutations in EGFR mutant lung tumors with acquired resistance to gefitinib or erlotinib.” Proc Natl Acad Sci USA 104 (52): 20932-20937.
Birchmeier, C., W. Birchmeier, E. Gherardi and G. F. Vande Woude (2003). “Met, metastasis, motility and more.” Nat Rev Mol Cell Biol 4 (12): 915-925.
Bolt, S., E. Routledge, I. Lloyd, L. Chatenoud, H. Pope, S. D. Gorman, M. Clark and H. Waldmann (1993). “The generation of a humanized, non-mitogenic CD3 monoclonal antibody which retains in vitro immunosuppressive properties.” Eur J Immunol 23 (2): 403-411.
Chen, N., W. Fang, J. Zhan, S. Hong, Y. Tang, S. Kang, Y. Zhang, X. He, T. Zhou, T. Qin, Y. Huang, X. Yi and L. Zhang (2015). “Upregulation of PD-L1 by EGFR Activation Mediates the Immune Escape in EGFR-Driven NSCLC: Implication for Optional Immune Targeted Therapy for NSCLC Patients with EGFR Mutation.” J Thorac Oncol 10 (6): 910-923.
Chen, X., J. L. Zaro and W. C. Shen (2013). “Fusion protein linkers: property, design and functionality.” Adv Drug Deliv Rev 65 (10): 1357-1369.
Chothia, C. and A. M. Lesk (1987). “Canonical structures for the hypervariable regions of immunoglobulins.” J Mol Biol 196 (4): 901-917.
Chu, S. Y., I. Vostiar, S. Karki, G. L. Moore, G. A. Lazar, E. Pong, P. F. Joyce, D. E. Szymkowski and J. R. Desjarlais (2008). “Inhibition of B cell receptor-mediated activation of primary human B cells by coengagement of CD19 and FcgammaRIIb with Fc-engineered antibodies.” Mol Immunol 45 (15): 3926-3933.
Cui, J. J., H. Shen, M. Tran-Dube, M. Nambu, M. McTigue, N. Grodsky, K. Ryan, S. Yamazaki, S. Aguirre, M. Parker, Q. Li, H. Zou and J. Christensen (2013). “Lessons from (S)-6-(1-(6-(1-methyl-1H-pyrazol-4-yl)-[1,2,4]triazolo[4,3-b]pyridazin-3-yl)ethyl) quinoline (PF-04254644), an inhibitor of receptor tyrosine kinase c-Met with high protein kinase selectivity but broad phosphodiesterase family inhibition leading to myocardial degeneration in rats.” J Med Chem 56 (17): 6651-6665.
Dall'Acqua, W. F., P. A. Kiener and H. Wu (2006). “Properties of human IgG1s engineered for enhanced binding to the neonatal Fc receptor (FcRn).” J Biol Chem 281 (33): 23514-23524.
Diebolder, C. A., F. J. Beurskens, R. N. de Jong, R. I. Koning, K. Strumane, M. A. Lindorfer, M. Voorhorst, D. Ugurlar, S. Rosati, A. J. Heck, J. G. van de Winkel, I. A. Wilson, A. J. Koster, R. P. Taylor, E. O. Saphire, D. R. Burton, J. Schuurman, P. Gros and P. W. Parren (2014). “Complement is activated by IgG hexamers assembled at the cell surface.” Science 343 (6176): 1260-1263.
Engelman, J. A., K. Zejnullahu, T. Mitsudomi, Y. Song, C. Hyland, J. O. Park, N. Lindeman, C. M. Gale, X. Zhao, J. Christensen, T. Kosaka, A. J. Holmes, A. M. Rogers, F. Cappuzzo, T. Mok, C. Lee, B. E. Johnson, L. C. Cantley and P. A. Janne (2007). “MET amplification leads to gefitinib resistance in lung cancer by activating ERBB3 signaling.” Science 316 (5827): 1039-1043.
Fan, J. B., W. Liu, X. H. Zhu, K. Yuan, D. W. Xu, J. J. Chen and Z. M. Cui (2015). “EGFR-AKT-mTOR activation mediates epiregulin-induced pleiotropic functions in cultured osteoblasts.” Mol Cell Biochem 398 (1-2): 105-113.
Folkman, J. (1995). “Angiogenesis in cancer, vascular, rheumatoid and other disease.” Nat Med 1 (1): 27-31.
Folkman, J. and Y. Shing (1992). “Angiogenesis.” J Biol Chem 267 (16): 10931-10934.
Fukumura, D., R. Xavier, T. Sugiura, Y. Chen, E. C. Park, N. Lu, M. Selig, G. Nielsen, T. Taksir, R. K. Jain and B. Seed (1998). “Tumor induction of VEGF promoter activity in stromal cells.” Cell 94 (6): 715-725.
Gunasekaran, K., M. Pentony, M. Shen, L. Garrett, C. Forte, A. Woodward, S. B. Ng, T. Born, M. Retter, K. Manchulenko, H. Sweet, I. N. Foltz, M. Wittekind and W. Yan (2010). “Enhancing antibody Fc heterodimer formation through electrostatic steering effects: applications to bispecific molecules and monovalent IgG.” J Biol Chem 285 (25): 19637-19646.
Hinton, P. R., J. M. Xiong, M. G. Johlfs, M. T. Tang, S. Keller and N. Tsurushita (2006). “An engineered human IgG₁ antibody with longer serum half-life.” J Immunol 176 (1): 346-356.
Holmgren, L., M. S. O'Reilly and J. Folkman (1995). “Dormancy of micrometastases: balanced proliferation and apoptosis in the presence of angiogenesis suppression.” Nat Med 1 (2): 149-153.
Horak, E. R., R. Leek, N. Klenk, S. LeJeune, K. Smith, N. Stuart, M. Greenall, K. Stepniewska and A. L. Harris (1992). “Angiogenesis, assessed by platelet/endothelial cell adhesion molecule antibodies, as indicator of node metastases and survival in breast cancer.” Lancet 340 (8828): 1120-1124.
Jiang, W., T. Li, J. Guo, J. Wang, L. Jia, X. Shi, T. Yang, R. Jiao, X. Wei, Z. Feng, Q. Tang and G. Ji (2021). “Bispecific c-Met/PD-L1 CAR-T Cells Have Enhanced Therapeutic Effects on Hepatocellular Carcinoma.” Front Oncol 11:546586.
Kaumaya, P. T. P. and K. C. Foy (2012). “Peptide vaccines and peptidomimetics targeting HER and VEGF proteins may offer a potentially new paradigm in cancer immunotherapy.” Future Oncol. 8 (8): 961-987.
Kennedy, S. P., J. F. Hastings, J. Z. Han and D. R. Croucher (2016). “The Under-Appreciated Promiscuity of the Epidermal Growth Factor Receptor Family.” Front Cell Dev Biol 4:88.
Kinder, M., A. R. Greenplate, K. D. Grugan, K. L. Soring, K. A. Heeringa, S. G. McCarthy, G. Bannish, M. Perpetua, F. Lynch, R. E. Jordan, W. R. Strohl and R. J. Brezski (2013). “Engineered protease-resistant antibodies with selectable cell-killing functions.” J Biol Chem 288 (43): 30843-30854.
Klagsbrun, M. and P. A. D′Amore (1991). “Regulators of angiogenesis.” Annu Rev Physiol 53:217-239.
Labrijn, A. F., J. I. Meesters, B. E. de Goeij, E. T. van den Bremer, J. Neijssen, M. D. van Kampen, K. Strumane, S. Verploegen, A. Kundu, M. J. Gramer, P. H. van Berkel, J. G. van de Winkel, J. Schuurman and P. W. Parren (2014). “Efficient generation of stable bispecific IgG₁ by controlled Fab-arm exchange.” Proc Natl Acad Sci USA 110 (13): 5145-5150.
Lee, D., E. S. Sung, J. H. Ahn, S. An, J. Huh and W. K. You (2015). “Development of antibody-based c-Met inhibitors for targeted cancer therapy.” Immunotargets Ther 4:35-44.
Lefranc, M. P., C. Pommie, M. Ruiz, V. Giudicelli, E. Foulquier, L. Truong, V. Thouvenin-Contet and G. Lefranc (2003). “IMGT unique numbering for immunoglobulin and T cell receptor variable domains and Ig superfamily V-like domains.” Dev Comp Immunol 27 (1): 55-77.
Lin, W. W., Y. C. Lu, C. H. Chuang and T. L. Cheng (2020). “Ab locks for improving the selectivity and safety of antibody drugs.” J Biomed Sci 27 (1): 76.
Macchiarini, P., G. Fontanini, M. J. Hardin, F. Squartini and C. A. Angeletti (1992). “Relation of neovascularisation to metastasis of non-small-cell lung cancer.” Lancet 340 (8812): 145-146.
McDermott, U., R. V. Pusapati, J. G. Christensen, N. S. Gray and J. Settleman (2010). “Acquired resistance of non-small cell lung cancer cells to MET kinase inhibition is mediated by a switch to epidermal growth factor receptor dependency.” Cancer Res 70 (4): 1625-1634.
Pawelczyk, K., A. Piotrowska, U. Ciesielska, K. Jablonska, N. Gletzel-Plucinska, J. Grzegrzolka, M. Podhorska-Okolow, P. Dziegiel and K. Nowinska (2019). “Role of PD-L1 Expression in Non-Small Cell Lung Cancer and Their Prognostic Significance according to Clinicopathological Factors and Diagnostic Markers.” Int J Mol Sci 20 (4).
Petkova, S. B., S. Akilesh, T. J. Sproule, G. J. Christianson, H. Al Khabbaz, A. C. Brown, L. G. Presta, Y. G. Meng and D. C. Roopenian (2006). “Enhanced half-life of genetically engineered human IgG1 antibodies in a humanized FcRn mouse model: potential application in humorally mediated autoimmune disease.” Int Immunol 18 (12): 1759-1769.
Ridgway, J. B., L. G. Presta and P. Carter (1996). “Knobs-into-holes' engineering of antibody CH3 domains for heavy chain heterodimerization.” Protein Eng 9 (7): 617-621.
Scheraga, H. A. (2008). “From helix-coil transitions to protein folding.” Biopolymers 89 (5): 479-485.
Shields, R. L., A. K. Namenuk, K. Hong, Y. G. Meng, J. Rae, J. Briggs, D. Xie, J. Lai, A. Stadlen, B. Li, J. A. Fox and L. G. Presta (2001). “High resolution mapping of the binding site on human IgG1 for Fc gamma RI, Fc gamma RII, Fc gamma RIII, and FcRn and design of IgG1 variants with improved binding to the Fc gamma R.” J Biol Chem 276 (9): 6591-6604.
Shields, R. L., A. K. Namenuk, K. Hong, Y. G. Meng, J. Rae, J. Briggs, D. Xie, J. Lai, A. Stadlen, B. Li, J. A. Fox and L. G. Presta (2001). “High resolution mapping of the binding site on human IgG₁ for Fc gamma RI, Fc gamma RII, Fc gamma RIII, and FcRn and design of IgG₁ variants with improved binding to the Fc gamma R.” J Biol Chem 276 (9): 6591-6604.
Sun, Z. J., Y. Wu, W. H. Hou, Y. X. Wang, Q. Y. Yuan, H. J. Wang and M. Yu (2017). “A novel bispecific c-MET/PD-1 antibody with therapeutic potential in solid cancer.” Oncotarget 8 (17): 29067-29079.
Tam, S. H., S. G. McCarthy, A. A. Armstrong, S. Somani, S. J. Wu, X. Liu, A. Gervais, R. Ernst, D. Saro, R. Decker, J. Luo, G. L. Gilliland, M. L. Chiu and B. J. Scallon (2017). “Functional, Biophysical, and Structural Characterization of Human IgG1 and IgG4 Fc Variants with Ablated Immune Functionality.” Antibodies (Basel) 6 (3).
Troiani, T., E. Martinelli, M. Orditura, F. De Vita, F. Ciardiello and F. Morgillo (2012). “Beyond bevacizumab: new anti-VEGF strategies in colorectal cancer.” Expert Opin Investig Drugs 21 (7): 949-959.
Vafa, O., G. L. Gilliland, R. J. Brezski, B. Strake, T. Wilkinson, E. R. Lacy, B. Scallon, A. Teplyakov, T. J. Malia and W. R. Strohl (2014). “An engineered Fc variant of an IgG eliminates all immune effector functions via structural perturbations.” Methods 65 (1): 114-126.
Viticchie, G. and P. A. J. Muller (2015). “c-Met and Other Cell Surface Molecules: Interaction, Activation and Functional Consequences.” Biomedicines 3 (1): 46-70.
Wang, D. D., L. Ma, M. P. Wong, V. H. Lee and H. Yan (2015). “Contribution of EGFR and ErbB-3 Heterodimerization to the EGFR Mutation-Induced Gefitinib- and Erlotinib-Resistance in Non-Small-Cell Lung Carcinoma Treatments.” PLOS One 10 (5): e0128360.
Weidner, N., J. P. Semple, W. R. Welch and J. Folkman (1991). “Tumor angiogenesis and metastasis—correlation in invasive breast carcinoma.” N Engl J Med 324 (1): 1-8.
Worn, A. and A. Pluckthun (2001). “Stability engineering of antibody single-chain Fv fragments.” J Mol Biol 305 (5): 989-1010.
Wu, T. T. and E. A. Kabat (1970). “An analysis of the sequences of the variable regions of Bence Jones proteins and myeloma light chains and their implications for antibody complementarity.” J Exp Med 132 (2): 211-250.
Xu, D., M. L. Alegre, S. S. Varga, A. L. Rothermel, A. M. Collins, V. L. Pulito, L. S. Hanna, K. P. Dolan, P. W. Parren, J. A. Bluestone, L. K. Jolliffe and R. A. Zivin (2000). “In vitro characterization of five humanized OKT3 effector function variant antibodies.” Cell Immunol 200 (1): 16-26.
Zalevsky, J., A. K. Chamberlain, H. M. Horton, S. Karki, I. W. Leung, T. J. Sproule, G. A. Lazar, D. C. Roopenian and J. R. Desjarlais (2010). “Enhanced antibody half-life improves in vivo activity.” Nat Biotechnol 28 (2): 157-159.
Zhang, D., A. A. Armstrong, S. H. Tam, S. G. McCarthy, J. Luo, G. L. Gilliland and M. L. Chiu (2017). “Functional optimization of agonistic antibodies to OX40 receptor with novel Fc mutations to promote antibody multimerization.” MAbs 9:1129-1142.

1. An anti-EGFR antibody or an antigen-binding fragment thereof, comprising a heavy chain variable region comprising HCDR1, HCDR2, and HCDR3, wherein the HCDR1, HCDR2, and HCDR3 are selected from: SEQ ID NOs: 95, 96, and 97; SEQ ID NOs: 95, 96, and 98; SEQ ID NOs: 95, 96, and 105; SEQ ID NOs: 102, 100, and 101; and SEQ ID NOs: 102, 103, and 104; respectively.

2. The anti-EGFR antibody or antigen binding fragment of claim 1, comprising at least one antibody single domain having at least 85% identity to any one of SEQ ID NOs: 5-12 or antigen binding fragment thereof.

3. The anti-EGFR antibody or antigen binding fragment of claim 1, comprising tandem antibody single domain heavy chains having at least 85% identity to any one of SEQ ID NOs: 13-18 or antigen binding fragment thereof.

4. An anti-cMET antibody or an antigen-binding fragment thereof, comprising a heavy chain variable region comprising HCDR1, HCDR2, and HCDR3, and a light chain variable region comprising LCDR1, LCDR2, and LCDR3, wherein the HCDR1, HCDR2, and HCDR3 are selected from: SEQ ID NOs: 106, 107, and 133; SEQ ID NOs: 111, 112, and 113; SEQ ID NOs: 111, 114, and 113; SEQ ID NOs: 99, 118, and 119; SEQ ID NOs: 99, 120, and 119; SEQ ID NOs: 99, 121, and 119; SEQ ID NOs: 99, 122, and 119; respectively; and wherein the LCDR1, LCDR2, and LCDR3 are selected from: SEQ ID NOs: 108, 109, and 110; SEQ ID NOs: 115, 116, and 117; and SEQ ID NOs: 123, 124, and 125; respectively.

5. The anti-cMET antibody or antigen-binding fragment of claim 4, comprising a heavy chain sequence having at least 85% identity to any one of SEQ ID NOs: 24, 28-29, and 34-37, and a light chain sequence having at least 85% identity to any one of SEQ ID NOs: 26, 31-32, and 39-40.

6. The anti-cMET antibody or antigen-binding fragment of claim 4, comprising at least one cMET binding VHO sequence having at least 85% identity to any one of SEQ ID NOs: 41-44.

7. An anti-cMET antibody or an antigen-binding fragment thereof, comprising a heavy chain and a light chain selected from: heavy chain SEQ ID NO: 23 and light chain SEQ ID NO: 25; heavy chain SEQ ID NO: 23 and light chain SEQ ID NO: 26; heavy chain SEQ ID NO: 24 and light chain SEQ ID NO: 25; heavy chain SEQ ID NO: 24 and light chain SEQ ID NO: 26; heavy chain SEQ ID NO: 27 and light chain SEQ ID NO: 30; heavy chain SEQ ID NO: 27 and light chain SEQ ID NO: 31; heavy chain SEQ ID NO: 27 and light chain SEQ ID NO: 32; heavy chain SEQ ID NO: 28 and light chain SEQ ID NO: 30; heavy chain SEQ ID NO: 28 and light chain SEQ ID NO: 31; heavy chain SEQ ID NO: 28 and light chain SEQ ID NO: 32; heavy chain SEQ ID NO: 29 and light chain SEQ ID NO: 30; heavy chain SEQ ID NO: 29 and light chain SEQ ID NO: 31; heavy chain SEQ ID NO: 29 and light chain SEQ ID NO: 32; heavy chain SEQ ID NO: 33 and light chain SEQ ID NO: 38; heavy chain SEQ ID NO: 33 and light chain SEQ ID NO: 39; heavy chain SEQ ID NO: 33 and light chain SEQ ID NO: 40; heavy chain SEQ ID NO: 34 and light chain SEQ ID NO: 38; heavy chain SEQ ID NO: 34 and light chain SEQ ID NO: 39; heavy chain SEQ ID NO: 34 and light chain SEQ ID NO: 40; heavy chain SEQ ID NO: 35 and light chain SEQ ID NO: 38; heavy chain SEQ ID NO: 35 and light chain SEQ ID NO: 39; heavy chain SEQ ID NO: 35 and light chain SEQ ID NO: 40; heavy chain SEQ ID NO: 36 and light chain SEQ ID NO: 38; heavy chain SEQ ID NO: 36 and light chain SEQ ID NO: 39; heavy chain SEQ ID NO: 36 and light chain SEQ ID NO: 40; heavy chain SEQ ID NO: 37 and light chain SEQ ID NO: 38; heavy chain SEQ ID NO: 37 and light chain SEQ ID NO: 39; heavy chain SEQ ID NO: 37 and light chain SEQ ID NO: 40; respectively.

8. An anti-VEGF antibody or an antigen-binding fragment thereof, comprising a heavy chain variable region comprising HCDR1, HCDR2, and HCDR3, and a light chain variable region comprising LCDR1, LCDR2, and LCDR3, wherein the HCDR1, HCDR2, and HCDR3 are selected from: SEQ ID NOs: 129, 130, and 131; and SEQ ID NOs: 129, 132, and 131; respectively; and wherein the LCDR1, LCDR2, and LCDR3 are SEQ ID NOs: 126, 127, and 128, respectively.

9. The anti-VEGF antibody or antigen-binding fragment of claim 8, comprising an amino acid sequence having at least 85% identity to any one of SEQ ID NOs: 73-76.

10. A multispecific antibody comprising an EGFR arm that can bind EGFR and a cMet arm that can bind cMET,
wherein:
the EGFR arm comprises a heavy chain variable region comprising HCDR1, HCDR2, and HCDR3, wherein the HCDR1, HCDR2, and HCDR3 are selected from: SEQ ID NOs: 95, 96, and 97; SEQ ID NOs: 95, 96, and 98; SEQ ID NOs: 95, 96, and 105; SEQ ID NOs: 102, 100, and 101; and SEQ ID NOs: 102, 103, and 104; respectively, or
the EGFR arm comprises a light chain sequence and a heavy chain sequence selected from SEQ ID NOs: 1-4; a single domain heavy chain sequences selected from SEQ ID NOs: 5-12; or tandem single domain heavy chain sequences selected from SEQ ID NOs: 13-18; and
wherein:
the cMET arm comprises a heavy chain variable region comprising HCDR1, HCDR2, and HCDR3, and a light chain variable region comprising LCDR1, LCDR2, and LCDR3, wherein the HCDR1, HCDR2, and HCDR3 are selected from: SEQ ID NOs: 106, 107, and 133; SEQ ID NOs: 111, 112, and 113; SEQ ID NOs: 111, 114, and 113; SEQ ID NOs: 99, 118, and 119; SEQ ID NOs: 99, 120, and 119; SEQ ID NOs: 99, 121, and 119; SEQ ID NOs: 99, 122, and 119; respectively; and wherein the LCDR1, LCDR2, and LCDR3 are selected from: SEQ ID NOs: 108, 109, and 110; SEQ ID NOs: 115, 116, and 117; and SEQ ID NOs: 123, 124, and 125; respectively, or
the cMET arm comprises a light chain sequence and a heavy chain sequence selected from SEQ ID NO: 23-40 or at least one single domain heavy chain domain selected from SEQ ID NO: 41-44.

11. The multispecific antibody of claim 10, wherein the EGFR arm comprises at least one single domain heavy chain domain selected from SEQ ID NOs: 5-12.

12. The multispecific antibody of claim 10, wherein the EGFR arm comprises tandem single domain heavy chains selected from SEQ ID NOs: 13-18.

13. The multispecific antibody of claim 10, wherein the anti-EGFR arm comprises heavy chain SEQ ID NO: 1 and light chain SEQ ID NO: 2; heavy chain SEQ ID NO: 3 and light chain SEQ ID NO: 4; single domain heavy chain selected from SEQ ID NOs: 5-12; or tandem single domain heavy chain selected from SEQ ID NOs: 13-18.

14. The multispecific antibody of claim 10, wherein the cMET arm comprises a light chain sequence and a heavy chain sequence selected from SEQ ID NOs: 23-40.

15. The multispecific antibody of claim 10, wherein the cMET arm comprises at least one single domain heavy chain domain selected from SEQ ID NOs: 41-44.

16. The multispecific antibody of claim 10, wherein the anti-cMET arm comprises heavy chain SEQ ID NO: 23 and light chain SEQ ID NO: 25; heavy chain SEQ ID NO: 23 and light chain SEQ ID NO: 26; heavy chain SEQ ID NO: 24 and light chain SEQ ID NO: 25; heavy chain SEQ ID NO: 24 and light chain SEQ ID NO: 26; heavy chain SEQ ID NO: 27 and light chain SEQ ID NO: 30; heavy chain SEQ ID NO: 27 and light chain SEQ ID NO: 31; heavy chain SEQ ID NO: 27 and light chain SEQ ID NO: 32; heavy chain SEQ ID NO: 28 and light chain SEQ ID NO: 30; heavy chain SEQ ID NO: 28 and light chain SEQ ID NO: 31; heavy chain SEQ ID NO: 28 and light chain SEQ ID NO: 32; chain SEQ ID NO: 29 and light chain SEQ ID NO: 30; heavy chain SEQ ID NO: 29 and light chain SEQ ID NO: 31; heavy chain SEQ ID NO: 29 and light chain SEQ ID NO: 32; heavy chain SEQ ID NO: 33 and light chain SEQ ID NO: 38; heavy chain SEQ ID NO: 33 and light chain SEQ ID NO: 39; heavy chain SEQ ID NO: 33 and light chain SEQ ID NO: 40; heavy chain SEQ ID NO: 34 and light chain SEQ ID NO: 38; heavy chain SEQ ID NO: 34 and light chain SEQ ID NO: 39; heavy chain SEQ ID NO: 34 and light chain SEQ ID NO: 40; heavy chain SEQ ID NO: 35 and light chain SEQ ID NO: 38; heavy chain SEQ ID NO: 35 and light chain SEQ ID NO: 39; heavy chain SEQ ID NO: 35 and light chain SEQ ID NO: 40; heavy chain SEQ ID NO: 36 and light chain SEQ ID NO: 38; heavy chain SEQ ID NO: 36 and light chain SEQ ID NO: 39; heavy chain SEQ ID NO: 36 and light chain SEQ ID NO: 40; heavy chain SEQ ID NO: 37 and light chain SEQ ID NO: 39; or heavy chain SEQ ID NO: 37 and light chain SEQ ID NO: 40.

17. The multispecific antibody of claim 10, further comprising an anti-VEGF antibody or an antigen-binding fragment thereof comprising a heavy chain variable region comprising HCDR1, HCDR2, and HCDR3, and a light chain variable region comprising LCDR1, LCDR2, and LCDR3, wherein the HCDR1, HCDR2, and HCDR3 are selected from: SEQ ID NOs: 129, 130, and 131; and SEQ ID NOs: 129, 132, and 131; respectively; and wherein the LCDR1, LCDR2, and LCDR3 are SEQ ID NOs: 126, 127, and 128, respectively.

18. The multispecific antibody of claim 17, wherein the anti-VEGF antibody or antigen-binding fragment comprises an amino acid sequence having at least 85% identity to any one of SEQ ID NOs: 73-76.

19. The multispecific antibody of claim 10, further comprising an anti-PD-L1 antibody or an antigen-binding fragment thereof, comprising an amino acid sequence having at least 85% identity to any one of SEQ ID NOs: 71-72.

20. (canceled)

21. (canceled)

22. (canceled)

23. The multispecific antibody of claim 10, wherein the multispecific antibody comprises amino acid sequences selected from SEQ ID NOs: 83-94.

24. The multispecific antibody of claim 23, wherein the multispecific antibody comprises amino acid sequences set forth in SEQ ID NO: 90, SEQ ID NO: 84, and SEQ ID NO: 85, respectively.

25. A pharmaceutical composition comprising the antibody or antigen-binding fragment of claim 10.

26. A nucleic acid sequence encoding the antibody or antigen-binding fragment of claim 10.

27. A recombinant expression vector comprising the nucleic acid sequence of claim 26.

28. A recombinant expression transformant comprising the recombinant expression vector of claim 27.

29. A method for preparing an antibody or antigen-binding fragment, comprising: culturing the recombinant expression transformant of claim 28, and obtaining the antibody or antigen-binding fragment.

30. A method for treating or preventing a cancer in a subject, comprising administering to the subject a medicament comprising a pharmaceutically effective amount of the antibody or antigen-binding fragment of claim 10.

31. The method for treating or preventing a cancer of claim 30, wherein the cancer is lung cancer, breast cancer, pancreatic cancer, colorectal cancer, head and neck, esophageal, or gastric cancer.

32. The method for treating or preventing a cancer of claim 30, wherein the cancer is relapsed EGFR positive cancer.

33. A method for treating or preventing a disease in a subject, comprising administering to the subject a medicament comprising a pharmaceutically effective amount of the multispecific antibody of claim 10, wherein the disease is mediated by EGFR, VEGF or PD-L1, and cMET.


SEQUENCE LISTING (141 sequences)

SEQ ID NO: 1 - protein, 121 aa, synthetic construct [sequence omitted]

SEQ ID NO: 2 - protein, 106 aa, synthetic construct [sequence omitted]

SEQ ID NO: 3 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 4 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 5 - protein, 124 aa, synthetic construct
EVQLVESGGGVVRPGGSLRLSCAASGRTSRSYGMGWFRQAPGKEREFVSGISWRGDSTGYADSVKGRFTISRDNAKNSVDLQMNSLRAEDTALYYCAAAAGSAWYGTLYEYDYWGQGTQVTVSS

SEQ ID NO: 6 - protein, 124 aa, synthetic construct
EVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 7 - protein, 124 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGQGTQVTVSS

SEQ ID NO: 8 - protein, 124 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYDYDYWGQGTQVTVSS

SEQ ID NO: 9 - protein, 124 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGEGTQVTVSS

SEQ ID NO: 10 - protein, 124 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGEGTQVTVSS

SEQ ID NO: 11 - protein, 130 aa, synthetic construct
QVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSS

SEQ ID NO: 12 - protein, 127 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 13 - protein, 264 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGEGTQVTVSSGGGGSGGGGSQVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSS

SEQ ID NO: 14 - protein, 261 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGEGTQVTVSSGGGGSGGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 15 - protein, 264 aa, synthetic construct
QVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSGGGGSGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGEGTQVTVSS

SEQ ID NO: 16 - protein, 261 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGEGTQVTVSS

SEQ ID NO: 17 - protein, 269 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGEGTQVTVSSGGGGSGGGGSGGGGSQVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSS

SEQ ID NO: 18 - protein, 274 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGEGTQVTVSSGGGGSGGGGSGGGGSGGGGSQVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSS

SEQ ID NO: 19 - protein, 5 aa, synthetic construct
GGGGS

SEQ ID NO: 20 - protein, 10 aa, synthetic construct
GGGGSGGGGS

SEQ ID NO: 21 - protein, 15 aa, synthetic construct
GGGGSGGGGSGGGGS

SEQ ID NO: 22 - protein, 20 aa, synthetic construct
GGGGSGGGGSGGGGSGGGGS

SEQ ID NO: 23 - protein, 115 aa, synthetic construct [sequence omitted]

SEQ ID NO: 24 - protein, 115 aa, synthetic construct
QVQLVQSGAEVKKPGASVKVSCKASGYTFTDYYMHWVRQAPGQGLEWMGRVNPNRGGTTYAQKFQGRVTMTTDTSISTAYMELSRLRSDDTAVYYCARANWLDYWGQGTTVTVSS

SEQ ID NO: 25 - protein, 108 aa, synthetic construct [sequence omitted]

SEQ ID NO: 26 - protein, 108 aa, synthetic construct
DIQMTQSPSSLSASVGDRVTITCRVSQSVSSIYLHWYQQKPGKAPKLLIYSTSNLQSGVPSRFSGSGSGTDFTLTISSLQPEDFATYYCQVYSGYPLTFGGGTKVEIK

SEQ ID NO: 27 - protein, 118 aa, synthetic construct
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 28 - protein, 118 aa, synthetic construct
QVQLVQSGAEVKKPGSSVKVSCKASGYIFTAYTMHWVRQAPGQGLEWMGGIKPNNGLANYAQKFQGRVTITADESTSTAYMELSSLRSEDTAVYYCARSEITTEFDYWGQGTLVTVSS

SEQ ID NO: 29 - protein, 118 aa, synthetic construct
QVQLQESGPGLVKPSGTLSLTCAVSGYIFTAYTMHWVRQPPGKGLEWIGGIKPNNGLANYNPSLKSRATLSVDKSKNQASMKLSSVTAADTAVYYCARSEITTEFDYWGQGTLVTVSS

SEQ ID NO: 30 - protein, 111 aa, synthetic construct
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 31 - protein, 111 aa, synthetic construct
DIVMTQTPLSSPVTLGQPASISCRASESVDSYANSFMHWLQQRPGQPPRLLIYRASNLESGVPDRFSGSGAGTDFTLKISRVEAEDVGVYYCQQSKEDPLTFGGGTKVEIK

SEQ ID NO: 32 - protein, 111 aa, synthetic construct
DIQMTQSPSSLSASVGDRVTITCRASESVDSYANSFMHWYQQKPGKAPKLLIYRASNLESGVPSRFSGSGSGTDFTLTISSLQPEDFATYYCQQSKEDPLTFGGGTKVEIK

SEQ ID NO: 33 - protein, 119 aa, synthetic construct [sequence omitted]

SEQ ID NO: 34 - protein, 119 aa, synthetic construct
QVQLVQSGAEVKKPGSSVKMSCKASGYTFTSYWLHWVRQAPGQGLEWIGMIDPSNSDTRFAQKFQGRATLTADESTSTAYMELSSLRSEDTAVYYCATYGSYVSPLDYWGQGTTVTVSS

SEQ ID NO: 35 - protein, 119 aa, synthetic construct
QVQLQESGPGLVKPSDTLSLTCAASGYTFTSYWLHWVRQPPGKGLEWIGMIDPSNSDTRFNPSLKSRVTMSVDTSKNQASLKLSSVTAVDTAVYYCATYGSYVSPLDYWGQGTTVTVSS

SEQ ID NO: 36 - protein, 119 aa, synthetic construct
EVQLVESGGGLVQPGGSLRLSCAASGYTFTSYWLHWVRQAPGKGLEWVGMIDPSNSDTRYAASVKGRFTISVDDSKNSLYLQMNSLKTEDTAVYYCATYGSYVSPLDYWGQGTTVTVSS

SEQ ID NO: 37 - protein, 119 aa, synthetic construct
EVQLVESGGGLVKPGGSVRMSCAASGYTFTSYWLHWVRQAPGKGLEWIGMIDPSNSDTRYAAPFKGRATLSVDDSKNTAYMQLNSLKTEDTAVYYCATYGSYVSPLDYWGQGTTVTVSS

SEQ ID NO: 38 - protein, 113 aa, synthetic construct [sequence omitted]

SEQ ID NO: 39 - protein, 113 aa, synthetic construct
DIQMTQSPSTLSASVGDRVTITCRSSQSLLYTSSQKNYLAWYQQKPGKAPKLLIYWASTRESGVPSRFSGSGSGTEFTLTISSVQPDDFATYYCQQYYAYPWTFGQGTKLEIK

SEQ ID NO: 40 - protein, 113 aa, synthetic construct
DIVMTQTPPSLPVNPGEPASVSCRSSQSLLYTSSQKNYLAWYLQKPGQSPQLLIYWASTRESGVPDRFSGSGSGSDFTLKISWVEAEDLGVYYCQQYYAYPWTFGQGTKLEIK

SEQ ID NO: 41 - protein, 125 aa, synthetic construct
DVQLVESGGGLVQPGGSLRLSCAASGFILDYYAIGWFRQAPGKEREGVLCIDASDDITYYADSVKGRFTISRDNSKNTVYLQMNSLRPEDTAVYYCATPIGLSSSCLLEYDYDYWGQGTLVTVSS

SEQ ID NO: 42 - protein, 125 aa, synthetic construct
EVQLVESGGGLVQPGGSLRLSCAASGFILDYYAIGWFRQAPGKEREGVLCIDASDDITYYADSVKGRFTISRDNSKNTVYLQMNSLRAEDTAVYYCATPIGLSSSCLLEYDYDYWGQGTLVTVSS

SEQ ID NO: 43 - protein, 124 aa, synthetic construct
EVQLVESGGGLVQAGGSLRLSCAASGFTFDDYAIGWFRQAPGEEREGVSSISSTYGLTYYADSVKGRFTISSSNAKNTVYLQMNNLKPEDTAVYYCAATPIERLGLDAYEYDYWGQGTQVTVSS

SEQ ID NO: 44 - protein, 124 aa, synthetic construct
EVQLVESGGGLVQPGGSLRLSCAASGFTFDDYAIGWFRQAPGEEREGVSSISSTYGLTYYADSVKGRFTISSDNSKNTVYLQMNSLRAEDTAVYYCAATPIERLGLDAYEYDYWGQGTQVTVSS

SEQ ID NO: 45 - protein, 95 aa, synthetic construct
EIDQCIVDDITYNVQDTFHKKHEEGHMLNCTCFGQGRGRWKCSPVDQCQDSETGTFYQIGDSWEKYVHGVRYQCYCYGRGIGEWHCQPLQTYPSS

SEQ ID NO: 46 - protein, 95 aa, synthetic construct
EIDQCIVDDITYNVQDTFHKKHEEGHMLNCTCFGQGRGRWKCEPVDQCQDSETGTFYQIGDSWEKYVHGVRYQCYCYGRGIGEWHCQPLQTYPSS

SEQ ID NO: 47 - protein, 95 aa, synthetic construct
EIDQCIVDDITYNVQDTFHKKHEEGHMLQCTCFGQGRGRWKCDPVDQCQDSETGTFYQIGDSWEKYVHGVRYQCYCYGRGIGEWHCQPLQTYPSS

SEQ ID NO: 48 - protein, 25 aa, synthetic construct
EVGQRGVVGLPGQRGERGFPGLPGY

SEQ ID NO: 49 - protein, 15 aa, synthetic construct
EPKSCDKTHTCPPCP

SEQ ID NO: 50 - protein, 21 aa, synthetic construct
GIVEECCFRSCDLALLETYCA

SEQ ID NO: 51 - protein, 28 aa, synthetic construct
AYRPSETLCGGELVDTLQFVCGDRGFYF

SEQ ID NO: 52 - protein, 129 aa, synthetic construct
QVQLQESGGGLVQAGGSLRLSCAVSGNTISRYATGWFRQTPGNEREFVAAIRWTNGNTYYADSVEGRFTISRDSGKNTVYLQMNNLQPEDTAVYYCASRFLPYASSNAYHEALYNYDYWGQGTQVTVSS

SEQ ID NO: 53 - protein, 129 aa, synthetic construct
QVQLQESGGGLVQAGGSLRLSCAVSGNTISRYATGWFRQTPGNEREFVAAIRWTNGNTYYADSVEGRFTISRDSGKNTVYLQMNNLQPEDTAVYYCASRFLPYASSNAYHESLYNYDYWGQGTQVTVSS

SEQ ID NO: 54 - protein, 129 aa, synthetic construct
QVQLQESGGGLVQAGGSLRLSCAVSGNTISRYATGWFRQTPGNEREFVAAIRWENGNTYYADSVEGRFTISRDSGKNTVYLQMNNLQPEDTAVYYCASRFLPYASSNAYHEALYNYDYWGQGTQVTVSS

SEQ ID NO: 55 - protein, 129 aa, synthetic construct
QVQLQESGGGLVQAGGSLRLSCAVSGNTISRYATGWFRQTPGNEREFVAAIRWENGNTYYADSVEGRFTISRDSGKNTVYLQMNNLQPEDTAVYYCASRFLPYASSNAYHESLYNYDYWGQGTQVTVSS

SEQ ID NO: 56 - protein, 113 aa, synthetic construct
QVQLQESGGGLVQAGGSLRLSCAVSGNTISRYATGWFRQTPGNEREFVAAIRWTNGNTYYADSVEGRFTISRDSGKNTVYLQRFLPYASSNAYHEALYNYDYWGQGTQVTVSS

SEQ ID NO: 57 - protein, 113 aa, synthetic construct
QVQLQESGGGLVQAGGSLRLSCAVSGNTISRYATGWFRQTPGNEREFVAAIRWENGNTYYADSVEGRFTISRDSGKNTVYLQRFLPYASSNAYHESLYNYDYWGQGTQVTVSS

SEQ ID NO: 58 - protein, 129 aa, synthetic construct
QVQLQESGGGLVQAGGSLRLSCAVSGNTLSRYAMGWFRQAPGNEREFVAAIRWNNGNTHYADSVKGRFTISRDSAKNTVYLQMNNLQPEDTAVYYCASRFLPYASSNAYHEALYNYDYWGQGTQVTVSS

SEQ ID NO: 59 - protein, 129 aa, synthetic construct
EVQLVESGGGLVQPGGSLRLSCAASGNTLSRYAMGWFRQAPGKEREFVAAIRWNNGNTHYVDSVKGRFTISRDNAKNSVYLQMNSLRAEDTAVYYCASRFLPYASSNAYHEALYNYDYWGQGTLVTVSS

SEQ ID NO: 60 - protein, 129 aa, synthetic construct
QVTLKESGPVLVKPTETLTLTCTVSGNTLSRYAMGWFRQAPGKEREFVAAIRWNNGNTHYSTSLKSRLTISKDTSKSQVVLTMTNMDPVDTATYYCASRFLPYASSNAYHEALYNYDYWGQGTLVTVSS

SEQ ID NO: 61 - protein, 129 aa, synthetic construct
QVQLVQSGAEVKKPGASVKVSCKASGNTLSRYAMGWFRQAPGQEREFVAAIRWNNGNTHYAQKFQGRVTMTRDTSTSTVYMELSSLRSEDTAVYYCASRFLPYASSNAYHEALYNYDYWGQGTLVTVSS

SEQ ID NO: 62 - protein, 6 aa, synthetic construct
GPLGVR

SEQ ID NO: 63 - protein, 6 aa, synthetic construct
PLGLAR

SEQ ID NO: 64 - protein, 6 aa, synthetic construct
PLGLAG

SEQ ID NO: 65 - protein, 8 aa, synthetic construct
IPVSLRSG

SEQ ID NO: 66 - protein, 8 aa, synthetic construct
GPLGMLSQ

SEQ ID NO: 67 - protein, 10 aa, synthetic construct
RPKPVEVWRK

SEQ ID NO: 68 - protein, 8 aa, synthetic construct
LSGRSDNH

SEQ ID NO: 69 - protein, 8 aa, synthetic construct
TGRGPSWV

SEQ ID NO: 70 - protein, 271 aa, Homo sapiens
FTVTVPKDLYVVYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 71 - protein, 240 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 72 - protein, 235 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSS

SEQ ID NO: 73 - protein, 245 aa, synthetic construct
DIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKGGGGSGGGGSGGGGSQVQLVQSGSELKKPGASVKVSCKASGYTFTNYGMNWVRQAPGQGLEWMGWINTYTGEPTYAQGFTGRFVFSLDTSVSTAYLQICSLKAEDTAVYFCAKYPHYYGSSHWYFDVWGQGTLVTVSS

SEQ ID NO: 74 - protein, 245 aa, synthetic construct
DIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKGGGGSGGGGSGGGGSQVQLVQSGAEVKKPGSSVKVSCKASGYTFTNYGMNWVRQAPGQGLEWMGWINTYTGEPTYAQKFQGRFTFTADESTSTAYMELSSLRSEDTAVYYCAKYPHYYGSSHWYFDVWGQGTLVTVSS

SEQ ID NO: 75 - protein, 245 aa, synthetic construct
QVQLVQSGSELKKPGASVKVSCKASGYTFTNYGMNWVRQAPGQGLEWMGWINTYTGEPTYAQGFTGRFVFSLDTSVSTAYLQICSLKAEDTAVYFCAKYPHYYGSSHWYFDVWGQGTLVTVSSGGGGSGGGGSGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIK

SEQ ID NO: 76 - protein, 245 aa, synthetic construct
QVQLVQSGAEVKKPGSSVKVSCKASGYTFTNYGMNWVRQAPGQGLEWMGWINTYTGEPTYAQKFQGRFTFTADESTSTAYMELSSLRSEDTAVYYCAKYPHYYGSSHWYFDVWGQGTLVTVSSGGGGSGGGGSGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIK

SEQ ID NO: 77 - protein, 119 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 78 - protein, 107 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 79 - protein, 125 aa, synthetic construct [sequence omitted]

SEQ ID NO: 80 - protein, 107 aa, synthetic construct [sequence omitted]

SEQ ID NO: 81 - protein, 108 aa, synthetic construct
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERK

SEQ ID NO: 82 - protein, 127 aa, synthetic construct
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSS

SEQ ID NO: 83 - protein, 496 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGEGTQVTVSSGGGGSGGGGSQVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSGGGGSDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPCREEMTKNQVSLWCLVKGFYPSDIAVEWESNGQPENNYKTTPLVLDSDGSFFLYSKLTVDKSRWQQGNVFSCSVMHEALHNHYTQKSLSLSPGK

SEQ ID NO: 84 - protein, 445 aa, synthetic construct
QVQLVQSGAEVKKPGASVKVSCKASGYTFTDYYMHWVRQAPGQGLEWMGRVNPNRGGTTYAQKFQGRVTMTTDTSISTAYMELSRLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVCTLPPSREEMTKNQVSLSCAVKGFYPSDIAVEWESNGQPENNYKTTPLVLDSDGSFFLVSKLTVDKSRWQQGNVFSCSVMHEALHNHYTQKSLSLSPGK

SEQ ID NO: 85 - protein, 215 aa, synthetic construct
DIQMTQSPSSLSASVGDRVTITCRVSQSVSSIYLHWYQQKPGKAPKLLIYSTSNLQSGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 763 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGEGTQVTVSSGGGGSGGGGSQVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSGGGGSDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPCREEMTKNQVSLWCLVKGFYPSDIAVEWESNGQPENNYKTTPLVLDSDGSFFLYSKLTVDKSRWQQGNVFSCSVMHEALHNHYTQKSLSLSPGKGGGGSPLGLAGGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKGGSEGKSSGSGSESKSTGGSEQVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSS

SEQ ID NO: 87 - protein, 712 aa, synthetic construct
QVQLVQSGAEVKKPGASVKVSCKASGYTFTDYYMHWVRQAPGQGLEWMGRVNPNRGGTTYAQKFQGRVTMTTDTSISTAYMELSRLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVCTLPPSREEMTKNQVSLSCAVKGFYPSDIAVEWESNGQPENNYKTTPLVLDSDGSFFLVSKLTVDKSRWQQGNVFSCSVMHEALHNHYTQKSLSLSPGKGGGGSPLGLAGGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKGGSEGKSSGSGSESKSTGGSEQVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSS

SEQ ID NO: 88 - protein, 763 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGEGTQVTVSSGGGGSGGGGSQVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSGGGGSDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPCREEMTKNQVSLWCLVKGFYPSDIAVEWESNGQPENNYKTTPLVLDSDGSFFLYSKLTVDKSRWQQGNVFSCSVMHEALHNHYTQKSLSLSPGKGGGGSPLGLAGGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGCGTKLEIKGGSEGKSSGSGSESKSTGGSEQVQLVQSGAEVKKPGSSVKVSCKASGYTFTNYGMNWVRQAPGQCLEWMGWINTYTGEPTYAQKFQGRVTITADESTSTAYMELSSLRSEDTAVYYCAKYPHYYGSSHWYFDVWGQGTLVTVSS

SEQ ID NO: 89 - protein, 712 aa, synthetic construct
QVQLVQSGAEVKKPGASVKVSCKASGYTFTDYYMHWVRQAPGQGLEWMGRVNPNRGGTTYAQKFQGRVTMTTDTSISTAYMELSRLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVCTLPPSREEMTKNQVSLSCAVKGFYPSDIAVEWESNGQPENNYKTTPLVLDSDGSFFLVSKLTVDKSRWQQGNVFSCSVMHEALHNHYTQKSLSLSPGKGGGGSPLGLAGGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGCGTKLEIKGGSEGKSSGSGSESKSTGGSEQVQLVQSGAEVKKPGSSVKVSCKASGYTFTNYGMNWVRQAPGQCLEWMGWINTYTGEPTYAQKFQGRVTITADESTSTAYMELSSLRSEDTAVYYCAKYPHYYGSSHWYFDVWGQGTLVTVSS

SEQ ID NO: 90 - protein, 767 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGEGTQVTVSSGGGGSGGGGSQVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSGGGGSDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPCREEMTKNQVSLWCLVKGFYPSDIAVEWESNGQPENNYKTTPLVLDSDGSFFLYSKLTVDKSRWQQGNVFSCSVMHEALHNHYTQKSLSLSPGKGGGGSGGGGSGGGGSGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKGGSEGKSSGSGSESKSTGGSEQVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSS

SEQ ID NO: 91 - protein, 716 aa, synthetic construct
QVQLVQSGAEVKKPGASVKVSCKASGYTFTDYYMHWVRQAPGQGLEWMGRVNPNRGGTTYAQKFQGRVTMTTDTSISTAYMELSRLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVCTLPPSREEMTKNQVSLSCAVKGFYPSDIAVEWESNGQPENNYKTTPLVLDSDGSFFLVSKLTVDKSRWQQGNVFSCSVMHEALHNHYTQKSLSLSPGKGGGGSGGGGSGGGGSGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGQGTKLEIKGGSEGKSSGSGSESKSTGGSEQVQLVQSGAEVKKPGSSVKVSCKASGYTFTNYGMNWVRQAPGQGLEWMGWINTYTGEPTYAQKFQGRVTITADESTSTAYMELSSLRSEDTAVYYCAKYPHYYGSSHWYFDVWGQGTLVTVSS

SEQ ID NO: 92 - protein, 768 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGWAWYGTLYEYDYWGEGTQVTVSSGGGGSGGGGSQVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSGGGGSDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPCREEMTKNQVSLWCLVKGFYPSDIAVEWESNGQPENNYKTTPLVLDSDGSFFLYSKLTVDKSRWQQGNVFSCSVMHEALHNHYTQKSLSLSPGKGGGGSGGGGGSGGGGSGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGCGTKLEIKGGSEGKSSGSGSESKSTGGSEQVQLVQSGAEVKKPGSSVKVSCKASGYTFTNYGMNWVRQAPGQCLEWMGWINTYTGEPTYAQKFQGRVTITADESTSTAYMELSSLRSEDTAVYYCAKYPHYYGSSHWYFDVWGQGTLVTVSS

SEQ ID NO: 93 - protein, 716 aa, synthetic construct
QVQLVQSGAEVKKPGASVKVSCKASGYTFTDYYMHWVRQAPGQGLEWMGRVNPNRGGTTYAQKFQGRVTMTTDTSISTAYMELSRLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVCTLPPSREEMTKNQVSLSCAVKGFYPSDIAVEWESNGQPENNYKTTPLVLDSDGSFFLVSKLTVDKSRWQQGNVFSCSVMHEALHNHYTQKSLSLSPGKGGGGSGGGGSGGGGSGGGGSDIQMTQSPSSLSASVGDRVTITCRASQDISNYLNWYQQKPGKAPKVLIYFTSSLHSGVPSRFSGSGSGTDFTLTISSLQPEDFATYYCQQYSTVPWTFGCGTKLEIKGGSEGKSSGSGSESKSTGGSEQVQLVQSGAEVKKPGSSVKVSCKASGYTFTNYGMNWVRQAPGQCLEWMGWINTYTGEPTYAQKFQGRVTITADESTSTAYMELSSLRSEDTAVYYCAKYPHYYGSSHWYFDVWGQGTLVTVSS

SEQ ID NO: 94 - protein, 710 aa, synthetic construct
QVQLVQSGAEVKKPGASVKVSCKASGYTFTDYYMHWVRQAPGQGLEWMGRVNPNRGGTTYAQKFQGRVTMTTDTSISTAYMELSRLRSDDTAVYYCARANWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVCTLPPSREEMTKNQVSLSCAVKGFYPSDIAVEWESNGQPENNYKTTPLVLDSDGSFFLVSKLTVDKSRWQQGNVFSCSVMHEALHNHYTQKSLSLSPGKGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGCGTKVEIKGGSEGKSSGSGSESKSTGGSEVQLVESGGGLVQPGGSLRLSCAASGFTFSDSWIHWVRQAPGKCLEWVAWISPYGGSTYYADSVKGRFTISADTSKNTAYLQMNSLRAEDTAVYYCARRHWPGGFDYWGQGTLVTVSS

SEQ ID NO: 95 - protein, 5 aa, synthetic construct
SYGMG

SEQ ID NO: 96 - protein, 17 aa, synthetic construct
GISWRGDSTGYADSVKG

SEQ ID NO: 97 - protein, 15 aa, synthetic construct
AAGSAWYGTLYEYDY

SEQ ID NO: 98 - protein, 15 aa, synthetic construct
AAGWAWYGTLYEYDY

SEQ ID NO: 99 - protein, 5 aa, synthetic construct
SYWLH

SEQ ID NO: 100 - protein, 16 aa, synthetic construct
IRWSGGYTYYTDSVKG

SEQ ID NO: 101 - protein, 21 aa, synthetic construct
TYLSSDYSRYALPQRPLDYDY

SEQ ID NO: 102 - protein, 5 aa, synthetic construct
SYAMG

SEQ ID NO: 103 - protein, 19 aa, synthetic construct
AINWSSGSTYYADSVKGRF

SEQ ID NO: 104 - protein, 18 aa, synthetic construct
GYQINSGNYNFKDYEYDY

SEQ ID NO: 105 - protein, 15 aa, synthetic construct
AAGSAWYGTLYDYDY

SEQ ID NO: 106 - protein, 5 aa, synthetic construct
DYYMH

SEQ ID NO: 107 - protein, 17 aa, synthetic construct
RVNPNRGGTTYAQKFQG

SEQ ID NO: 108 - protein, 12 aa, synthetic construct
RVSQSVSSIYLH

SEQ ID NO: 109 - protein, 7 aa, synthetic construct
STSNLQS

SEQ ID NO: 110 - protein, 9 aa, synthetic construct
QVYSGYPLT

SEQ ID NO: 111 - protein, 5 aa, synthetic construct
AYTMH

SEQ ID NO: 112 - protein, 17 aa, synthetic construct
GIKPNNGLANYAQKFQG

SEQ ID NO: 113 - protein, 9 aa, synthetic construct
SEITTEFDY

SEQ ID NO: 114 - protein, 17 aa, synthetic construct
GIKPNNGLANYNPSLKS

SEQ ID NO: 115 - protein, 15 aa, synthetic construct
RASESVDSYANSFMH

SEQ ID NO: 116 - protein, 7 aa, synthetic construct
RASNLES

SEQ ID NO: 117 - protein, 9 aa, synthetic construct
QQSKEDPLT

SEQ ID NO: 118 - protein, 17 aa, synthetic construct
MIDPSNSDTRFAQKFQG

SEQ ID NO: 119 - protein, 10 aa, synthetic construct
YGSYVSPLDY

SEQ ID NO: 120 - protein, 17 aa, synthetic construct
MIDPSNSDTRFNPSLKS

SEQ ID NO: 121 - protein, 17 aa, synthetic construct
MIDPSNSDTRYAASVKG

SEQ ID NO: 122 - protein, 17 aa, synthetic construct
MIDPSNSDTRYAAPFKG

SEQ ID NO: 123 - protein, 17 aa, synthetic construct
RSSQSLLYTSSQKNYLA

SEQ ID NO: 124 - protein, 7 aa, synthetic construct
WASTRES

SEQ ID NO: 125 - protein, 9 aa, synthetic construct
QQYYAYPWT

SEQ ID NO: 126 - protein, 11 aa, synthetic construct
RASQDISNYLN

SEQ ID NO: 127 - protein, 7 aa, synthetic construct
FTSSLHS

SEQ ID NO: 128 - protein, 9 aa, synthetic construct
QQYSTVPWT

SEQ ID NO: 129 - protein, 5 aa, synthetic construct
NYGMN

SEQ ID NO: 130 - protein, 17 aa, synthetic construct
WINTYTGEPTYAQGFTG

SEQ ID NO: 131 - protein, 14 aa, synthetic construct
YPHYYGSSHWYFDV

SEQ ID NO: 132 - protein, 17 aa, synthetic construct
WINTYTGEPTYAQKFQG

SEQ ID NO: 133 - protein, 6 aa, synthetic construct
ANWLDY

SEQ ID NO: 134 - protein, 5 aa, synthetic construct
RYATG

SEQ ID NO: 135 - protein, 17 aa, synthetic construct
AIRWTNGNTYYADSVEG

SEQ ID NO: 136 - protein, 20 aa, synthetic construct
RFLPYASSNAYHEALYNYDY

SEQ ID NO: 137 - protein, 20 aa, synthetic construct
RFLPYASSNAYHESLYNYDY

SEQ ID NO: 138 - protein, 17 aa, synthetic construct
AIRWENGNTYYADSVEG

SEQ ID NO: 139 - protein, 10 aa, synthetic construct
GNTISRYATG

SEQ ID NO: 140 - protein, 10 aa, synthetic construct
GNTLSRYAMG

SEQ ID NO: 141 - protein, 17 aa, synthetic construct
AIRWNNGNTHYADSVKG